(19) United States
(12) Patent Application Publication
(10) Pub. No.: US 2012/0269814 A1
(43) Pub. Date: Oct. 25, 2012

(54) ANTI-C MPL ANTIBODIES

(75) Inventors: Ping Wei, Agoura Hills, CA (US); Aaron George Winters, Ventura, CA (US); Christina Abbott, Newbury Park, CA (US)

(73) Assignee: AMGEN INC., Thousand Oaks, CA (US)

(21) Appl. No.: 13/508,973

(22) PCT Filed: Nov. 10, 2010
(86) PCT No.: PCT/US10/56245

§ 371 Date: Jun. 27, 2012

Related U.S. Application Data
(60) Provisional application No. 61/259,966, filed on Nov. 10, 2009.
(60) Provisional application No. 61/373,755, filed on Aug. 13, 2010.

(51) Int. Cl. C07K 16/30; C12N 15/13; G01N 33/577; C12N 5/16; C12P 21/02; G01N 33/574; A61K 39/395; C12N 15/85

(52) U.S. Cl. 424/138.1; 530/387.7; 530/387.3; 536/23.1; 435/320.1; 435/330; 435/70.2; 435/7.23

(57) ABSTRACT

The present invention provides compositions and methods utilizing antagonistic antibodies which specifically bind to human c-MPL (e.g., TPO-R).

### RELATED APPLICATIONS

This application is the National Stage of International Application No. PCT/US2010/056245, filed Nov. 10, 2010, which claims the benefit of U.S. Provisional Application No. 61/259,966, filed on Nov. 10, 2009, and of U.S. Provisional Application No. 61/373,755, filed on Aug. 13, 2010, the contents of which are hereby incorporated by reference in their entirety.

### FIELD OF THE INVENTION

The field of the invention relates to compositions and methods utilizing anti-c-MPL antagonistic antibodies.

### BACKGROUND OF THE INVENTION

TPO and its cognate receptor, c-Mpl (myeloproliferative leukemia virus oncogene), are primary physiological regulators of megakaryopoiesis and play important roles in platelet production and hemostasis (Battinelli et al., Curr Opin Hematol (2007)14:419-426; Deutsch et al., Br J Haematol (2006)134:453-466; Kaushansky et al., J Clin Invest (2005)115:3339-3347). Human c-Mpl is a 635 amino acid transmembrane protein and is a member of the single-chain, type I cytokine receptor subfamily of hematopoietic growth factor receptors that also includes the receptors for erythropoietin (Epo), granulocyte colony-stimulating factor (G-CSF), growth hormone (GH), and prolactin (PL) (Boulay et al., Immunity (2003)19:159-163). These receptors do not possess an intracellular kinase domain and require ligand-induced homodimerization followed by the association and activation of Janus kinases (JAKs) for signal transduction. Binding of TPO to c-Mpl leads to phosphorylation of receptor-bound JAK2 and the subsequent activation of several downstream signaling pathways, including the signal transducers and activators of transcription (STAT) pathway, the mitogen-activated protein kinase (MAPK) pathways involving both the extracellular signal-regulated kinases (ERK) and p38 kinases, and the phosphatidylinositol-3-kinase (PI3K) pathway (Kaushansky et al., J Clin Invest (2005)115:3339-3347).

Numerous agonistic antibodies against the c-MPL receptor have been described, such as for example, in U.S. Pat. No. 6,342,220; U.S. Pat. No. 6,737,249; WO 2005/056604; WO 2009/041734; WO 2007/108559; Kai et al., Bio Industry (2008, 25(7), 83-91; Tuschiya, M, Saibo Kogaku (2007), 26(3), 268-273; WO 2005/107784; WO 2005/056602; WO 2002/033072; WO 99/03495; Deng et al., Blood (1998), 92(6), 1981-1988; among others.

In addition, a mAb against human c-Mpl, designated M1, was developed as a reagent and used in various immunoassays (Debili et al., Blood (1995)85:391-401). Many other anti-c-Mpl Abs have been generated, but the details of their generation and characterization have been sparse except for one: BAH-1 (Deng et al., Blood (1998)92:1981-1988). However for BAH-1, the reported validation studies were incomplete or not reproducible.

Although anti-c-MPL antibodies have been described, there remains a need for highly specific anti-c-MPL antibodies that antagonize or neutralize c-MPL activity. In addition, there remains a need for a specific and thoroughly validated antibody against human c-Mpl to facilitate accurate studies of the expression and regulation of this receptor.

### SUMMARY OF THE INVENTION

Provided herein are novel anti-c-MPL antibodies that have increased specificity for human c-MPL; and are able to detect c-MPL. In another aspect, the invention anti-c-MPL antibodies have antagonistic/neutralizing activity such that they are useful inhibiting/antagonizing TPO/c-MPL biological activity Inhibition of TPO/-c-MPL bioactivity is useful for treating certain diseases such as cancer (e.g., leukemias) and the like.

In particular, the following items of the invention are provided:

1. An isolated antagonistic anti-c-MPL antibody, wherein said antibody competes for binding with at least one antibody selected from the group consisting of mAb-1.75, mAb-1.6, and mAb-1.111.

2. The antibody of claim 1, wherein said antibody competes for binding with at least two antibodies selected from the group consisting of mAb-1.75, mAb-1.6, and mAb-1.111.

3. The antibody of claim 2, wherein said antibody competes for binding with each of mAb-1.75, mAb-1.6, and mAb-1.111.

4. The antibody of claims 1-3, wherein said antibody binds to the epitope corresponding to -PWQDGPK- (SEQ ID NO: 97).

5. An isolated antagonistic anti-c-MPL antibody, wherein said antibody competes for binding with at least one antibody selected from the group consisting of mAb-1.78, mAb-1.36, and mAb-1.169.

6. The antibody of claim 5, wherein said antibody competes for binding with at least two antibodies selected from the group consisting of mAb-1.78, mAb-1.36, and mAb-1.169.

7. The antibody of claim 6, wherein said antibody competes for binding with each of mAb-1.78, mAb-1.36, and mAb-1.169.

8. An isolated antagonistic anti-c-MPL antibody, wherein said antibody has a K_{D} of greater than at least 700 pM.

9. The antibody of claim 8, wherein said antibody has a K_{D} of greater than at least 500 pM.

10. The antibody of claim 9, wherein said antibody has a K_{D} of greater than at least 100 pM.

11. A purified antibody or fragment of an antibody, wherein the antibody or the fragment specifically binds to c-MPL and has at least two or more of the characteristics selected from the group consisting of:
(a) affinity (kD) greater than at least 500 pM to c-MPL;
(b) affinity (kD) greater than at least 100 pM to c-MPL;
(c) reduces the growth of human bone marrow (CD34+ DC38−) progenitor cells in a mammal by at least 50%; and
(d) binds the extracellular domain of c-MPL.

12. A purified/isolated antibody or fragment of an antibody, wherein the antibody or the fragment specifically binds to human c-MPL and comprises (A-1/mAb1.6):
(a) a VH CDR1 having an amino acid sequence identical to or comprising 1, 2, or 3 amino acid residue substitutions relative to SEQ ID NO: 20;
(b) a VH CDR2 having an amino acid sequence identical to or comprising 1, 2, or 3 amino acid residue substitutions relative to SEQ ID NO: 22;
(c) a VH CDR3 having an amino acid sequence identical to or comprising 1, 2, or 3 amino acid residue substitutions relative to SEQ ID NO: 24;
(d) a VL CDR1 having an amino acid sequence identical to or comprising 1, 2, or 3 amino acid residue substitutions relative to SEQ ID NO: 2;
(e) a VL CDR2 having an amino acid sequence identical to or comprising 1, 2, or 3 amino acid residue substitutions relative to SEQ ID NO: 4; and
(f) a VL CDR3 having an amino acid sequence identical to or comprising 1, 2, or 3 amino acid residue substitutions relative to SEQ ID NO: 6.

13. A purified/isolated antibody or fragment of an antibody, wherein the antibody or the fragment specifically binds to human c-MPL and comprises:
(a) a VH CDR1 having the amino acid sequence of SEQ ID NO: 20;
(b) a VH CDR2 having the amino acid sequence of SEQ ID NO: 22
(c) a VH CDR3 having the amino acid sequence of SEQ ID NO: 24;
(d) a VL CDR1 having the amino acid sequence of SEQ ID NO: 2;
(e) a VL CDR2 having the amino acid sequence of SEQ ID NO: 4; and
(f) a VL CDR3 having the amino acid sequence of SEQ ID NO: 6.

14. A purified antibody or antibody fragment, wherein the antibody or the fragment (i) comprises a VH chain domain comprising three CDRs and a VL chain domain comprising three CDRs; and (ii) specifically binds human c-MPL, wherein the three CDRs of the VH chain domain comprise:
(a) a VH CDR1 comprising the amino acid sequence of SEQ ID NO: 20;
(b) a VH CDR2 comprising the amino acid sequence of SEQ ID NO: 22; and
(c) a VH CDR3 comprising the amino acid sequence of SEQ ID NO: 24.

15. A purified antibody or antibody fragment, wherein the antibody or the fragment (i) comprises a VH chain domain comprising three CDRs and a VL chain domain comprising three CDRs; and (ii) specifically binds human c-MPL, wherein the three CDRs of the VL chain domain comprise:
(a) a VL CDR1 comprising the amino acid sequence of SEQ ID NO: 2;
(b) a VL CDR2 comprising the amino acid sequence of SEQ ID NO: 4; and
(c) a VL CDR3 comprising the amino acid sequence of SEQ ID NO: 6.

16. A purified antibody or antibody fragment, wherein the antibody or the fragment specifically binds human c-MPL and comprises a heavy chain variable domain having at least 90% identity to the amino acid of SEQ ID NO: 40 and comprises a light chain variable domain having at least 90% identity to the amino acid sequence of SEQ ID NO: 38, wherein said antibody has the activity of antagonizing human-c-MPL.

17. A purified antibody or antibody fragment, wherein the antibody or the fragment comprises the heavy chain variable domain of SEQ ID NO: 40 and the light chain variable domain of SEQ ID NO: 38.

18. A purified/isolated antibody or fragment of an antibody, wherein the antibody or the fragment specifically binds to human c-MPL and comprises (A-2/mAb 1.75):
(a) a VH CDR1 having an amino acid sequence identical to or comprising 1, 2, or 3 amino acid residue substitutions relative to SEQ ID NO: 26;
(b) a VH CDR2 having an amino acid sequence identical to or comprising 1, 2, or 3 amino acid residue substitutions relative to SEQ ID NO: 28;
(c) a VH CDR3 having an amino acid sequence identical to or comprising 1, 2, or 3 amino acid residue substitutions relative to SEQ ID NO: 30;
(d) a VL CDR1 having an amino acid sequence identical to or comprising 1, 2, or 3 amino acid residue substitutions relative to SEQ ID NO: 8;
(e) a VL CDR2 having an amino acid sequence identical to or comprising 1, 2, or 3 amino acid residue substitutions relative to SEQ ID NO: 10; and
(f) a VL CDR3 having an amino acid sequence identical to or comprising 1, 2, or 3 amino acid residue substitutions relative to SEQ ID NO: 12.

19. A purified/isolated antibody or fragment of an antibody, wherein the antibody or the fragment specifically binds to human c-MPL and comprises:
(a) a VH CDR1 having the amino acid sequence of SEQ ID NO: 26;
(b) a VH CDR2 having the amino acid sequence of SEQ ID NO: 28;
(c) a VH CDR3 having the amino acid sequence of SEQ ID NO: 30;
(d) a VL CDR1 having the amino acid sequence of SEQ ID NO: 8;
(e) a VL CDR2 having the amino acid sequence of SEQ ID NO: 10; and
(f) a VL CDR3 having the amino acid sequence of SEQ ID NO: 12.

20. A purified antibody or antibody fragment, wherein the antibody or the fragment (i) comprises a VH chain domain comprising three CDRs and a VL chain domain comprising three CDRs; and (ii) specifically binds human c-MPL, wherein the three CDRs of the VH chain domain comprise:
(a) a VH CDR1 comprising the amino acid sequence of SEQ ID NO: 26;
(b) a VH CDR2 comprising the amino acid sequence of SEQ ID NO: 28; and
(c) a VH CDR3 comprising the amino acid sequence of SEQ ID NO: 30.

21. A purified antibody or antibody fragment, wherein the antibody or the fragment (i) comprises a VH chain domain comprising three CDRs and a VL chain domain comprising three CDRs; and (ii) specifically binds human c-MPL, wherein the three CDRs of the VL chain domain comprise:
(a) a VL CDR1 comprising the amino acid sequence of SEQ ID NO: 8;
(b) a VL CDR2 comprising the amino acid sequence of SEQ ID NO: 10; and
(c) a VL CDR3 comprising the amino acid sequence of SEQ ID NO: 12.

22. A purified antibody or antibody fragment, wherein the antibody or the fragment specifically binds human c-MPL and comprises a heavy chain variable domain having at least 90% identity to the amino acid of SEQ ID NO: 44 and comprises a light chain variable domain having at least 90% identity to the amino acid sequence of SEQ ID NO:42, wherein said antibody has the activity of antagonizing human-c-MPL.

23. A purified antibody or antibody fragment, wherein the antibody or the fragment comprises the heavy chain variable domain of SEQ ID NO: 44 and the light chain variable domain of SEQ ID NO:42.

24. A purified/isolated antibody or fragment of an antibody, wherein the antibody or the fragment specifically binds to human c-MPL and comprises (A-3/mAb 1.78):
(a) a VH CDR1 having an amino acid sequence identical to or comprising 1, 2, or 3 amino acid residue substitutions relative to SEQ ID NO: 32;
(b) a VH CDR2 having an amino acid sequence identical to or comprising 1, 2, or 3 amino acid residue substitutions relative to SEQ ID NO: 34;
(c) a VH CDR3 having an amino acid sequence identical to or comprising 1, 2, or 3 amino acid residue substitutions relative to SEQ ID NO: 36;
(d) a VL CDR1 having an amino acid sequence identical to or comprising 1, 2, or 3 amino acid residue substitutions relative to SEQ ID NO: 14;
(e) a VL CDR2 having an amino acid sequence identical to or comprising 1, 2, or 3 amino acid residue substitutions relative to SEQ ID NO: 16; and
(f) a VL CDR3 having an amino acid sequence identical to or comprising 1, 2, or 3 amino acid residue substitutions relative to SEQ ID NO: 18.

25. A purified/isolated antibody or fragment of an antibody, wherein the antibody or the fragment specifically binds to human c-MPL and comprises:
(a) a VH CDR1 having the amino acid sequence of SEQ ID NO:32;
(b) a VH CDR2 having the amino acid sequence of SEQ ID NO: 34;
(c) a VH CDR3 having the amino acid sequence of SEQ ID NO: 36;
(d) a VL CDR1 having the amino acid sequence of SEQ ID NO: 14;
(e) a VL CDR2 having the amino acid sequence of SEQ ID NO: 16; and
(f) a VL CDR3 having the amino acid sequence of SEQ ID NO: 18.

26. A purified antibody or antibody fragment, wherein the antibody or the fragment (i) comprises a VH chain domain comprising three CDRs and a VL chain domain comprising three CDRs; and (ii) specifically binds human c-MPL, wherein the three CDRs of the VH chain domain comprise:
(a) a VH CDR1 comprising the amino acid sequence of SEQ ID NO: 32;
(b) a VH CDR2 comprising the amino acid sequence of SEQ ID NO: 34; and
(c) a VH CDR3 comprising the amino acid sequence of SEQ ID NO: 36.

27. A purified antibody or antibody fragment, wherein the antibody or the fragment (i) comprises a VH chain domain comprising three CDRs and a VL chain domain comprising three CDRs; and (ii) specifically binds human c-MPL, wherein the three CDRs of the VL chain domain comprise:
(a) a VL CDR1 comprising the amino acid sequence of SEQ ID NO: 14;
(b) a VL CDR2 comprising the amino acid sequence of SEQ ID NO: 16 and
(c) a VL CDR3 comprising the amino acid sequence of SEQ ID NO: 18.

28. A purified antibody or antibody fragment, wherein the antibody or the fragment specifically binds human c-MPL and comprises a heavy chain variable domain having at least 90% identity to the amino acid of SEQ ID NO: 48 and comprises a light chain variable domain having at least 90% identity to the amino acid sequence of SEQ ID NO: 46, wherein said antibody has the activity of antagonizing human-c-MPL.

29. A purified antibody or antibody fragment, wherein the antibody or the fragment comprises the heavy chain variable domain of SEQ ID NO: 48 and the light chain variable domain of SEQ ID NO: 46.

30. A purified/isolated antibody or fragment of an antibody, wherein the antibody or the fragment specifically binds to human c-MPL and comprises (A-4/mAb1.111):
(a) a VH CDR1 having an amino acid sequence identical to or comprising 1, 2, or 3 amino acid residue substitutions relative to SEQ ID NO: 68;
(b) a VH CDR2 having an amino acid sequence identical to or comprising 1, 2, or 3 amino acid residue substitutions relative to SEQ ID NO: 70;
(c) a VH CDR3 having an amino acid sequence identical to or comprising 1, 2, or 3 amino acid residue substitutions relative to SEQ ID NO: 72;
(d) a VL CDR1 having an amino acid sequence identical to or comprising 1, 2, or 3 amino acid residue substitutions relative to SEQ ID NO: 50;
(e) a VL CDR2 having an amino acid sequence identical to or comprising 1, 2, or 3 amino acid residue substitutions relative to SEQ ID NO: 52; and
(f) a VL CDR3 having an amino acid sequence identical to or comprising 1, 2, or 3 amino acid residue substitutions relative to SEQ ID NO: 54.

31. A purified/isolated antibody or fragment of an antibody, wherein the antibody or the fragment specifically binds to human c-MPL and comprises:
(a) a VH CDR1 having the amino acid sequence of SEQ ID NO: 68;
(b) a VH CDR2 having the amino acid sequence of SEQ ID NO: 70
(c) a VH CDR3 having the amino acid sequence of SEQ ID NO: 72;
(d) a VL CDR1 having the amino acid sequence of SEQ ID NO: 50;
(e) a VL CDR2 having the amino acid sequence of SEQ ID NO: 52; and
(f) a VL CDR3 having the amino acid sequence of SEQ ID NO: 54.

32. A purified antibody or antibody fragment, wherein the antibody or the fragment (i) comprises a VH chain domain comprising three CDRs and a VL chain domain comprising three CDRs; and (ii) specifically binds human c-MPL, wherein the three CDRs of the VH chain domain comprise:
(a) a VH CDR1 comprising the amino acid sequence of SEQ ID NO: 68;
(b) a VH CDR2 comprising the amino acid sequence of SEQ ID NO: 70; and
(c) a VH CDR3 comprising the amino acid sequence of SEQ ID NO: 72.

33. A purified antibody or antibody fragment, wherein the antibody or the fragment (i) comprises a VH chain domain comprising three CDRs and a VL chain domain comprising three CDRs; and (ii) specifically binds human c-MPL, wherein the three CDRs of the VL chain domain comprise:
(a) a VL CDR1 comprising the amino acid sequence of SEQ ID NO: 50;
(b) a VL CDR2 comprising the amino acid sequence of SEQ ID NO: 52; and
(c) a VL CDR3 comprising the amino acid sequence of SEQ ID NO: 54.

34. A purified antibody or antibody fragment, wherein the antibody or the fragment specifically binds human c-MPL and comprises a heavy chain variable domain having at least 90% identity to the amino acid of SEQ ID NO: 88 and comprises a light chain variable domain having at least 90% identity to the amino acid sequence of SEQ ID NO: 86, wherein said antibody has the activity of antagonizing human-c-MPL.

35. A purified antibody or antibody fragment, wherein the antibody or the fragment comprises the heavy chain variable domain of SEQ ID NO: 88 and the light chain variable domain of SEQ ID NO: 86.

36. A purified/isolated antibody or fragment of an antibody, wherein the antibody or the fragment specifically binds to human c-MPL and comprises (A-5/mAb 1.36):
(a) a VH CDR1 having an amino acid sequence identical to or comprising 1, 2, or 3 amino acid residue substitutions relative to SEQ ID NO: 74;
(b) a VH CDR2 having an amino acid sequence identical to or comprising 1, 2, or 3 amino acid residue substitutions relative to SEQ ID NO: 76;
(c) a VH CDR3 having an amino acid sequence identical to or comprising 1, 2, or 3 amino acid residue substitutions relative to SEQ ID NO: 78;
(d) a VL CDR1 having an amino acid sequence identical to or comprising 1, 2, or 3 amino acid residue substitutions relative to SEQ ID NO: 56;
(e) a VL CDR2 having an amino acid sequence identical to or comprising 1, 2, or 3 amino acid residue substitutions relative to SEQ ID NO: 58; and
(f) a VL CDR3 having an amino acid sequence identical to or comprising 1, 2, or 3 amino acid residue substitutions relative to SEQ ID NO: 60.

37. A purified/isolated antibody or fragment of an antibody, wherein the antibody or the fragment specifically binds to human c-MPL and comprises:
(a) a VH CDR1 having the amino acid sequence of SEQ ID NO: 74;
(b) a VH CDR2 having the amino acid sequence of SEQ ID NO: 76;
(c) a VH CDR3 having the amino acid sequence of SEQ ID NO: 78;
(d) a VL CDR1 having the amino acid sequence of SEQ ID NO: 56;
(e) a VL CDR2 having the amino acid sequence of SEQ ID NO: 58; and
(f) a VL CDR3 having the amino acid sequence of SEQ ID NO: 60.

38. A purified antibody or antibody fragment, wherein the antibody or the fragment (i) comprises a VH chain domain comprising three CDRs and a VL chain domain comprising three CDRs; and (ii) specifically binds human c-MPL, wherein the three CDRs of the VH chain domain comprise:
(a) a VH CDR1 comprising the amino acid sequence of SEQ ID NO: 74;
(b) a VH CDR2 comprising the amino acid sequence of SEQ ID NO: 76; and
(c) a VH CDR3 comprising the amino acid sequence of SEQ ID NO: 78.

39. A purified antibody or antibody fragment, wherein the antibody or the fragment (i) comprises a VH chain domain comprising three CDRs and a VL chain domain comprising three CDRs; and (ii) specifically binds human c-MPL, wherein the three CDRs of the VL chain domain comprise:
(a) a VL CDR1 comprising the amino acid sequence of SEQ ID NO: 56;
(b) a VL CDR2 comprising the amino acid sequence of SEQ ID NO: 58; and
(c) a VL CDR3 comprising the amino acid sequence of SEQ ID NO: 60.

40. A purified antibody or antibody fragment, wherein the antibody or the fragment specifically binds human c-MPL and comprises a heavy chain variable domain having at least 90% identity to the amino acid of SEQ ID NO: 92 and comprises a light chain variable domain having at least 90% identity to the amino acid sequence of SEQ ID NO:90, wherein said antibody has the activity of antagonizing human-c-MPL.

41. A purified antibody or antibody fragment, wherein the antibody or the fragment comprises the heavy chain variable domain of SEQ ID NO: 92 and the light chain variable domain of SEQ ID NO:90.

42. A purified/isolated antibody or fragment of an antibody, wherein the antibody or the fragment specifically binds to human c-MPL and comprises (A-6/mAb 1.169):
(a) a VH CDR1 having an amino acid sequence identical to or comprising 1, 2, or 3 amino acid residue substitutions relative to SEQ ID NO: 80;
(b) a VH CDR2 having an amino acid sequence identical to or comprising 1, 2, or 3 amino acid residue substitutions relative to SEQ ID NO: 82;
(c) a VH CDR3 having an amino acid sequence identical to or comprising 1, 2, or 3 amino acid residue substitutions relative to SEQ ID NO: 84;
(d) a VL CDR1 having an amino acid sequence identical to or comprising 1, 2, or 3 amino acid residue substitutions relative to SEQ ID NO: 62;
(e) a VL CDR2 having an amino acid sequence identical to or comprising 1, 2, or 3 amino acid residue substitutions relative to SEQ ID NO: 64; and
(f) a VL CDR3 having an amino acid sequence identical to or comprising 1, 2, or 3 amino acid residue substitutions relative to SEQ ID NO: 66.

43. A purified/isolated antibody or fragment of an antibody, wherein the antibody or the fragment specifically binds to human c-MPL and comprises:
(a) a VH CDR1 having the amino acid sequence of SEQ ID NO:80;
(b) a VH CDR2 having the amino acid sequence of SEQ ID NO: 82;
(c) a VH CDR3 having the amino acid sequence of SEQ ID NO: 84;
(d) a VL CDR1 having the amino acid sequence of SEQ ID NO: 62;
(e) a VL CDR2 having the amino acid sequence of SEQ ID NO: 64; and
(f) a VL CDR3 having the amino acid sequence of SEQ ID NO: 66.

44. A purified antibody or antibody fragment, wherein the antibody or the fragment (i) comprises a VH chain domain comprising three CDRs and a VL chain domain comprising three CDRs; and (ii) specifically binds human c-MPL, wherein the three CDRs of the VH chain domain comprise:
(a) a VH CDR1 comprising the amino acid sequence of SEQ ID NO: 80;
(b) a VH CDR2 comprising the amino acid sequence of SEQ ID NO: 82; and
(c) a VH CDR3 comprising the amino acid sequence of SEQ ID NO: 84.

45. A purified antibody or antibody fragment, wherein the antibody or the fragment (i) comprises a VH chain domain comprising three CDRs and a VL chain domain comprising three CDRs; and (ii) specifically binds human c-MPL, wherein the three CDRs of the VL chain domain comprise:
(a) a VL CDR1 comprising the amino acid sequence of SEQ ID NO: 62;
(b) a VL CDR2 comprising the amino acid sequence of SEQ ID NO: 64 and
(c) a VL CDR3 comprising the amino acid sequence of SEQ ID NO: 66.

46. A purified antibody or antibody fragment, wherein the antibody or the fragment specifically binds human c-MPL and comprises a heavy chain variable domain having at least 90% identity to the amino acid of SEQ ID NO: 96 and comprises a light chain variable domain having at least 90% identity to the amino acid sequence of SEQ ID NO: 94, wherein said antibody has the activity of antagonizing human-c-MPL.

47. A purified antibody or antibody fragment, wherein the antibody or the fragment comprises the heavy chain variable domain of SEQ ID NO: 96 and the light chain variable domain of SEQ ID NO: 94.

48. The antibody of any of claims 1-47 wherein said antibody further binds cynomolgus MPL.

49. The antibody of any of claims 1-47 wherein said antibody does not compete for binding with rhuTPO.

50. The antibody of any of claims 1-49, wherein said antibody is selected from the group consisting of a human antibody, a humanized antibody, chimeric antibody, a monoclonal antibody, a polyclonal antibody, a recombinant antibody, an antigen-binding antibody fragment, a single chain antibody, a diabody, a triabody, a tetrabody, a Fab fragment, an F(fa′)x fragment, a domain antibody, an IgD antibody, an IgE antibody, and IgM antibody, and IgG1 antibody, and IgG2 antibody, and IgG3 antibody, and IgG4 antibody, and IgG4 antibody having at least one mutation in the hinge region.

51. The antibody of claim 50, wherein the antibody is a monoclonal antibody.

52. The antibody of claim 50, wherein the antibody is a chimeric antibody, a humanized antibody, or a fully human antibody.

53. A sterile composition comprising the antibody of any one of claims 1-52.

54. The composition of claim 53 further comprising a pharmaceutically acceptable carrier.

55. An isolated nucleic acid encoding the antibody of any of claims 1-52.

56. An expression vector comprising the nucleic acid of claim 55.

57. An isolated cell comprising the vector or nucleic acid of claim 55 or 56.

58. The isolated cell of claim 57, wherein the chromosome of the cell comprises the nucleic acid.

59. The isolated cell of claim 57, wherein the cell is a hybridoma.

60. A method of producing an antigen binding protein that specifically binds to the human c-MPL protein comprising incubating the host cell of claims 57-59 under conditions that allow it to express the antigen binding protein.

61. A method of inhibiting tumor cell proliferation in an animal, comprising administering to said animal a therapeutically effective dose of the antibody of any of claims 1-52.

62. A method of preventing, treating, or managing cancer in an animal in need thereof, said method comprising administering to said animal a dose of an effective amount of the composition of any of claim 53 or 54.

63. The method of any of claim 61 or 62, wherein said animal is human.

64. A method for treating, preventing or alleviating the symptoms of a c-MPL mediated disorder in a subject in need thereof comprising administering an effective amount of the antibody of any of claims 1-52.

65. A method for inhibiting c-MPL activity in a cell expressing c-MPL, comprising contacting the cell with the antibody of any of claims 1-52.

66. A method of determining the presence or absence of human c-MPL (TPO-R), comprising:
(a) contacting a sample with a human c-MPL-specific monoclonal antibody, or a functional fragment thereof, under conditions sufficient for binding, and
(b) measuring binding of said human c-MPL-specific monoclonal antibody, or functional fragment thereof, wherein binding of said human c-MPL-specific monoclonal antibody, or functional fragment thereof, indicates the presence of human c-MPL, and wherein an absence of binding of said human c-MPL-specific monoclonal antibody, or functional fragment thereof, indicates the absence of human c-MPL.

67. The method of claim 66, wherein said sample comprises a cell, cell lysate, tissue or organ.

68. The method of claim 66, wherein said human c-MPL-specific monoclonal antibody, or functional fragment thereof, comprises generally a Kd value less than or equal to a Kd selected from the group consisting of: 100 nM; 90; nM; 80 nM; 70 nM; 60 nM; 50 nM; 40 nM; 30 nM; 20 nM; 10 nM; 5 nM; 4 nM; 3 nM; 2 nM; 1 nM (e.g., 1000 pM); 900, pM; 800 pM; 700 pM; 600 pM; 500 pM; 400 pM; 300 pM; 200 pM; 100 pM; 50 pM; 25 pM; 20 pM; 15 pM; 10 pM; 5 pM; 3 pM or 1 pM.

69. The method of claim 66, wherein said human c-MPL-specific monoclonal antibody, or functional fragment thereof, inhibits binding to human c-MPL of a monoclonal antibody selected from the group consisting of 1.6; 1.75; 1.78; 1.111; 1.36; and 1.169.

70. The method of claim 66, wherein said human c-MPL-specific antibody, or functional fragment thereof, is selected from the monoclonal antibodies consisting of 1.6; 1.75; 1.78; 1.111; 1.36; and 1.169.

71. The method of claim 66, wherein said functional fragment comprises an antibody binding fragment selected from Fd, Fv, Fab, F(ab′), F(ab)₂, F(ab′)₂ and scFv.

72. A method of determining susceptibility of a cell to c-MPL-mediated proliferation, comprising:
(a) contacting a cellular sample with a human c-MPL-specific monoclonal antibody, or functional fragment thereof, under conditions sufficient for binding, and
(b) measuring the binding of said human c-MPL-specific monoclonal antibody, or functional fragment thereof, wherein the presence of human c-MPL indicates susceptibility of said cellular sample to human c-MPL-mediated proliferation.

73. The method of claim 72 wherein said cellular sample comprises a cell, tissue or organ.

74 The method of claim 72, wherein said human c-MPL-specific monoclonal antibody, or functional fragment thereof, comprises generally a Kd value less than or equal to a Kd selected from the group consisting of: 100 nM; 90; nM; 80 nM; 70 nM; 60 nM; 50 nM; 40 nM; 30 nM; 20 nM; 10 nM; 5 nM; 4 nM; 3 nM; 2 nM; 1 nM (e.g., 1000 pM); 500 pM; 400 pM; 300 pM; 200 pM; 100 pM; 50 pM; 25 pM; 20 pM; 15 pM; 10 pM; 5 pM; 3 pM or 1 pM.

75. The method of claim 72, wherein said human c-MPL-specific monoclonal antibody, or functional fragment thereof, inhibits binding to human c-MPL of a monoclonal antibody selected from the group consisting of 1.6; 1.75; 1.78; 1.111; 1.36; and 1.169.

76. The method of claim 72 wherein said human c-MPL-specific antibody, or functional fragment thereof, is selected from the monoclonal antibodies consisting of 1.6; 1.75; 1.78; 1.111; 1.36; and 1.169.

77. The method of claim 72, wherein said functional fragment comprises an antibody binding fragment selected from Fd, Fv, Fab, F(ab′), F(ab)₂, F(ab′)₂ and scFv.

78. A kit for determining the presence or absence of human c-MPL, comprising:
(a) a monoclonal antibody having specific binding activity to human c-MPL, or a functional fragment thereof, and
(b) a detection reagent.

79. The kit of claim 77, wherein said antibody, or functional fragment thereof, inhibits the binding to human c-MPL of a monoclonal antibody selected from the group consisting of 1.6; 1.75; 1.78; 1.111; 1.36; and 1.169.

80. The kit of claim 78, wherein said antibody, or functional fragment thereof, is selected from the monoclonal antibodies consisting of 1.6; 1.75; 1.78; 1.111; 1.36; and 1.169.

81. The kit of claim 78, wherein said functional fragment comprises an antibody binding fragment selected from Fd, Fv, Fab, F(ab′), F(ab)₂, F(ab′)₂ and scFv.

82. The kit of claim 78, further comprising a polypeptide having a human c-MPL extracellular domain, a cell line expressing human c-MPL.

83. The kit of claim 78, further comprising an ancillary reagent.

84. The kit of claim 78, further comprising a c-MPL agonist.

85. The kit of claim 84, wherein said c-MPL agonist comprises TPO.

86. An antibody that binds to the c-MPL epitope corresponding to -PWQDGPK- (SEQ ID NO: 97).

### BRIEF DESCRIPTION OF THE FIGURES

FIG. 1. Differential expression of c-Mpl mRNA in hematopoietic cell lines. A: Cell lysates were analyzed by QuantiGene branched DNA assay using DNA probe sets that bind to either the intracellular (black bars) or extracellular (white bars) regions of human c-Mpl mRNA and the signal levels then normalized against the level of expression of the human housekeeping gene cyclophilin B (PPIB). Samples not containing cell lysate were used to set the background “noise” depicted by the horizontal dash line. B: In murine cell lines, cell lysates were hybridized with DNA probe sets that detect the extracellular regions of human c-Mpl mRNA or the mouse housekeeping gene cyclophilin A (PPIA). Results shown are from representative experiments with each value corresponding to the mean±SD of triplicate samples.

FIG. 2. Flow cytometric analysis of anti-c-Mpl Ab binding to nontransfected 32D cells (parental) or 32D cells engineered to express high levels of c-Mpl protein (32D-hu-c-Mpl). Dashed lines depict cells not stained with antibody, solid lines depict cells stained with the appropriate isotype control antibody followed by PE-conjugated secondary antibody, and the shaded histograms depict cells stained with the indicated anti-c-Mpl Abs followed by PE-conjugated secondary antibody.

FIG. 3. Flow cytometric analysis of anti-c-Mpl Ab binding to c-Mpl+ CMK and c-Mpl-K562 cells. Dashed lines depict cells not stained with antibody, solid lines depict cells stained with the appropriate isotype control antibody followed by PE-conjugated secondary antibody, and the shaded histograms depict cells stained with the indicated anti-c-Mpl Abs followed by PE-conjugated secondary antibody.

FIG. 4. Flow cytometric analysis of the binding anti-c-Mpl Abs 1.6, 1.75 and BAH-1 to three c-Mpl+ cell lines (panel A) and five c-Mpl− cell lines plus KG-1 cells (panel B). Dashed lines depict cells not stained with antibody, solid lines depict cells stained with PE-conjugated secondary antibody only, and the shaded histograms depict cells stained with the indicated anti-c-Mpl Abs followed by PE-conjugated secondary antibody.

### DETAILED DESCRIPTION OF THE INVENTION

The present invention relates to antigen binding proteins such as antibodies that specifically bind to the human c-MPL receptor (also known as the human TPO-receptor). These include antigen binding proteins that inhibit or block the binding of TPO to human c-MPL, and reduce TPO signalling through the receptor. In one embodiment, provided are human antibodies including antagonistic antibodies capable of binding to the human c-mpl receptor. The antigen binding proteins are useful for treating diseases that result from increased or uninhibited (e.g., non-antagonized) c-MPL activity, as well as in diagnostic methods.

The present invention further provides compositions, kits, and methods relating to antigen binding proteins that specifically bind to the human c-MPL receptor. Also provided are nucleic acid molecules, and derivatives and fragments thereof, comprising a sequence of polynucleotides that encode all or a portion of an antigen binding protein that binds to the c-MPL receptor, such as a nucleic acid encoding all or part of an anti-c-MPL receptor antibody, antibody fragment, or antibody derivative. The present invention further provides vectors and plasmids comprising such nucleic acids, and cells or cell lines comprising such nucleic acids and/or vectors and plasmids. The provided methods include, for example, methods of making, identifying, or isolating antigen binding proteins that bind to human c-MPL, such as anti-c-MPL antibodies, methods of determining whether an antigen binding protein binds to c-MPL, methods of making compositions, such as pharmaceutical compositions, comprising an antigen binding protein that binds to human c-MPL, and methods for administering an antigen binding protein that binds c-MPL to a subject, for example, methods for treating a condition mediated by c-MPL, and for modulating a biological activity associated with c-MPL signalling in vivo or in vitro.

### DEFINITIONS

Polynucleotide and polypeptide sequences are indicated using standard one- or three-letter abbreviations. Unless otherwise indicated, polypeptide sequences have their amino termini at the left and their carboxy termini at the right, and single-stranded nucleic acid sequences, and the top strand of double-stranded nucleic acid sequences, have their 5′ termini at the left and their 3′ termini at the right. A particular polypeptide or polynucleotide sequence also can be described by explaining how it differs from a reference sequence. Polynucleotide and polypeptide sequences of particular light and heavy chain variable domains are designated L1 (“light chain variable domain 1”) and H1 (“heavy chain variable domain 1”). Antigen binding proteins or antibodies comprising a light chain and heavy chain are indicated by combining the name of the light chain and the name of the heavy chain variable domains. For example, “L2H3,” indicates, for example, an antibody comprising the light chain variable domain of L2 and the heavy chain variable domain of H3.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

The following terms, unless otherwise indicated, shall be understood to have the following meanings: The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A neutralizing antibody or an inhibitory antibody when used in reference to an anti-c-MPL-specific monoclonal antibody of the invention refers to monoclonal antibody that inhibits the binding of TPO to c-MPL, preferably human c-MPL, when an excess of the anti-c-MPL-specific monoclonal antibody reduces the amount of TPO bound to c-MPL (TPO-R). Binding inhibition can occur by at least 10%, particularly by at least about 20%. In various specific examples, the monoclonal antibody can reduce the amount of TPO bound to c-MPL (preferably human) by, for example, at least 30%, 40%, 50%, 60%, 70%, 75%, 80%, 85%, 90%, 95%, 97%, 99%, and 99.9%. The binding reduction may be measured by any means known to one of ordinary skill in the art, for example, as measured in an in vitro competitive binding assay.

An “antagonistic” antibody refers to an antibody that inhibits the activity of c-MPL when added to a cell, tissue or organism expressing c-MPL receptor, preferably the human c-MPL. Diminution in activity can be by at least about 5%, particularly by at least about 10%, more particularly, by at least about 15% or more, compared to the level of c-MPL (TPO-R) activity in the presence of TPO alone. In various specific examples, the c-MPL-specific monoclonal antibodies of the invention can inhibit the c-MPL, preferably human, activity by at least about 20%, 30%, 40%, 50%, 60%, 70%, 80%, 90% or 100%.

The terms “peptide” “polypeptide” and “protein” each refers to a molecule comprising two or more amino acid residues joined to each other by peptide bonds. These terms encompass, e.g., native and artificial proteins, protein fragments and polypeptide analogs (such as muteins, variants, and fusion proteins) of a protein sequence as well as post-translationally, or otherwise covalently or non-covalently, modified proteins. A peptide, polypeptide, or protein may be monomeric or polymeric.

The term “polypeptide fragment” as used herein refers to a polypeptide that has an amino-terminal and/or carboxy-terminal deletion as compared to a corresponding full-length protein. Fragments can be, for example, at least 5, 6, 7, 8, 9, 10, 11, 12, 13, 14, 15, 20, 50, 70, 80, 90, 100, 150 or 200 amino acids in length. Fragments can also be, for example, at most 1,000, 750, 500, 250, 200, 175, 150, 125, 100, 90, 80, 70, 60, 50, 40, 30, 20, 15, 14, 13, 12, 11, or 10 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

Polypeptides of the invention include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (4) confer or modify other physicochemical or functional properties. Analogs include muteins of a polypeptide. For example, single or multiple amino acid substitutions (e.g., conservative amino acid substitutions) may be made in the naturally occurring sequence (e.g., in the portion of the polypeptide outside the domain(s) forming intermolecular contacts). A “conservative amino acid substitution” is one that does not substantially change the structural characteristics of the parent sequence (e.g., a replacement amino acid should not tend to break a helix that occurs in the parent sequence, or disrupt other types of secondary structure that characterize the parent sequence or are necessary for its functionality). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

The present invention also provides non-peptide analogs of c-MPL antigen binding proteins. Non-peptide analogs are commonly used to provide drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al. J. Med. Chem. 30:1229 (1987), which are incorporated herein by reference. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a desired biochemical property or pharmacological activity), such as a human antibody, but have one or more peptide linkages optionally replaced by a linkage selected from the group consisting of: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference), for example, by adding internal cysteine residues capable of forming intramolecular disulfide bridges which cyclize the peptide.

A “variant” of a polypeptide (e.g., an antibody) comprises an amino acid sequence wherein one or more amino acid residues are inserted into, deleted from and/or substituted into the amino acid sequence relative to another polypeptide sequence. Variants of the invention include fusion proteins.

A “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising two full-length heavy chains and two full-length light chains, derivatives, variants, fragments, and muteins thereof, examples of which are described below.

An “antigen binding protein” is a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129 (2003); Roque et al., Biotechnol. Prog. 20:639-654 (2004). In addition, peptide antibody mimetics (“PAMs”) can be used, as well as scaffolds based on antibody mimetics utilizing fibronection components as a scaffold.

An antigen binding protein can have, for example, the structure of a naturally occurring immunoglobulin. An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

Naturally occurring immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4. The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991.

An “antibody” refers to an intact immunoglobulin or to an antigen binding portion thereof that competes with the intact antibody for specific binding, unless otherwise specified. Antigen binding portions may be produced by recombinant DNA techniques or by enzymatic or chemical cleavage of intact antibodies. Antigen binding portions include, inter alia, Fab, Fab′, F(ab′)₂, Fv, domain antibodies (dAbs), fragments including complementarity determining regions (CDRs), single-chain antibodies (scFv), chimeric antibodies, diabodies, triabodies, tetrabodies, and polypeptides that contain at least a portion of an immunoglobulin that is sufficient to confer specific antigen binding to the polypeptide.

A Fab fragment is a monovalent fragment having the V_{L}, V_{H}, C_{L} and C_{H}1 domains; a F(ab′)₂ fragment is a bivalent fragment having two Fab fragments linked by a disulfide bridge at the hinge region; a Fd fragment has the V_{H} and C_{H}1 domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. Nos. 6,846,634, 6,696,245, US App. Pub. No. 05/0202512, 04/0202995, 04/0038291, 04/0009507, 03/0039958, Ward et al., Nature 341:544-546 (1989)).

A single-chain antibody (scFv) is an antibody in which a V_{L} and a V_{H} region are joined via a linker (e.g., a synthetic sequence of amino acid residues) to form a continuous protein chain wherein the linker is long enough to allow the protein chain to fold back on itself and form a monovalent antigen binding site (see, e.g., Bird et al., Science 242:423-26 (1988) and Huston et al., 1988, Proc. Natl. Acad. Sci. USA 85:5879-83 (1988)). Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48 (1993), and Poljak et al., Structure 2:1121-23 (1994)). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

Complementarity determining regions (CDRs) and framework regions (FR) of a given antibody may be identified using the system described by Kabat et al. in Sequences of Proteins of Immunological Interest, 5th Ed., US Dept. of Health and Human Services, PHS, NIH, NIH Publication no. 91-3242, 1991. One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

An antigen binding protein may have one or more binding sites. If there is more than one binding site, the binding sites may be identical to one another or may be different. For example, a naturally occurring human immunoglobulin typically has two identical binding sites, while a “bispecific” or “bifunctional” antibody has two different binding sites.

The term “human antibody” includes all antibodies that have one or more variable and constant regions derived from human immunoglobulin sequences. In one embodiment, all of the variable and constant domains are derived from human immunoglobulin sequences (a fully human antibody). These antibodies may be prepared in a variety of ways, examples of which are described below, including through the immunization with an antigen of interest of a mouse that is genetically modified to express antibodies derived from human heavy and/or light chain-encoding genes.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. In one embodiment, one or more of the CDRs are derived from a human anti-c-MPL antibody. In another embodiment, all of the CDRs are derived from a human anti-c-MPL antibody. In another embodiment, the CDRs from more than one human anti-c-MPL antibodies are mixed and matched in a chimeric antibody. For instance, a chimeric antibody may comprise a CDR1 from the light chain of a first human anti-c-MPL antibody, a CDR2 and a CDR3 from the light chain of a second human anti-c-MPL antibody, and the CDRs from the heavy chain from a third anti-c-MPL antibody. Further, the framework regions may be derived from one of the same anti-c-MPL antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody or antibodies from another species or belonging to another antibody class or subclass. Also included are fragments of such antibodies that exhibit the desired biological activity (i.e., the ability to specifically bind the human c-MPL receptor).

A “neutralizing antibody” or “inhibitory antibody” or “antagonistic antibody” refers to an antibody that inhibits the binding of TPO to the human c-MPL receptor, and/or inhibits or reduces TPO-mediated signalling, as determined, for example, by the 32D-hyu-c-MPL Cell Proliferation assay described in Example 4 below. The inhibition need not be complete and may be, in one embodiment, reduced binding or signalling by at least 20%. In further embodiments, the reduction in binding or signalling is at least 30%, 40%, 50%, 60%, 70%, 80%, 85%, 90%, 95%, 97%, 99% and 99.9%.

Fragments or analogs of antibodies can be readily prepared by those of ordinary skill in the art following the teachings of this specification and using techniques well-known in the art. Preferred amino- and carboxy-termini of fragments or analogs occur near boundaries of functional domains. Structural and functional domains can be identified by comparison of the nucleotide and/or amino acid sequence data to public or proprietary sequence databases. Computerized comparison methods can be used to identify sequence motifs or predicted protein conformation domains that occur in other proteins of known structure and/or function. Methods to identify protein sequences that fold into a known three-dimensional structure are known. See, e.g., Bowie et al., Science 253:164 (1991).

A “CDR grafted antibody” is an antibody comprising one or more CDRs derived from an antibody of a particular species or isotype and the framework of another antibody of the same or different species or isotype.

A “multi-specific antibody” is an antibody that recognizes more than one epitope on one or more antigens. A subclass of this type of antibody is a “bi-specific antibody” which recognizes two distinct epitopes on the same or different antigens.

An antigen binding protein including an antibody “specifically binds” to an antigen, such as the human c-MPL receptor if it binds to the antigen with a high binding affinity as determined by a dissociation constant (Kd, or corresponding Kb, as defined below) value of 10⁻⁷ M or less. An antigen binding protein that specifically binds to the human c-MPL receptor may be able to bind to c-MPL receptors from other species as well with the same or different affinities.

An “epitope” is the portion of a molecule that is bound by an antigen binding protein (e.g., by an antibody). An epitope can comprise non-contiguous portions of the molecule (e.g., in a polypeptide, amino acid residues that are not contiguous in the polypeptide's primary sequence but that, in the context of the polypeptide's tertiary and quaternary structure, are near enough to each other to be bound by an antigen binding protein).

The “percent identity” of two polynucleotide or two polypeptide sequences is determined by comparing the sequences using the GAP computer program (a part of the GCG Wisconsin Package, version 10.3 (Accelrys, San Diego, Calif.)) using its default parameters.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In one embodiment, the nucleic acid molecules of the invention comprise a contiguous open reading frame encoding an antibody, or a fragment, derivative, mutein, or variant thereof, of the invention.

Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

A “vector” is a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology Methods in Enzymology 185, Academic Press, San Diego, Calif. and Baron et al., 1995, Nucleic Acids Res. 23:3605-06.

A “host cell” is a cell that can be used to express a nucleic acid, e.g., a nucleic acid of the invention. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Typically, a host cell is a cultured cell that can be transformed or transfected with a polypeptide-encoding nucleic acid, which can then be expressed in the host cell. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The phrase “biologically active” and “desired biological activity” for the purposes herein means (1) having the ability to antagonize, neutralize or block the human c-MPL receptor in at least one type of mammalian c-MPL+ cell in vivo or ex vivo.

The terms “treating,” “treatment,” and “therapy” as used herein refer to curative therapy, prophylactic therapy, and preventative therapy.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, blastoma, gastrointestinal cancer, renal cancer, pancreatic cancer, glioblastoma, neuroblastoma, cervical cancer, ovarian cancer, liver cancer, stomach cancer, bladder cancer, hepatoma, breast cancer, colon cancer, colorectal cancer, endometrial carcinoma, salivary gland carcinoma, kidney cancer, liver cancer, prostate cancer, vulval cancer, thyroid cancer, hepatic carcinoma and various types of head and neck cancer. Other particular examples of such cancers include chronic myelogenous leukemia (CML), acute myelogenous leukemia (AML), chronic myelomonocytic leukemia, juvenile myelomonocytic leukemia, systemic mastocytosis, hypereosinophilic syndrome, chronic neutrophilic leukemia, chronic eosinophilic leukemia, chronic basophilic leukemia, and the like.

The term “mammal” as used herein refers to any mammal classified as a mammal, including humans, cows, horses, dogs and cats. In a preferred embodiment of the invention, the mammal is a human.

As used herein, the term “c-MPL” refers to the cognate receptor for the thrombopoietin (TPO) protein. TPO and its cognate receptor, c-Mpl, are primary physiological regulators of megakaryopoiesis and play important roles in platelet production and hemostasis (Battinelli et al., Curr Opin Hematol (2007)14:419-426; Deutsch et al., Br J Haematol (2006)134:453-466; Kaushansky et al., J Clin Invest (2005)115:3339-3347). Human c-Mpl (set forth herein as SEQ ID NO: 99) s a 635 amino acid transmembrane protein and is a member of the single-chain, type I cytokine receptor subfamily of hematopoietic growth factor receptors that also includes the receptors for erythropoietin (Epo), granulocyte colony-stimulating factor (G-CSF), growth hormone (GH), and prolactin (PL) (Boulay et al., Immunity (2003)19:159-163). These receptors do not possess an intracellular kinase domain and require ligand-induced homodimerization followed by the association and activation of Janus kinases (JAKs) for signal transduction. Binding of TPO to c-Mpl leads to phosphorylation of receptor-bound JAK2 and the subsequent activation of several downstream signaling pathways, including the signal transducers and activators of transcription (STAT) pathway, the mitogen-activated protein kinase (MAPK) pathways involving both the extracellular signal-regulated kinases (ERK) and p38 kinases, and the phosphatidylinositol-3-kinase (PI3K) pathway (Kaushansky et al., J Clin Invest (2005)115:3339-3347).

The N-terminal 490 amino acid extracellular domain (ECD) of human c-Mpl contains two cytokine receptor modules (CRM). Each CRM is approximately 200 amino acids in length and contains four conserved cysteines in the N-terminal region and a pentapeptide WSXWS motif near the C-terminal end (Kaushansky et al., Oncogene (2002)21:3359-3367). Biochemical studies suggest that TPO binds to the distal CRM that plays an inhibitory role in c-Mpl signaling as the deletion of this CRM results in c-Mpl auto-activation (Sabath et al., Blood (1999)94:365-367). The 122 residue cytoplasmic domain of c-Mpl comprises two 60 amino acid regions. The membrane-proximal region contains two conserved sequence motifs, box 1 and box 2, and is essential for signal transduction and for cell surface expression (Morita et al., FEBS Lett (1996)395:228-234); Luoh et al., Mol Cell Biol (2000)20:507-515; Tong et al., J Biol. Chem. (2006)281:38930-38940. The distal 60 amino acid region is not required for megakaryopoiesis or platelet production under normal circumstances but appears to be important in maintaining platelet levels in situations of acute stress (Luoh et al., Mol Cell Biol. (2000)20:507-515).

Both genetic and biochemical studies have demonstrated that human c-Mpl is expressed primarily in hematopoietic tissues including hematopoietic stem and progenitor cells, megakaryocytes (MK), and platelets (Methia et al., Blood (1993)82:1395-1401; Graf et al., Leuk Res (1996)20:831-838; Solar et al., Blood (1998)92:4-10; Debili al., Blood (1995)85:391-401). Using Northern blotting and RT-PCR methodologies, Methia et al., showed the presence of c-Mpl transcripts in CD34+ cells, MK, and platelets in 1993 (Methia et al., Blood (1993)82:1395-1401).

In one embodiment, the antigen binding agents of the present invention may be selected to bind to membrane-bound c-MPL receptors as expressed on cells, and inhibit or block TPO signalling through the c-MPL receptor. In another embodiment, the antigen binding agents of the present invention specifically bind to the human c-MPL receptor. In a further embodiment, the antigen binding proteins binding to the human c-MPL receptor may also bind to the c-MPL receptors of other species. The Examples below provide one method of generating fully human antibodies which bind to human membrane-bound c-MPL receptors, and in a further embodiment, bind to c-MPL receptors of other species.

The polynucleotide and polypeptide sequences for several species of the c-MPL receptor are known. Table 1A presents sequences for human c-MPL.

TABLE 1A
MPL RECEPTOR SEQUENCES

Human (Homo sapiens) polynucleotides (SEQ ID NO: 98)
accession no. AH003655.1 GI: 1546807

| | |
|---|---|
| 1 | atgccctcct gggccctctt catggtcacc tcctgcctcc tcctggcccc tcaaaacctg |
| 61 | gcccaagtca gcagccaaga tgtctccttg ctggcatcag actcagagcc cctgaagtgt |
| 121 | ttctcccgaa catttgagga cctcacttgc ttctgggatg aggaagaggc agcgcccagt |
| 181 | gggacatacc agctgctgta tgcctacccg cgggagaagc cccgtgcttg ccccctgagt |
| 241 | tcccagagca tgccccactt tggaacccga tacgtgtgcc agtttccaga ccaggaggaa |
| 301 | gtgcgtctct tctttccgct gcacctctgg gtgaagaatg tgttcctaaa ccagactcgg |
| 361 | actcagcgag tcctctttgt ggacagtgta ggcctgccgg ctccccccag tatcatcaag |
| 421 | gccatgggtg ggagccagcc aggggaactt cagatcagct gggaggagcc agctccagaa |
| 481 | atcagtgatt tcctgaggta cgaactccgc tatggcccca gagatcccaa gaactccact |
| 541 | ggtcccacgg tcatacagct gattgccaca gaaacctgct gccctgctct gcagaggcct |
| 601 | cactcagcct ctgctctgga ccagtctcca tgtgctcagc ccacaatgcc ctggcaagat |
| 661 | ggaccaaagc agacctcccc aagtagagaa gcttcagctc tgacagcaga gggtggaagc |
| 721 | tgcctcatct caggactcca gcctggcaac tcctactggc tgcagctgcg cagcgaacct |
| 781 | gatgggatct ccctcggtgg ctcctgggga tcctggtccc tccctgtgac tgtggacctg |
| 841 | cctggagatg cagtggcact tggactgcaa tgctttacct tggacctgaa gaatgttacc |
| 901 | tgtcaatggc agcaacagga ccatgctagc tcccaaggct tcttctacca cagcagggca |
| 961 | cggtgctgcc ccagagacag gtaccccatc tgggagaact gcgaagagga agagaaaaca |
| 1021 | aatccaggac tacagacccc acagttctct cgctgccact tcaagtcacg aaatgacagc |
| 1081 | attattcaca tccttgtgga ggtgaccaca gccccgggta ctgttcacag ctacctgggc |
| 1141 | tcccctttct ggatccacca ggctgtgcgc ctccccaccc caaacttgca ctggagggag |
| 1201 | atctccagtg ggcatctgga attggagtgg cagcacccat cgtcctgggc agcccaagag |
| 1261 | acctgttatc aactccgata cacaggagaa ggccatcagg actggaaggt gctggagccg |
| 1321 | cctctcgggg cccgaggagg gaccctggag ctgcgcccgc gatctcgcta ccgtttacag |
| 1381 | ctgcgcgcca ggctcaacgg ccccacctac caaggtccct ggagctcgtg gtcggaccca |
| 1441 | actagggtgg agaccgccac cgagaccgcc tggatctcct tggtgaccgc tctgcatcta |
| 1501 | gtgctgggcc tcagcgccgt cctgggcctg ctgctgctga ggtggcagtt tcctgcacac |
| 1561 | tacaggagac tgaggcatgc cctgtggccc tcacttccag acctgcaccg ggtcctaggc |
| 1621 | cagtacctta gggacactgc agccctgagc ccgcccaagg ccacagtctc agatacctgt |
| 1681 | gaagaagtgg aacccagcct ccttgaaatc ctccccaagt cctcagagag gactcctttg |
| 1741 | cccctgtgtt cctcccaggc ccagatggac taccgaagat tgcagccttc ttgcctgggg |
| 1801 | accatgcccc tgtctgtgtg cccacccatg gctgagtcag ggtcctgctg taccacccac |
| 1861 | attgccaacc attcctacct accactaagc tattggcagc agccttga |

Human (Homo sapiens) amino acid (SEQ ID NO: 99)
accession no. AAB08424.1 GI: 1546808

| | |
|---|---|
| 1 | mpswalfmvt sclllapqnl aqvssqdvsl lasdseplkc fsrtfedltc fwdeeeaaps |
| 61 | gtyqllyayp rekpracpls sqsmphfgtr yvcqfpdqee vrlffplhlw vknvflnqtr |
| 121 | tqrvlfvdsv glpappsiik amggsqpgel qisweepape isdflryelr ygprdpknst |
| 181 | gptviqliat etccpalqrp hsasaldqsp caqptmpwqd gpkqtspsre asaltaeggs |
| 241 | clisglqpgn sywlqlrsep dgislggswg swslpvtvdl pgdavalglq cftldlknvt |
| 301 | cqwqqqdhas sqgffyhsra rccprdrypi wenceeeekt npglqtpqfs rchfksrnds |
| 361 | iihilvevtt apgtvhsylg spfwihqavr lptpnlhwre issghlelew qhpsswaaqe |
| 421 | tcyqlrytge ghqdwkvlep plgarggtle lrprsryrlq lrarlngpty qgpwsswsdp |
| 481 | trvetateta wislvtalhl vlglsavlgl lllrwqfpah yrrlrhalwp slpdlhrvlg |
| 541 | qylrdtaals ppkatvsdtc eevepsllei lpkssertpl plcssqaqmd yrrlqpsclg |
| 601 | tmplsvcppm aesgscctth ianhsylpls ywqqp |

### Antigen Binding Proteins

In one aspect, the present invention provides antigen binding proteins (e.g., antibodies, antibody fragments, antibody derivatives, antibody muteins, and antibody variants), that specifically bind to the human c-MPL receptor. In one embodiment the antigen binding protein is a human anti-c-MPL or a murine-anti-human c-MPL antibody.

In particular embodiments, c-MPL-specific monoclonal antibodies of the invention include monoclonal antibodies that exhibit c-MPL (or TPO-receptor) antagonistic activity. An antagonist of c-MPL receptor activity refers to a molecule that decreases at least one function or activity of c-MPL receptor when bound or stimulated by ligand. Such functions of the c-MPL receptor have are well know in the art and can include, for example, stimulation of cell proliferation, differentiation, migration and/or cell survival. Accordingly, c-MPL-specific monoclonal antibodies having TPO-R (c-MPL) antagonist activity decrease, reduce or prevent one or more of the above functions or activities of TPO-R exemplified herein, such as set forth in the Indication section hereinbelow. Other functions or activities of c-MPL also can be reduced or inhibited by the antagonistic c-MPL-specific monoclonal antibodies of the invention. Given the teachings and guidance provided herein, those skilled in the art will be able to make and identify a wide range of c-MPL-specific monoclonal antibodies exhibiting different antagonistic activities.

Antigen binding proteins in accordance with the present invention include antigen binding proteins that specifically bind to the human c-MPL receptor. In particular embodiments, the antigen binding proteins are human or murine-anti-human antibodies that specifically bind the c-MPL receptor, and inhibit signalling through the c-MPL receptor. In a further embodiment, the antigen binding proteins in accordance with the present invention include antigen binding proteins that specifically bind to the human c-MPL receptor and inhibit TPO-mediated signalling through the c-MPL receptor. In one embodiment, the IC50 value of the antigen binding protein is 90 nM or less. In other embodiments, the IC50 value of the antigen binding protein is less than or equal to: 80 nM; 70 nM; 60 nM; 50 nM; 40 nM; 30 nM; 20 nM; 10 nM; 5 nM; 4 nM; 3 nM; 2 nM; 1 nM (e.g., 1000 pM); 900, pM; 800 pM; 700 pM; 600 pM; 500 pM; 400 pM; 300 pM; 200 pM; 100 pM; 50 pM; 25 pM; 20 pM; 15 pM; 10 pM; 5 pM; 3 pM or 1 pM. In another aspect, the antigen binding proteins specifically bind the c-MPL receptor, inhibit TPO-mediated signalling, and exhibit therapeutic biological effects, such as inhibiting cancerous cell growth in a human patient or in animal models. In another embodiment, the antigen binding proteins are human antibodies that specifically bind to the human c-MPL receptor, inhibit c-MPL mediated signalling through the c-MPL receptor, and are capable of inhibiting hematopoietic cell proliferation and/or differentiation or treating cancer, such as leukemia and the like, in a human patient or in animal models.

In one embodiment, the antigen binding protein (e.g., antibody) comprises sequences that each independently differ by 5, 4, 3, 2, 1, or 0 single amino acid additions, substitutions, and/or deletions from a CDR sequence of A1-A6 in Table 2 below. As used herein, a CDR sequence that differs by no more than a total of, for example, four amino acid additions, substitutions and/or deletions from a CDR sequence shown in Table 2 below refers to a sequence with 4, 3, 2, 1 or 0 single amino acid additions, substitutions, and/or deletions compared with the sequences shown in Table 2.

The light chain CDRs of antigen binding proteins (antibodies) A1-A6 and the heavy chain CDRs of exemplary antigen binding proteins (antibodies) A1-A6 are shown below in Table 2. A-1 to A6 corresponds to L1 to L6 below, and H1 to H6 below. Also shown are polynucleotide sequences which encode the amino acid sequences of the CDRs.

TABLE 1B
ANTI-C-MPL ANTIBODIES

| Antibody | Corresponding Nomenclature |
|---|---|
| mAb 1.6 | A1, L1, H1 |
| mAb 1.75 | A2, L2, H2 |
| mAb 1.78 | A3, L3, H3 |
| mAb 1.111 | A4, L4, H4 |
| mAb 1.36 | A5, L5, H5 |
| mAb 1.169 | A6, L6, H6 |

TABLE 2
LIGHT CHAINS L1 TO L3

| mAb | CDR 1 | CDR 2 | CDR 3 |
|---|---|---|---|
| A-1 | ATGGCAAGTGAGGACATTTATATT | GCTGCAACCAGTTTGGAAACT | CAACAGTATTGGAGTTCTCCGTGG |
| NA | CGCTTAGCC (SEQ ID NO: 1) | (SEQ ID NO: 3) | ACG (SEQ ID NO: 5) |
| AA | M A S E D I Y I | A A T S L E T | Q Q Y W S S P W |
| | R L A (SEQ ID NO: 2) | (SEQ ID NO: 4) | T (SEQ ID NO: 6) |
| A-2 | AGTGCCAGCTCAAGTGTAAGTTAC | CGCACATCCAACCTGGCTTCT | CAGCAGTATCATAGTTACCCAACC |
| NA | ATGTAC (SEQ ID NO: 7) | (SEQ ID NO: 9) | ACG (SEQ ID NO: 11) |
| AA | S A S S S V S Y | R T S N L A S | Q Q Y H S Y P T |
| | M Y (SEQ ID NO: 8) | (SEQ ID NO: 10) | T (SEQ ID NO: 12) |
| A-3 | AGGTCTGATAAGAGTCTCCTGCAT | CGGATGTCCAACCTTGCCTCA | ATGCAACATCTAGAATATCCGTAC |
| NA | AGTAATGGCAACACTTACTTGTTT | (SEQ ID NO: 15) | ACG (SEQ ID NO: 17) |
| | (SEQ ID NO: 13) | | |
| AA | R S D K S L L H | R M S N L A S | M Q H L E Y P Y |
| | S N G N T Y L F | (SEQ ID NO: 16) | T (SEQ ID NO: 18) |
| | (SEQ ID NO: 14) | | |
| A-4 | AAGGCAAGTGAGGACATATATATT | GCTGCAACCAGTTTGGAACT | CAACAATATTGGACTACTCCGTGG |
| NA | CGCTTAGCC | (SEQ ID NO: 51) | ACG |
| | (SEQ ID NO: 49) | | (SEQ ID NO: 53) |
| AA | K A S E D I Y I | A A T S L E T | Q Q Y W T T P W |
| | R L A (SEQ ID NO: 50) | (SEQ ID NO: 52) | T (SEQ ID NO: 54) |
| A-5 | AGGTCTAGTAAGAGTCTCCTGCAT | CGGATGTCCAACCTTGCCTCA | ATGCAACATCTAGAATATCCGTAC |
| NA | AGTAATGGCAACACTTACTTGTAT | (SEQ ID NO: 57) | ACG |
| | (SEQ ID NO: 55) | | (SEQ ID NO: 59) |
| AA | R S S K S L L H | R M S N L A S | M Q H L E Y P Y |
| | S N G N T Y L Y | (SEQ ID NO: 58) | T |
| | (SEQ ID NO: 56) | | (SEQ ID NO: 60) |
| A-6 | AGGTCTAGTAAGAGTCTCCTGCAT | CGGATGTCCAACCTTGCCTCA | ATGCAACATCTAGAATATCCGTAC |
| | AGTAATGGCAACACTTACTTGTTT | (SEQ ID NO: 63) | ACG |
| | (SEQ ID NO: 61) | | (SEQ ID NO: 65) |
| AA | R S S K S L L H | R M S N L A S | M Q H L E Y P Y T |
| | S N G N T Y L F | (SEQ ID NO: 64) | (SEQ ID NO: 66) |
| | (SEQ ID NO: 62) | | |

HEAVY CHAINS H1 TO H3

| mAb | CDR 1 | CDR 2 | CDR 3 |
|---|---|---|---|
| A-1 | GATTATACCTGGAAC | TACATAACTTACAGTGGTAGCACT | CTGGGACGTCGCTATACTATGGAC |
| NA | (SEQ ID NO: 19) | AGCTACAACCCATCTCTCAAAAGT | TAC (SEQ ID NO: 23) |
| | | (SEQ ID NO: 21) | |
| AA | D Y T W N | Y I T Y S G S T S | L G R R Y T M D |
| | (SEQ ID NO: 20) | Y N P S L K S | Y (SEQ ID NO: 24) |
| | | (SEQ ID NO: 22) | |
| A-2 | ACCTACGCCATGAAC | CACATAAGAAGTAAAAGTAATAAT | CAAGGGGGTGACTTTCCTATGGAC |
| NA | (SEQ ID NO: 25) | TTTGCAACATATTATGCCGATTCA | TAC (SEQ ID NO: 29) |
| | | GTGAAAGAC (SEQ ID NO: 27) | |
| AA | T Y A M N | H I R S K S N N | Q G G D F P M D |
| | (SEQ ID NO: 26) | F A T Y Y A D S | Y (SEQ ID NO: 30) |
| | | V K D (SEQ ID NO: 28) | |
| A-3 | AGTTCCTGGTTGAAC | CGGATTTATCCTGGAGATGGAGAA | TATTATGAAGGGGGTTAT |
| NA | (SEQ ID NO: 31) | AATCACTATAATGGGAAATTCAAG | (SEQ ID NO: 35) |
| | | GGC (SEQ ID NO: 33) | |
| AA | S S W L N | R I Y P G D G E | Y Y E G G Y |
| | (SEQ ID NO: 32) | N H Y N G K F K | (SEQ ID NO: 36) |
| | | G (SEQ ID NO: 34) | |
| A-4 | ATTGATTATACCTGGAAC | TATATAACGTACAGTGGTAGCACT | CTGGGACGTCGCTATGCTTTGGAC |
| NA | (SEQ ID NO: 67) | GACTACAACCCATCTCTCAAAAGT | TAT |
| | | (SEQ ID NO: 69) | (SEQ ID NO: 71) |
| AA | I D Y T W N | Y I T Y S G S T | L G R R Y A L D Y |
| | (SEQ ID NO: 68) | D Y N P S L K S | (SEQ ID NO: 72) |
| | | (SEQ ID NO: 70) | |
| A-5 | AACTCCTGGATGAAC | CGGATTTATCCTGGAGATGGAGAG | TACTATGAAGGGGGTTAC |
| | (SEQ ID NO: 73) | ACTAGCTACAATGGGGAGTTCGTG | (SEQ ID NO: 77) |
| | | GGC | |
| | | (SEQ ID NO: 75) | |
| AA | N S W M N | R I Y P G D G E | Y Y E G G Y |
| | (SEQ ID NO: 74) | T S Y N G E F V | (SEQ ID NO: 78) |
| | | G (SEQ ID NO: 76) | |
| A-6 | AGCTCCTGGATGAAC | CGGATTTATCCTGGAGATGGAGAG | TACTATGAAGGGGGTTAC |
| | (SEQ ID NO: 79) | ACTAGCTACAATGGGGAGTTCAAG | (SEQ ID NO: 83) |
| | | GGC (SEQ ID NO: 81) | |
| AA | S S W M N | R I Y P G D G E | Y Y E G G Y |
| | (SEQ ID NO: 80) | T S Y N G E F K | (SEQ ID NO: 84) |
| | | G (SEQ ID NO: 82) | |

In another aspect, the present invention provides antigen binding proteins that comprise a light chain variable region selected from the group consisting of L1-L3 or a heavy chain variable region selected from the group consisting of H1-H3, and fragments, derivatives, muteins, and variants thereof. Such an antigen binding protein can be denoted using the nomenclature “LxHy”, wherein “x” corresponds to the number of the light chain variable region and “y” corresponds to the number of the heavy chain variable region. For example, L2H1 refers to an antigen binding protein with a light chain variable region comprising the amino acid sequence of L2 and a heavy chain variable region comprising the amino acid sequence of H1 as shown in Table 3 below. The CDR and framework regions of each of these variable domain sequences are also identified in Table 3 below. Antigen binding proteins of the invention include, for example, antibodies having a combination of light chain and heavy chain variable domains selected from the group of combinations consisting of L1H1, L2H2, L3H3, L4H4, L5H5, and L6H6. In one embodiment, the antibodies are human antibodies. In another embodiment, the antibodies are humanized antibodies.

Table 3 below also provides the polynucleotide (DNA) sequences encoding the amino acid sequences of the variable light and variable heavy domains for exemplary anti-c-MPL antibodies.

TABLE 3
Anti-c-MPL Variable Region Polynucleotide Sequences and Amino Acid Sequences
Ab 1.6.1 light chain (SEQ ID NOs: 37 & 38):

| | D I Q M T Q S S S S F S V S L G D R V T |
|---|---|
| 1 | GACATCCAGATGACACAATCTTCATCCTCCTTTTCTGTATCTCTAGGAGACAGAGTCACC |
| | I T C M A S E D I Y I R L A W Y Q Q K P |
| 61 | ATTACTTGCATGGCAAGTGAGGACATTTATATTCGCTTAGCCTGGTATCAGCAGAAACCA |
| | G N A P R L L I S A A T S L E T G V P S |
| 121 | GGAAATGCTCCTAGGCTCTTAATATCTGCTGCAACCAGTTTGGAAACTGGGGTTCCTTCT |
| | R F S G S G S G K D Y T L S I T S L Q T |
| 181 | AGATTCAGTGGCAGTGGATCTGGAAAGGATTACACTCTCAGCATTACCAGTCTACAGACT |
| | E D V G T Y Y C Q Q Y W S S P W T F G G |
| 241 | GAAGATGTTGGTACATATTACTGTCAACAGTATTGGAGTTCTCCGTGGACGTTCGGTGGA |
| | G T K L E I N |
| 301 | GGCACCAAGCTGGAAATCAAT |

Ab 1.6.1 heavy chain (SEQ ID NOs: 39 & 40):

| | |
|---|---|
| | D V Q L Q E S G P G L V K P S Q S L S L |
| 1 | GATGTGCAGCTTCAGGAGTCGGGACCTGGCCTGGTGAAACCTTCTCAGTCTCTGTCCCTC |
| | T C T V T G Y S I T S D Y T W N W I R Q |
| 61 | ACCTGCACTGTCACTGGCTACTCAATCACCAGTGATTATACCTGGAACTGGATCCGGCAG |
| | F P G N K L E W M G Y I T Y S G S T S Y |
| 121 | TTTCCAGGAAACAAACTGGAGTGGATGGGCTACATAACTTACAGTGGTAGCACTAGCTAC |
| | N P S L K S R N S I T R D T S K N Q F F |
| 181 | AACCCATCTCTCAAAAGTCGAAACTCTATCACTCGAGACACATCCAAGAACCAGTTCTTC |
| | L Q L N S V T T E D T A T Y Y C A R L G |
| 241 | CTGCAGTTGAATTCTGTGACTACTGAGGACACAGCCACATATTACTGTGCAAGACTGGGA |
| | R R Y T M D Y W G Q G T S V T V S S |
| 301 | CGTCGCTATACTATGGACTACTGGGGTCAAGGAACCTCAGTCACCGTCTCCTCA |

Ab 1.75.1 light chain (SEQ ID NOs: 41 & 42):

| | |
|---|---|
| | Q I V L T Q S P A I M S A S P G E K V T |
| 1 | CAAATTGTTCTCACCCAGTCTCCAGCAATCATGTCTGCATCTCCAGGGGAGAAGGTCACC |
| | I S C S A S S S V S Y M Y W Y Q Q K P G |
| 61 | ATATCCTGCAGTGCCAGCTCAAGTGTAAGTTACATGTACTGGTACCAGCAGAAGCCAGGA |
| | S S P K P W I Y R T S N L A S G V P A R |
| 121 | TCCTCCCCCAAACCCTGGATTTATCGCACATCCAACCTGGCTTCTGGAGTCCCTGCTCGC |
| | F S G S G S G T S Y S L T I S N M E A E |
| 181 | TTCAGTGGCAGTGGGTCTGGGACCTCTTACTCTCTCACAATCAGCAACATGGAGGCTGAA |
| | D A A A Y Y C Q Q Y H S Y P T T F G G G |
| 241 | GATGCTGCCGCTTATTACTGCCAGCAGTATCATAGTTACCCAACCACGTTCGGAGGGGGG |
| | T K L E V K |
| 301 | ACCAAGCTGGAAGTGAAA |

Ab 1.75.1 heavy chain (SEQ ID NOs: 43 & 44):

| | |
|---|---|
| | E V Q L V E S G G G L V Q P K G S L K L |
| 1 | GAGGTGCAGCTTGTTGAGTCTGGTGGAGGATTGGTGCAGCCTAAAGGGTCATTGAAACTC |
| | S C A A S G F S F N T Y A M N W V R Q A |
| 61 | TCATGTGCAGCCTCTGGATTCAGCTTCAATACCTACGCCATGAACTGGGTCCGCCAGGCT |
| | P G K G L E W I A H I R S K S N N F A T |
| 121 | CCAGGAAAGGGTTTGGAATGGATTGCTCACATAAGAAGTAAAAGTAATAATTTTGCAACA |
| | Y Y A D S V K D R F S I S R D A S E N I |
| 181 | TATTATGCCGATTCAGTGAAAGACAGATTCAGCATCTCCAGAGATGCTTCAGAAAACATT |
| | L F L Q M N N L K T E D T A M Y Y C V R |
| 241 | CTCTTTCTGCAAATGAACAACTTGAAAACTGAGGACACAGCCATGTATTATTGTGTGAGA |
| | Q G G D F P M D Y W G Q G T S V T V S S |
| 301 | CAAGGGGGTGACTTTCCTATGGACTACTGGGGTCAAGGAACCTCAGTCACCGTCTCCTCA |

Ab 1.78.1 light chain (SEQ ID NOs: 45 & 46):

| | |
|---|---|
| | D I V M T Q A A P S I P V T P G E S V S |
| 1 | GATATTGTGATGACTCAGGCTGCACCCTCTATTCCTGTCACTCCTGGAGAGTCAGTATCC |
| | I S C R S D K S L L H S N G N T Y L F W |
| 61 | ATCTCCTGCAGGTCTGATAAGAGTCTCCTGCATAGTAATGGCAACACTTACTTGTTTTGG |
| | F L Q R P G Q S P Q L L I Y R M S N L A |
| 121 | TTCCTGCAGAGGCCAGGCCAGTCTCCTCAGCTCCTGATATATCGGATGTCCAACCTTGCC |
| | S G V P D R F S G S G S G T A F T L R I |
| 181 | TCAGGAGTCCCAGACAGGTTCAGTGGCAGTGGGTCAGGAACTGCTTTCACACTGAGAATC |
| | S G V E A E D V G V Y Y C M Q H L E Y P |
| 241 | AGTGGAGTGGAGGCTGAGGATGTGGGTGTTTATTACTGTATGCAACATCTAGAATATCCG |
| | Y T F G G G T K L E I K |
| 301 | TACACGTTCGGAGGGGGGACCAAGCTGGAAATAAAA |

Ab 1.78.1 heavy chain (SEQ ID NOs: 47 & 48):

| | |
|---|---|
| | Q V Q L Q Q S G P E L V K P G A S V K M |
| 1 | CAGGTTCAACTGCAGCAGTCTGGACCTGAACTGGTGAAGCCTGGGGCCTCAGTGAAGATG |
| | S C K A S G Y A F S S S W L N W V R Q R |
| 61 | TCCTGCAAGGCTTCTGGCTACGCATTCAGTAGTTCCTGGTTGAACTGGGTGAGGCAGAGG |
| | P G K G L E W I G R I Y P G D G E N H Y |
| 121 | CCTGGAAAGGGTCTTGAGTGGATTGGACGGATTTATCCTGGAGATGGAGAAAATCACTAT |
| | N G K F K G K A T L T A D K S S S T G Y |
| 181 | AATGGGAAATTCAAGGGCAAGGCCACACTGACTGCAGACAAATCCTCCAGCACAGGCTAC |
| | M Q L S S L T S E D S A V Y F C A S Y Y |
| 241 | ATGCAACTCAGCAGCCTGACGTCTGAGGACTCTGCGGTCTACTTCTGTGCAAGTTATTAT |
| | E G G Y W G Q G T L I T V S A |
| 301 | GAAGGGGGTTATTGGGGCCAAGGGACTCTAATCACTGTCTCTGCA |

Ab 1.111.1 light chain (SEQ ID NOs: 85 & 86):

| | |
|---|---|
| | D I Q M T Q S S S S F S V S L G D R V T |
| 1 | GACATCCAGATGACACAATCTTCATCCTCCTTTTCTGTGTCTCTAGGAGACAGAGTCACC |
| | I T C K A S E D I Y I R L A W Y Q Q K P |
| 61 | ATTACTTGCAAGGCAAGTGAGGACATATATATTCGCTTAGCCTGGTATCAGCAGAAACCA |
| | G N A P R L L I S A A T S L E T G I P S |
| 121 | GGAAATGCTCCTAGGCTCTTAATATCTGCTGCAACCAGTTTGGAAACTGGGATTCCTTCA |
| | R F S G S G S G E D Y T L T I T S L Q T |
| 181 | AGATTCAGTGGCAGTGGATCTGGAGAGGATTACACTCTCACCATTACCAGTCTTCAGACT |
| | E D V A T Y Y C Q Q Y W T T P W T F G G |
| 241 | GAAGATGTTGCTACTTATTACTGTCAACAATATTGGACTACTCCGTGGACGTTCGGTGGA |
| | G T K L E I K R |
| 301 | GGCACCAAGCTGGAAATCAAACGG |

Ab 1.111.1 heavy chain (SEQ ID NOs: 87 & 88):

| | |
|---|---|
| | D V Q L Q E S G P G L V K P S Q S L S L |
| 1 | GATGTGCAACTTCAGGAGTCGGGACCTGGCCTGGTGAAACCTTCTCAGTCTCTGTCCCTC |
| | T C T V T G Y S I T I D Y T W N W I R Q |
| 61 | ACCTGCACTGTCACTGGCTACTCAATCACCATTGATTATACCTGGAACTGGATCCGGCAG |
| | F P G N K L E W M G Y I T Y S G S T D Y |
| 121 | TTTCCAGGAAACAAACTGGAGTGGATGGGCTATATAACGTACAGTGGTAGCACTGACTAC |
| | N P S L K S R S S I T R D T S M N Q F F |
| 181 | AACCCATCTCTCAAAAGTCGAAGCTCTATCACTCGAGACACATCCATGAACCAGTTCTTC |
| | L Q L N S V T T E D T A T Y Y C A R L G |
| 241 | CTGCAATTGAATTCTGTGACTACTGAGGACACAGCCACATATTACTGTGCAAGACTGGGA |
| | R R Y A L D Y W G Q G T S V T V S S |
| 301 | CGTCGCTATGCTTTGGACTATTGGGGTCAAGGAACCTCAGTCACCGTCTCCTCA |

Ab 1.36.1 light chain (SEQ ID NOs: 89 & 90):

| | |
|---|---|
| | D I V M T Q A A P S V P V T P G E S V S |
| 1 | GATATTGTGATGACTCAGGCTGCACCCTCTGTACCTGTCACTCCTGGAGAGTCAGTATCC |
| | I S C R S S K S L L H S N G N T Y L Y W |
| 61 | ATCTCCTGCAGGTCTAGTAAGAGTCTCCTGCATAGTAATGGCAACACTTACTTGTATTGG |
| | F L Q R P G Q S P Q L L I Y R M S N L A |
| 121 | TTCCTGCAGAGGCCAGGCCAGTCTCCTCAACTCCTGATATATCGGATGTCCAACCTTGCC |
| | S G V P D R F S G S G S G T A F T L R I |
| 181 | TCAGGAGTCCCAGACAGGTTCAGTGGCAGTGGGTCAGGAACTGCTTTCACACTGAGAATC |
| | S R V E A E D V G V Y Y C M Q H L E Y P |
| 241 | AGTAGAGTGGAGGCTGAGGATGTGGGTGTTTATTACTGTATGCAACATCTAGAATATCCG |
| | Y T F G G G T K L E I K R |
| 301 | TACACGTTCGGAGGGGGGACCAAGCTGGAAATAAAACGG |

Ab 1.36.1 heavy chain (SEQ ID NOs: 91 & 92):

| | |
|---|---|
| | Q V Q L Q Q S G P E L V K P G A S V K I |
| 1 | CAGGTTCAGCTACAGCAGTCTGGACCTGAGCTGGTGAAGCCTGGGGCCTCAGTGAAGATT |
| | S C K A S G Y G F S N S W M N W V R Q R |
| 61 | TCCTGCAAGGCTTCTGGCTACGGATTCAGTAACTCCTGGATGAACTGGGTGAGGCAGAGG |
| | P G K G L E W I G R I Y P G D G E T S Y |
| 121 | CCTGGAAAGGGTCTTGAGTGGATTGGACGGATTTATCCTGGAGATGGAGAGACTAGCTAC |
| | N G E F V G K A T L T A D K S S S T A Y |
| 181 | AATGGGGAGTTCGTGGGCAAGGCCACACTGACTGCAGACAAATCTTCCAGCACAGCCTAC |
| | M H L S S L T S E D S A V Y F C A S Y Y |
| 241 | ATGCACCTCAGCAGCCTGACATCTGAGGACTCTGCGGTCTACTTCTGTGCAAGCTACTAT |
| | E G G Y W G Q G T L V T V S |
| 301 | GAAGGGGGTTACTGGGGCCAAGGGACTCTGGTCACTGTCTCT |

Ab 1.169.1 light chain (SEQ ID NOs: 93 & 94):

| | |
|---|---|
| | D I V M T Q A A P S L P V T P G E S V S |
| 1 | GATATTGTGATGACTCAGGCTGCACCCTCTCTTCCTGTCACTCCTGGAGAGTCAGTATCC |
| | I S C R S S K S L L H S N G N T Y L F W |
| 61 | ATCTCCTGCAGGTCTAGTAAGAGTCTCCTGCATAGTAATGGCAACACTTACTTGTTTTGG |
| | F L Q R P G Q S P H L L I Y R M S N L A |
| 121 | TTCCTGCAGAGGCCAGGCCAGTCTCCTCACCTCCTGATATATCGGATGTCCAACCTTGCC |
| | S G V P D R F S G S G S G T A F T L R I |
| 181 | TCAGGAGTCCCAGACAGGTTCAGTGGCAGTGGGTCAGGAACTGCTTTCACACTGAGAATC |
| | S R V E A E D V G V Y Y C M Q H L E Y P |
| 241 | AGTAGAGTGGAGGCTGAGGATGTGGGTGTTTATTACTGTATGCAACATCTAGAATATCCG |
| | Y T F G G G T K L E I K R |
| 301 | TACACGTTCGGAGGGGGGACCAAGCTGGAAATAAAACGG |

Ab 1.169.1 heavy chain (SEQ ID NOs: 95 & 96):

| | |
|---|---|
| | Q V Q L Q Q S G P E L V K P G A S V K I |
| 1 | CAGGTTCAGCTGCAGCAGTCTGGACCTGAGCTGGTGAAGCCTGGGGCCTCAGTGAAGATT |
| | S C K A S G Y G F S S S W M N W V K Q R |
| 61 | TCCTGCAAGGCTTCTGGCTACGGATTCAGTAGCTCCTGGATGAACTGGGTGAAGCAGAGG |
| | P G K G L E W I G R I Y P G D G E T S Y |
| 121 | CCTGGAAAGGGTCTTGAGTGGATTGGACGGATTTATCCTGGAGATGGAGAGACTAGCTAC |
| | N G E F K G K A T L T A D K S S S T A Y |
| 181 | AATGGGGAGTTCAAGGGCAAGGCCACACTGACTGCCGACAAATCCTCCAGCACAGCCTAC |
| | M Q L S S L T S E D S A V Y F C A S Y Y |
| 241 | ATGCAACTCAGCAGCCTGACATCTGAGGACTCTGCGGTCTACTTCTGCGCAAGCTACTAT |
| | E G G Y W G Q G T L V T V S A |
| 301 | GAAGGGGGTTACTGGGGCCAAGGGACTCTGGTCACTGTCTCTGCA |

Particular embodiments of antigen binding proteins of the present invention comprise one or more amino acid sequences that are identical to the amino acid sequences of one or more of the CDRs and/or FRs (framework regions) illustrated above. In one embodiment, the antigen binding protein comprises a light chain CDR1 sequence illustrated above. In another embodiment, the antigen binding protein comprises a light chain CDR2 sequence illustrated above. In another embodiment, the antigen binding protein comprises a light chain CDR3 sequence illustrated in above. In another embodiment, the antigen binding protein comprises a heavy chain CDR1 sequence illustrated in above. In another embodiment, the antigen binding protein comprises a heavy chain CDR2 sequence illustrated above. In another embodiment, the antigen binding protein comprises a heavy chain CDR3 sequence illustrated above. In another embodiment, the antigen binding protein comprises a light chain FR1 sequence illustrated above. In another embodiment, the antigen binding protein comprises a light chain FR2 sequence illustrated above. In another embodiment, the antigen binding protein comprises a light chain FR3 sequence illustrated above. In another embodiment, the antigen binding protein comprises a light chain FR4 sequence illustrated above. In another embodiment, the antigen binding protein comprises a heavy chain FR1 sequence illustrated above. In another embodiment, the antigen binding protein comprises a heavy chain FR2 sequence illustrated above. In another embodiment, the antigen binding protein comprises a heavy chain FR3 sequence illustrated above. In another embodiment, the antigen binding protein comprises a heavy chain FR4 sequence illustrated above.

In another embodiment, at least one of the antigen binding protein's CDR3 sequences differs by no more than 6, 5, 4, 3, 2, 1 or 0 single amino acid addition, substitution, and/or deletion from a CDR3 sequence from A1-A6, as shown in Tables 2 and 3 above. In another embodiment, the antigen binding protein's light chain CDR3 sequence differs by no more than 6, 5, 4, 3, 2, 1 or 0 single amino acid addition, substitution, and/or deletion from a light chain CDR3 sequence from A1-A6 as shown above and the antigen binding protein's heavy chain CDR3 sequence differs by no more than 6, 5, 4, 3, 2, 1 or 0 single amino acid addition, substitution, and/or deletion from a heavy chain CDR3 sequence from A1-A6 as shown above. In another embodiment, the antigen binding protein further comprises 1, 2, 3, 4, or 5 CDR sequences that each independently differs by 6, 5, 4, 3, 2, 1, or 0 single amino acid additions, substitutions, and/or deletions from a CDR sequence of A1-A6. In another embodiment, the antigen binding protein comprises the CDRs of the light chain variable region and the CDRs of the heavy chain variable region set forth above. In another embodiment, the antigen binding protein comprises 1, 2, 3, 4, 5, and/or 6 consensus CDR sequences shown above. In a further embodiment, the antigen binding protein comprises the CDRs of any one of L1H1, L2H2, L3H3, L4H4, L5H5, or L6H6. In one embodiment, the antigen binding protein is a human antibody. In another embodiment, the antigen binding protein is a humanized antibody.

In one embodiment, the antigen binding protein (such as an antibody or antibody fragment) comprises a light chain variable domain comprising a sequence of amino acids that differs from the sequence of a light chain variable domain selected from the group consisting of L1 through L6 only at 15, 14, 13, 12, 11, 10, 9, 8, 7, 6, 5, 4, 3, 2, 1 or 0 residues, wherein each such sequence difference is independently either a deletion, insertion, or substitution of one amino acid residue. In another embodiment, the light-chain variable domain comprises a sequence of amino acids that is at least 70%, 75%, 80%, 85%, 90%, 95%, 97%, or 99% identical to the sequence of a light chain variable domain selected from the group consisting of L1-L6. In another embodiment, the light chain variable domain comprises a sequence of amino acids that is encoded by a nucleotide sequence that is at least 70%, 75%, 80%, 85%, 90%, 95%, 97%, or 99% identical to a L1-L6 polynucleotide sequence listed below. In another embodiment, the light chain variable domain comprises a sequence of amino acids that is encoded by a polynucleotide that hybridizes under moderately stringent conditions to the complement of a polynucleotide that encodes a light chain variable domain selected from the group consisting of L1-L6. In another embodiment, the light chain variable domain comprises a sequence of amino acids that is encoded by a polynucleotide that hybridizes under stringent conditions to the complement of a polynucleotide that encodes a light chain variable domain selected from the group consisting of L1-L6.

In another embodiment, the present invention provides an antigen binding protein comprising a heavy chain variable domain comprising a sequence of amino acids that differs from the sequence of a heavy chain variable domain selected from the group consisting of H1-H6 only at 15, 14, 13, 12, 11, 10, 9, 8, 7, 6, 5, 4, 3, 2, 1 or 0 residue(s), wherein each such sequence difference is independently either a deletion, insertion, or substitution of one amino acid residue. In another embodiment, the heavy chain variable domain comprises a sequence of amino acids that is at least 70%, 75%, 80%, 85%, 90%, 95%, 97%, or 99% identical to the sequence of a heavy chain variable domain selected from the group consisting of H1-H6. In another embodiment, the heavy chain variable domain comprises a sequence of amino acids that is encoded by a nucleotide sequence that is at least 70%, 75%, 80%, 85%, 90%, 95%, 97%, or 99% identical to a nucleotide sequence that encodes a heavy chain variable domain selected from the group consisting of H1-H6. In another embodiment, the heavy chain variable domain comprises a sequence of amino acids that is encoded by a polynucleotide that hybridizes under moderately stringent conditions to the complement of a polynucleotide that encodes a heavy chain variable domain selected from the group consisting of H1-H6. In another embodiment, the heavy chain variable domain comprises a sequence of amino acids that is encoded by a polynucleotide that hybridizes under stringent conditions to the complement of a polynucleotide that encodes a heavy chain variable domain selected from the group consisting of H1-H6.

Additional embodiments include antigen binding proteins comprising the combinations L1H1, L2H2, L3H3, L4H4, L5H5, or L6H6.

Antigen binding proteins (e.g., antibodies, antibody fragments, and antibody derivatives) of the invention can comprise any constant region known in the art. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. In one embodiment, the light or heavy chain constant region is a fragment, derivative, variant, or mutein of a naturally occurring constant region.

Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See also Lanitto et al., Methods Mol. Biol. 178:303-16 (2002).

The antigen binding proteins (for example, antibodies) of the present invention include those comprising, for example, the variable domain combinations L1H1, L2H2, L3H3, L4H4, L5H5, and L6H6 having a desired isotype (for example, IgA, IgG1, IgG2, IgG3, IgG4, IgM, IgE, and IgD) as well as Fab or F(ab′)₂ fragments thereof. Moreover, if an IgG4 is desired, it may also be desired to introduce a point mutation in the hinge region as described in Bloom et al., 1997, Protein Science 6:407, (incorporated by reference herein) to alleviate a tendency to form intra-H chain disulfide bonds that can lead to heterogeneity in the IgG4 antibodies.

### Antibodies and Antibody Fragments

In one embodiment the antigen binding proteins are antibodies. The term “antibody” refers to an intact antibody, or an antigen binding fragment thereof, as described extensively in the definition section. An antibody may comprise a complete antibody molecule (including polyclonal, monoclonal, chimeric, humanized, or human versions having full length heavy and/or light chains), or comprise an antigen binding fragment thereof. Antibody fragments include F(ab′)₂, Fab, Fab′, Fv, Fc, and Fd fragments, and can be incorporated into single domain antibodies, single-chain antibodies, maxibodies, minibodies, intrabodies, diabodies, triabodies, tetrabodies, v-NAR and bis-scFv (see e.g., Hollinger and Hudson, 2005, Nature Biotechnology, 23, 9, 1126-1136). Also included are antibody polypeptides such as those disclosed in U.S. Pat. No. 6,703,199, including fibronectin polypeptide monobodies. Other antibody polypeptides are disclosed in U.S. Patent Publication 2005/0238646, which are single-chain polypeptides. In one embodiment, the antibodies of the present invention comprise at least one CDR set forth in Table 2 above. In another aspect, the present invention provides hybridomas capable of producing the antibodies of the invention, and methods of producing antibodies from hybridomas, as described further below.

Chimeric antibodies and humanized antibodies are defined in the definition section and may be prepared by known techniques. In one embodiment, a humanized monoclonal antibody comprises the variable domain of a murine antibody (or all or part of the antigen binding site thereof) and a constant domain derived from a human antibody. Alternatively, a humanized antibody fragment may comprise the antigen binding site of a murine monoclonal antibody and a variable domain fragment (lacking the antigen-binding site) derived from a human antibody. Procedures for the production of engineered monoclonal antibodies include those described in Riechmann et al., 1988, Nature 332:323, Liu et al., 1987, Proc. Nat. Acad. Sci. USA 84:3439, Larrick et al., 1989, Bio/Technology 7:934, and Winter et al., 1993, TIPS 14:139. In one embodiment, the chimeric antibody is a CDR grafted antibody. Techniques for humanizing antibodies are discussed in, e.g., U.S. Pat. Nos. 5,869,619; 5,225,539; 5,821,337; 5,859,205; 6,881,557, Padlan et al., 1995, FASEB J. 9:133-39, Tamura et al., 2000, J. Immunol. 164:1432-41, Zhang, W., et al., Molecular Immunology. 42(12):1445-1451, 2005; Hwang W. et al., Methods. 36(1):35-42, 2005; Dall'Acqua W F, et al., Methods 36(1):43-60, 2005; and Clark, M., Immunology Today. 21(8):397-402, 2000.

An antibody of the present invention may also be a fully human monoclonal antibody. Fully human monoclonal antibodies may be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B-cells, fusion of spleen cells from immunized transgenic mice carrying inserted human immunoglobulin genes, isolation from human immunoglobulin V region phage libraries, or other procedures as known in the art and based on the disclosure herein.

Procedures have been developed for generating human monoclonal antibodies in non-human animals. For example, mice in which one or more endogenous immunoglobulin genes have been inactivated by various means have been prepared. Human immunoglobulin genes have been introduced into the mice to replace the inactivated mouse genes. In this technique, elements of the human heavy and light chain locus are introduced into strains of mice derived from embryonic stem cell lines that contain targeted disruptions of the endogenous heavy chain and light chain loci (see also Bruggemann et al., Curr. Opin. Biotechnol. 8:455-58 (1997)). For example, human immunoglobulin transgenes may be mini-gene constructs, or transloci on yeast artificial chromosomes, which undergo B-cell-specific DNA rearrangement and hypermutation in the mouse lymphoid tissue.

Antibodies produced in the animal incorporate human immunoglobulin polypeptide chains encoded by the human genetic material introduced into the animal. In one embodiment, a non-human animal, such as a transgenic mouse, is immunized with a suitable c-MPL immunogen. One example of a suitable c-MPL immunogen is the extracellular domain of SEQ ID NO: 99.

Examples of techniques for production and use of transgenic animals for the production of human or partially human antibodies are described in U.S. Pat. Nos. 5,814,318, 5,569,825, and 5,545,806, Davis et al., Production of human antibodies from transgenic mice in Lo, ed. Antibody Engineering: Methods and Protocols, Humana Press, NJ:191-200 (2003), Kellermann et al., 2002, Curr Opin Biotechnol. 13:593-97, Russel et al., 2000, Infect Immun. 68:1820-26, Gallo et al., 2000, Eur J. Immun. 30:534-40, Davis et al., 1999, Cancer Metastasis Rev. 18:421-25, Green, 1999, J Immunol Methods. 231:11-23, Jakobovits, 1998, Advanced Drug Delivery Reviews 31:33-42, Green et al., 1998, J Exp Med. 188:483-95, Jakobovits A, 1998, Exp. Opin. Invest. Drugs. 7:607-14, Tsuda et al., 1997, Genomics. 42:413-21, Mendez et al., 1997, Nat. Genet. 15:146-56, Jakobovits, 1994, Curr Biol. 4:761-63, Arbones et al., 1994, Immunity. 1:247-60, Green et al., 1994, Nat. Genet. 7:13-21, Jakobovits et al., 1993, Nature. 362:255-58, Jakobovits et al., 1993, Proc Natl Acad Sci USA. 90:2551-55. Chen, J., M. Trounstine, F. W. Alt, F. Young, C. Kurahara, J. Loring, D. Huszar. “Immunoglobulin gene rearrangement in B-cell deficient mice generated by targeted deletion of the JH locus.” International Immunology 5 (1993): 647-656, Choi et al., 1993, Nature Genetics 4: 117-23, Fishwild et al., 1996, Nature Biotechnology 14: 845-51, Harding et al., 1995, Annals of the New York Academy of Sciences, Lonberg et al., 1994, Nature 368: 856-59, Lonberg, 1994, Transgenic Approaches to Human Monoclonal Antibodies in Handbook of Experimental Pharmacology 113: 49-101, Lonberg et al., 1995, Internal Review of Immunology 13: 65-93, Neuberger, 1996, Nature Biotechnology 14: 826, Taylor et al., 1992, Nucleic Acids Research 20: 6287-95, Taylor et al., 1994, International Immunology 6: 579-91, Tomizuka et al., 1997, Nature Genetics 16: 133-43, Tomizuka et al., 2000, Proceedings of the National Academy of Sciences USA 97: 722-27, Tuaillon et al., 1993, Proceedings of the National Academy of Sciences USA 90: 3720-24, and Tuaillon et al., 1994, Journal of Immunology 152: 2912-20.; Lonberg et al., Nature 368:856, 1994; Taylor et al., Int. Immun. 6:579, 1994; U.S. Pat. No. 5,877,397; Bruggemann et al., 1997 Curr. Opin. Biotechnol. 8:455-58; Jakobovits et al., 1995 Ann. N.Y. Acad. Sci. 764:525-35. In addition, protocols involving the XenoMouse® (Abgenix, now Amgen, Inc.) are described, for example in U.S. 05/0118643 and WO 05/694879, WO 98/24838, WO 00/76310, and U.S. Pat. No. 7,064,244.

Lymphoid cells from the immunized transgenic mice are fused with myeloma cells for example to produce hybridomas. Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in such fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

The lymphoid (e.g., spleen) cells and the myeloma cells may be combined for a few minutes with a membrane fusion-promoting agent, such as polyethylene glycol or a nonionic detergent, and then plated at low density on a selective medium that supports the growth of hybridoma cells but not unfused myeloma cells. One selection media is HAT (hypoxanthine, aminopterin, thymidine). After a sufficient time, usually about one to two weeks, colonies of cells are observed. Single colonies are isolated, and antibodies produced by the cells may be tested for binding activity to human c-MPL using any one of a variety of immunoassays known in the art and described herein. The hybridomas are cloned (e.g., by limited dilution cloning or by soft agar plaque isolation) and positive clones that produce an antibody specific to human c-MPL are selected and cultured. The monoclonal antibodies from the hybridoma cultures may be isolated from the supernatants of hybridoma cultures. Thus the present invention provides hybridomas that comprise polynucleotides encoding the antigen binding proteins of the invention in the chromosomes of the cell. These hybridomas can be cultured according to methods described herein and known in the art.

Another method for generating human antibodies of the invention includes immortalizing human peripheral blood cells by EBV transformation. See, e.g., U.S. Pat. No. 4,464,456. Such an immortalized B-cell line (or lymphoblastoid cell line) producing a monoclonal antibody that specifically binds to human c-MPL can be identified by immunodetection methods as provided herein, for example, an ELISA, and then isolated by standard cloning techniques. The stability of the lymphoblastoid cell line producing an anti-c-MPL antibody may be improved by fusing the transformed cell line with a murine myeloma to produce a mouse-human hybrid cell line according to methods known in the art (see, e.g., Glasky et al., Hybridoma 8:377-89 (1989)). Still another method to generate human monoclonal antibodies is in vitro immunization, which includes priming human splenic B-cells with human c-MPL, followed by fusion of primed B-cells with a heterohybrid fusion partner. See, e.g., Boerner et al., 1991 J. Immunol. 147:86-95.

In certain embodiments, a B-cell that is producing an anti-human c-MPL antibody is selected and the light chain and heavy chain variable regions are cloned from the B-cell according to molecular biology techniques known in the art (WO 92/02551; U.S. Pat. No. 5,627,052; Babcook et al., Proc. Natl. Acad. Sci. USA 93:7843-48 (1996)) and described herein. B-cells from an immunized animal may be isolated from the spleen, lymph node, or peripheral blood sample by selecting a cell that is producing an antibody that specifically binds to c-MPL. B-cells may also be isolated from humans, for example, from a peripheral blood sample. Methods for detecting single B-cells that are producing an antibody with the desired specificity are well known in the art, for example, by plaque formation, fluorescence-activated cell sorting, in vitro stimulation followed by detection of specific antibody, and the like. Methods for selection of specific antibody-producing B-cells include, for example, preparing a single cell suspension of B-cells in soft agar that contains human c-MPL. Binding of the specific antibody produced by the B-cell to the antigen results in the formation of a complex, which may be visible as an immunoprecipitate. After the B-cells producing the desired antibody are selected, the specific antibody genes may be cloned by isolating and amplifying DNA or mRNA according to methods known in the art and described herein.

An additional method for obtaining antibodies of the invention is by phage display. See, e.g., Winter et al., 1994 Annu Rev. Immunol. 12:433-55; Burton et al., 1994 Adv. Immunol. 57:191-280. Human or murine immunoglobulin variable region gene combinatorial libraries may be created in phage vectors that can be screened to select Ig fragments (Fab, Fv, sFv, or multimers thereof) that bind specifically to TGF-beta binding protein or variant or fragment thereof. See, e.g., U.S. Pat. No. 5,223,409; Huse et al., 1989 Science 246:1275-81; Sastry et al., Proc. Natl. Acad. Sci. USA 86:5728-32 (1989); Alting-Mees et al., Strategies in Molecular Biology 3:1-9 (1990); Kang et al., 1991 Proc. Natl. Acad. Sci. USA 88:4363-66; Hoogenboom et al., 1992 J. Molec. Biol. 227:381-388; Schlebusch et al., 1997 Hybridoma 16:47-52 and references cited therein. For example, a library containing a plurality of polynucleotide sequences encoding Ig variable region fragments may be inserted into the genome of a filamentous bacteriophage, such as M13 or a variant thereof, in frame with the sequence encoding a phage coat protein. A fusion protein may be a fusion of the coat protein with the light chain variable region domain and/or with the heavy chain variable region domain. According to certain embodiments, immunoglobulin Fab fragments may also be displayed on a phage particle (see, e.g., U.S. Pat. No. 5,698,426).

Heavy and light chain immunoglobulin cDNA expression libraries may also be prepared in lambda phage, for example, using λlmmunoZap™(H) and λImmunoZap™(L) vectors (Stratagene, La Jolla, Calif.). Briefly, mRNA is isolated from a B-cell population, and used to create heavy and light chain immunoglobulin cDNA expression libraries in the λImmunoZap(H) and λImmunoZap(L) vectors. These vectors may be screened individually or co-expressed to form Fab fragments or antibodies (see Huse et al., supra; see also Sastry et al., supra). Positive plaques may subsequently be converted to a non-lytic plasmid that allows high level expression of monoclonal antibody fragments from E. coli.

In one embodiment, in a hybridoma the variable regions of a gene expressing a monoclonal antibody of interest are amplified using nucleotide primers. These primers may be synthesized by one of ordinary skill in the art, or may be purchased from commercially available sources. (See, e.g., Stratagene (La Jolla, Calif.), which sells primers for mouse and human variable regions including, among others, primers for V_{Ha}, V_{Hb}, V_{Hc}, V_{Hd}, C_{H1}, V_{L} and C_{L} regions.) These primers may be used to amplify heavy or light chain variable regions, which may then be inserted into vectors such as ImmunoZAP™H or ImmunoZAP™L (Stratagene), respectively. These vectors may then be introduced into E. coli, yeast, or mammalian-based systems for expression. Large amounts of a single-chain protein containing a fusion of the V_{H} and V_{L} domains may be produced using these methods (see Bird et al., Science 242:423-426, 1988).

Once cells producing antibodies according to the invention have been obtained using any of the above-described immunization and other techniques, the specific antibody genes may be cloned by isolating and amplifying DNA or mRNA therefrom according to standard procedures as described herein. The antibodies produced therefrom may be sequenced and the CDRs identified and the DNA coding for the CDRs may be manipulated as described previously to generate other antibodies according to the invention.

Antigen binding proteins of the present invention preferably modulate TPO-R signalling in the cell-based assay described herein and/or the in vivo assay described herein and/or cross-block the binding of one of the antibodies described in this application and/or are cross-blocked from binding TPO-R by one of the antibodies described in this application. Accordingly such binding agents can be identified using the assays described herein.

In certain embodiments, antibodies are generated by first identifying antibodies that bind to cells overexpressing TPO-R and/or neutralize in the cell-based and/or in vivo assays described herein and/or cross-block the antibodies described in this application and/or are cross-blocked from binding TPO-R by one of the antibodies described in this application.

It will be understood by one skilled in the art that some proteins, such as antibodies, may undergo a variety of posttranslational modifications. The type and extent of these modifications often depends on the host cell line used to express the protein as well as the culture conditions. Such modifications may include variations in glycosylation, methionine oxidation, diketopiperizine formation, aspartate isomerization and asparagine deamidation. A frequent modification is the loss of a carboxy-terminal basic residue (such as lysine or arginine) due to the action of carboxypeptidases (as described in Harris, R. J. Journal of Chromatography 705:129-134, 1995).

An alternative method for production of a murine monoclonal antibody is to inject the hybridoma cells into the peritoneal cavity of a syngeneic mouse, for example, a mouse that has been treated (e.g., pristane-primed) to promote formation of ascites fluid containing the monoclonal antibody. Monoclonal antibodies can be isolated and purified by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104 (The Humana Press, Inc. 1992)). Monoclonal antibodies may be purified by affinity chromatography using an appropriate ligand selected based on particular properties of the antibody (e.g., heavy or light chain isotype, binding specificity, etc.). Examples of a suitable ligand, immobilized on a solid support, include Protein A, Protein G, an anticonstant region (light chain or heavy chain) antibody, an anti-idiotype antibody, and a TGF-beta binding protein, or fragment or variant thereof.

Molecular evolution of the complementarity determining regions (CDRs) in the center of the antibody binding site also has been used to isolate antibodies with increased affinity, for example, antibodies having increased affinity for c-erbB-2, as described by Schier et al., 1996, J. Mol. Biol. 263:551. Accordingly, such techniques are useful in preparing antibodies to human MPL receptor.

Antigen binding proteins directed against human MPL receptor can be used, for example, in assays to detect the presence of the MPL receptor, either in vitro or in vivo.

Although human, partially human, or humanized antibodies will be suitable for many applications, particularly those involving administration of the antibody to a human subject, other types of antigen binding proteins will be suitable for certain applications. The non-human antibodies of the invention can be, for example, derived from any antibody-producing animal, such as mouse, rat, rabbit, goat, donkey, or non-human primate (for example, monkey such as cynomologus or rhesus monkey) or ape (e.g., chimpanzee)). Non-human antibodies of the invention can be used, for example, in in vitro and cell-culture based applications, or any other application where an immune response to the antibody of the invention does not occur, is insignificant, can be prevented, is not a concern, or is desired. In one embodiment, a non-human antibody of the invention is administered to a non-human subject. In another embodiment, the non-human antibody does not elicit an immune response in the non-human subject. In another embodiment, the non-human antibody is from the same species as the non-human subject, e.g., a mouse antibody of the invention is administered to a mouse. An antibody from a particular species can be made by, for example, immunizing an animal of that species with the desired immunogen or using an artificial system for generating antibodies of that species (e.g., a bacterial or phage display-based system for generating antibodies of a particular species), or by converting an antibody from one species into an antibody from another species by replacing, e.g., the constant region of the antibody with a constant region from the other species, or by replacing one or more amino acid residues of the antibody so that it more closely resembles the sequence of an antibody from the other species. In one embodiment, the antibody is a chimeric antibody comprising amino acid sequences derived from antibodies from two or more different species.

Antibodies also may be prepared by any of a number of conventional techniques. For example, they may be purified from cells that naturally express them (e.g., an antibody can be purified from a hybridoma that produces it), or produced in recombinant expression systems, using any technique known in the art. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988). This is discussed in the nucleic acid section below.

Where it is desired to improve the affinity of antibodies according to the invention containing one or more of the above-mentioned CDRs can be obtained by a number of affinity maturation protocols including maintaining the CDRs (Yang et al., J. Mol. Biol., 254, 392-403, 1995), chain shuffling (Marks et al., Bio/Technology, 10, 779-783, 1992), use of mutation strains of E. coli. (Low et al., J. Mol. Biol., 250, 350-368, 1996), DNA shuffling (Patten et al., Curr. Opin. Biotechnol., 8, 724-733, 1997), phage display (Thompson et al., J. Mol. Biol., 256, 7-88, 1996) and additional PCR techniques (Crameri, et al., Nature, 391, 288-291, 1998). All of these methods of affinity maturation are discussed by Vaughan et al. (Nature Biotechnology, 16, 535-539, 1998).

### Antibody Fragments

In another aspect, the present invention provides fragments of an anti-c-MPL receptor antibody of the invention. Such fragments can consist entirely of antibody-derived sequences or can comprise additional sequences. Examples of antigen-binding fragments include Fab, F(ab′)2, single chain antibodies, diabodies, triabodies, tetrabodies, and domain antibodies. Other examples are provided in Lunde et al., 2002, Biochem. Soc. Trans. 30:500-06.

Single chain antibodies may be formed by linking heavy and light chain variable domain (Fv region) fragments via an amino acid bridge (short peptide linker), resulting in a single polypeptide chain. Such single-chain Fvs (scFvs) have been prepared by fusing DNA encoding a peptide linker between DNAs encoding the two variable domain polypeptides (V_{L} and V_{H}). The resulting polypeptides can fold back on themselves to form antigen-binding monomers, or they can form multimers (e.g., dimers, trimers, or tetramers), depending on the length of a flexible linker between the two variable domains (Kortt et al., 1997, Prot. Eng. 10:423; Kortt et al., 2001, Biomol. Eng. 18:95-108). By combining different V_{L} and V_{H}-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol. Biol. 178:379-87. Single chain antibodies derived from antibodies provided herein include, but are not limited to, scFvs comprising the variable domain combinations L1H1, L2H2, L3H3, L4H4, L5H5, and L6H6 are encompassed by the present invention.

Antigen binding fragments derived from an antibody can also be obtained, for example, by proteolytic hydrolysis of the antibody, for example, pepsin or papain digestion of whole antibodies according to conventional methods. By way of example, antibody fragments can be produced by enzymatic cleavage of antibodies with pepsin to provide a 5S fragment termed F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using papain produces two monovalent Fab fragments and an Fc fragment directly. These methods are described, for example, by Goldenberg, U.S. Pat. No. 4,331,647, Nisonoff et al., Arch. Biochem. Biophys. 89:230, 1960; Porter, Biochem. J. 73:119, 1959; Edelman et al., in Methods in Enzymology 1:422 (Academic Press 1967); and by Andrews, S. M. and Titus, J. A. in Current Protocols in Immunology (Coligan J. E., et al., eds), John Wiley & Sons, New York (2003), pages 2.8.1-2.8.10 and 2.10A.1-2.10A.5. Other methods for cleaving antibodies, such as separating heavy chains to form monovalent light-heavy chain fragments (Fd), further cleaving of fragments, or other enzymatic, chemical, or genetic techniques may also be used, so long as the fragments bind to the antigen that is recognized by the intact antibody.

Another form of an antibody fragment is a peptide comprising one or more complementarity determining regions (CDRs) of an antibody. CDRs can be obtained by constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology 2:106, 1991; Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley-Liss, Inc. 1995)). The antibody fragment further may comprise at least one variable region domain of an antibody described herein. Thus, for example, the V region domain may be monomeric and be a V_{H} or V_{L} domain, which is capable of independently binding human c-MPL receptor with an affinity at least equal to 10⁻⁷ M or less as described below.

The variable region domain may be any naturally occurring variable domain or an engineered version thereof. By engineered version is meant a variable region domain that has been created using recombinant DNA engineering techniques. Such engineered versions include those created, for example, from a specific antibody variable region by insertions, deletions, or changes in or to the amino acid sequences of the specific antibody. Particular examples include engineered variable region domains containing at least one CDR and optionally one or more framework amino acids from a first antibody and the remainder of the variable region domain from a second antibody.

The variable region domain may be covalently attached at a C-terminal amino acid to at least one other antibody domain or a fragment thereof. Thus, for example, a VH domain that is present in the variable region domain may be linked to an immunoglobulin CH1 domain, or a fragment thereof. Similarly a V_{L} domain may be linked to a C_{K} domain or a fragment thereof. In this way, for example, the antibody may be a Fab fragment wherein the antigen binding domain contains associated V_{H} and V_{L} domains covalently linked at their C-termini to a CH1 and C_{K} domain, respectively. The CH1 domain may be extended with further amino acids, for example to provide a hinge region or a portion of a hinge region domain as found in a Fab′ fragment, or to provide further domains, such as antibody CH2 and CH3 domains.

### Derivatives and Variants of Antigen Binding Proteins

The nucleotide sequences of L1-L6 and H1-H6, encoding the corresponding amino acid sequences of A1-A6, can be altered, for example, by random mutagenesis or by site-directed mutagenesis (e.g., oligonucleotide-directed site-specific mutagenesis) to create an altered polynucleotide comprising one or more particular nucleotide substitutions, deletions, or insertions as compared to the non-mutated polynucleotide. Examples of techniques for making such alterations are described in Walder et al., 1986, Gene 42:133; Bauer et al. 1985, Gene 37:73; Craik, BioTechniques, January 1985, 12-19; Smith et al., 1981, Genetic Engineering: Principles and Methods, Plenum Press; and U.S. Pat. Nos. 4,518,584 and 4,737,462. These and other methods can be used to make, for example, derivatives of anti-c-MPL receptor antibodies that have a desired property, for example, increased affinity, avidity, or specificity for c-MPL receptor increased activity or stability in vivo or in vitro, or reduced in vivo side-effects as compared to the underivatized antibody.

Other derivatives of anti-c-MPL receptor antibodies within the scope of this invention include covalent or aggregative conjugates of anti-c-MPL receptor antibodies, or fragments thereof, with other proteins or polypeptides, such as by expression of recombinant fusion proteins comprising heterologous polypeptides fused to the N-terminus or C-terminus of an anti-c-MPL receptor antibody polypeptide. For example, the conjugated peptide may be a heterologous signal (or leader) polypeptide, e.g., the yeast alpha-factor leader, or a peptide such as an epitope tag. Antigen binding protein-containing fusion proteins can comprise peptides added to facilitate purification or identification of antigen binding protein (e.g., poly-His). An antigen binding protein also can be linked to the FLAG peptide as described in Hopp et al., Bio/Technology 6:1204, 1988, and U.S. Pat. No. 5,011,912. The FLAG peptide is highly antigenic and provides an epitope reversibly bound by a specific monoclonal antibody (mAb), enabling rapid assay and facile purification of expressed recombinant protein. Reagents useful for preparing fusion proteins in which the FLAG peptide is fused to a given polypeptide are commercially available (Sigma, St. Louis, Mo.).

In another embodiment, oligomers that contain one or more antigen binding proteins may be employed as c-MPL receptor antagonists. Oligomers may be in the form of covalently-linked or non-covalently-linked dimers, trimers, or higher oligomers. Oligomers comprising two or more antigen binding protein are contemplated for use, with one example being a homodimer. Other oligomers include heterodimers, homotrimers, heterotrimers, homotetramers, heterotetramers, etc.

One embodiment is directed to oligomers comprising multiple antigen binding proteins joined via covalent or non-covalent interactions between peptide moieties fused to the antigen binding proteins. Such peptides may be peptide linkers (spacers), or peptides that have the property of promoting oligomerization. Leucine zippers and certain polypeptides derived from antibodies are among the peptides that can promote oligomerization of antigen binding proteins attached thereto, as described in more detail below.

In particular embodiments, the oligomers comprise from two to four antigen binding proteins. The antigen binding proteins of the oligomer may be in any form, such as any of the forms described above, e.g., variants or fragments. Preferably, the oligomers comprise antigen binding proteins that have c-MPL receptor binding activity.

In one embodiment, an oligomer is prepared using polypeptides derived from immunoglobulins. Preparation of fusion proteins comprising certain heterologous polypeptides fused to various portions of antibody-derived polypeptides (including the Fc domain) has been described, e.g., by Ashkenazi et al., 1991, PNAS USA 88:10535; Byrn et al., 1990, Nature 344:677; and Hollenbaugh et al., 1992 “Construction of Immunoglobulin Fusion Proteins”, in Current Protocols in Immunology, Suppl. 4, pages 10.19.1-10.19.11.

One embodiment of the present invention is directed to a dimer comprising two fusion proteins created by fusing a c-MPL receptor binding fragment of an anti-c-MPL receptor antibody to the Fc region of an antibody. The dimer can be made by, for example, inserting a gene fusion encoding the fusion protein into an appropriate expression vector, expressing the gene fusion in host cells transformed with the recombinant expression vector, and allowing the expressed fusion protein to assemble much like antibody molecules, whereupon interchain disulfide bonds form between the Fc moieties to yield the dimer.

The term “Fc polypeptide” as used herein includes native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns.

One suitable Fc polypeptide, described in PCT application WO 93/10151 (hereby incorporated by reference), is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., 1994, EMBO J. 13:3992-4001. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

In other embodiments, the variable portion of the heavy and/or light chains of an anti-c-MPL receptor antibody may be substituted for the variable portion of an antibody heavy and/or light chain.

Alternatively, the oligomer is a fusion protein comprising multiple antigen binding proteins, with or without peptide linkers (spacer peptides). Among the suitable peptide linkers are those described in U.S. Pat. Nos. 4,751,180 and 4,935,233.

Another method for preparing oligomeric antigen binding proteins involves use of a leucine zipper. Leucine zipper domains are peptides that promote oligomerization of the proteins in which they are found. Leucine zippers were originally identified in several DNA-binding proteins (Landschulz et al., 1988, Science 240:1759), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., 1994, FEBS Letters 344:191, hereby incorporated by reference. The use of a modified leucine zipper that allows for stable trimerization of a heterologous protein fused thereto is described in Fanslow et al., 1994, Semin. Immunol. 6:267-78. In one approach, recombinant fusion proteins comprising an anti-c-MPL receptor antibody fragment or derivative fused to a leucine zipper peptide are expressed in suitable host cells, and the soluble oligomeric anti-c-MPL receptor antibody fragments or derivatives that form are recovered from the culture supernatant.

In another embodiment, the antibody derivatives can comprise at least one of the CDRs disclosed herein. For example, one or more CDR may be incorporated into known antibody framework regions (IgG1, IgG2, etc.), or conjugated to a suitable vehicle to enhance the half-life thereof. Suitable vehicles include, but are not limited to Fc, albumin, transferrin, and the like. These and other suitable vehicles are known in the art. Such conjugated CDR peptides may be in monomeric, dimeric, tetrameric, or other form. In one embodiment, one or more water-soluble polymer is bonded at one or more specific position, for example at the amino terminus, of a binding agent. In an example, an antibody derivative comprises one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative comprises one or more of monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers. In certain embodiments, one or more water-soluble polymer is randomly attached to one or more side chains. In certain embodiments, PEG can act to improve the therapeutic capacity for a binding agent, such as an antibody. Certain such methods are discussed, for example, in U.S. Pat. No. 6,133,426, which is hereby incorporated by reference for any purpose.

It will be appreciated that an antibody of the present invention may have at least one amino acid substitution, providing that the antibody retains binding specificity. Therefore, modifications to the antibody structures are encompassed within the scope of the invention. These may include amino acid substitutions, which may be conservative or non-conservative, that do not destroy the human c-MPL receptor binding capability of an antibody. Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties. A conservative amino acid substitution may also involve a substitution of a native amino acid residue with a normative residue such that there is little or no effect on the polarity or charge of the amino acid residue at that position.

Non-conservative substitutions may involve the exchange of a member of one class of amino acids or amino acid mimetics for a member from another class with different physical properties (e.g. size, polarity, hydrophobicity, charge). Such substituted residues may be introduced into regions of the human antibody that are homologous with non-human antibodies, or into the non-homologous regions of the molecule.

Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change may be avoided. In other words, based on information gathered from such routine experiments, one skilled in the art can readily determine the amino acids where further substitutions should be avoided either alone or in combination with other mutations.

A skilled artisan will be able to determine suitable variants of the polypeptide as set forth herein using well-known techniques. In certain embodiments, one skilled in the art may identify suitable areas of the molecule that may be changed without destroying activity by targeting regions not believed to be important for activity. In certain embodiments, one can identify residues and portions of the molecules that are conserved among similar polypeptides. In certain embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues which are important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of an antibody with respect to its three dimensional structure. In certain embodiments, one skilled in the art may choose not to make radical changes to amino acid residues predicted to be on the surface of the protein, since such residues may be involved in important interactions with other molecules.

A number of scientific publications have been devoted to the prediction of secondary structure. See Moult J., Curr. Op. in Biotech., 7(4):422-427 (1996), Chou et al., Biochemistry, 13(2):222-245 (1974); Chou et al., Biochemistry, 113(2):211-222 (1974); Chou et al., Adv. Enzymol. Relat. Areas Mol. Biol., 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural database (PDB) has provided enhanced predictability of secondary structure, including the potential number of folds within a polypeptide's or protein's structure. See Holm et al., Nucl. Acid. Res., 27(1):244-247 (1999). It has been suggested (Brenner et al., Curr. Op. Struct. Biol., 7(3):369-376 (1997)) that there are a limited number of folds in a given polypeptide or protein and that once a critical number of structures have been resolved, structural prediction will become dramatically more accurate.

Additional methods of predicting secondary structure include “threading” (Jones, D., Curr. Opin. Struct. Biol., 7(3):377-87 (1997); Sippl et al., Structure, 4(1):15-19 (1996)), “profile analysis” (Bowie et al., Science, 253:164-170 (1991); Gribskov et al., Meth. Enzym., 183:146-159 (1990); Gribskov et al., Proc. Nat. Acad. Sci., 84(13):4355-4358 (1987)), and “evolutionary linkage” (See Holm, supra (1999), and Brenner, supra (1997)).

In certain embodiments, variants of antibodies include glycosylation variants wherein the number and/or type of glycosylation site has been altered compared to the amino acid sequences of a parent polypeptide. In certain embodiments, variants comprise a greater or a lesser number of N-linked glycosylation sites than the native protein. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred antibody variants include cysteine variants wherein one or more cysteine residues are deleted from or substituted for another amino acid (e.g., serine) as compared to the parent amino acid sequence. Cysteine variants may be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

Desired amino acid substitutions (whether conservative or non-conservative) can be determined by those skilled in the art at the time such substitutions are desired. In certain embodiments, amino acid substitutions can be used to identify important residues of antibodies to human c-MPL receptor, or to increase or decrease the affinity of the antibodies to human c-MPL receptor described herein.

According to certain embodiments, preferred amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physiochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

In certain embodiments, antibodies of the invention may be chemically bonded with polymers, lipids, or other moieties.

The antigen binding agents may comprise at least one of the CDRs described herein incorporated into a biocompatible framework structure. In one example, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions or substitutions of amino acids, relative to a naturally occurring polypeptide or fold. These scaffolds can be derived from a polypeptide of any species (or of more than one species), such as a human, other mammal, other vertebrate, invertebrate, plant, bacteria or virus.

Typically the biocompatible framework structures are based on protein scaffolds or skeletons other than immunoglobulin domains. For example, those based on fibronectin, ankyrin, lipocalin, neocarzinostain, cytochrome b, CP1 zinc finger, PST1, coiled coil, LACI-D1, Z domain and tendamistat domains may be used (See e.g., Nygren and Uhlen, 1997, Current Opinion in Structural Biology, 7, 463-469).

Additionally, one skilled in the art will recognize that suitable binding agents include portions of these antibodies, such as one or more of heavy chain CDR1, CDR2, CDR3, light chain CDR1, CDR2 and CDR3 as specifically disclosed herein. At least one of the regions of heavy chain CDR1, CDR2, CDR3, CDR1, CDR2 and CDR3 may have at least one amino acid substitution, provided that the antibody retains the binding specificity of the non-substituted CDR. The non-CDR portion of the antibody may be a non-protein molecule, wherein the binding agent cross-blocks the binding of an antibody disclosed herein to human c-MPL and/or inhibits the activity of TPO signalling through the receptor. The non-CDR portion of the antibody may be a non-protein molecule in which the antibody exhibits a similar binding pattern to human MPL peptides in a competition binding assay as that exhibited by at least one of antibodies A1-A6, and/or neutralizes the activity of c-MPL receptor. The non-CDR portion of the antibody may be composed of amino acids, wherein the antibody is a recombinant binding protein or a synthetic peptide, and the recombinant binding protein cross-blocks the binding of an antibody disclosed herein to human c-MPL and/or neutralizes c-MPL receptor activity in vitro or in vivo. The non-CDR portion of the antibody may be composed of amino acids, wherein the antibody is a recombinant antibody, and the recombinant antibody exhibits a similar binding pattern to human c-MPL peptides in a competition binding assay as exhibited by at least one of the antibodies A1-A6, and/or neutralizes c-MPL receptor signalling.

### Nucleic Acids

In one aspect, the present invention provides isolated nucleic acid molecules that encode the antigen binding agents of the present invention. In addition, provided are vectors comprising the nucleic acids, cell comprising the nucleic acids, and methods of making the antigen binding proteins of the invention. The nucleic acids comprise, for example, polynucleotides that encode all or part of an antigen binding protein, for example, one or both chains of an antibody of the invention, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing. The nucleic acids can be any length. They can be, for example, 5, 10, 15, 20, 25, 30, 35, 40, 45, 50, 75, 100, 125, 150, 175, 200, 250, 300, 350, 400, 450, 500, 750, 1,000, 1,500, 3,000, 5,000 or more nucleotides in length, and/or can comprise one or more additional sequences, for example, regulatory sequences, and/or be part of a larger nucleic acid, for example, a vector. The nucleic acids can be single-stranded or double-stranded and can comprise RNA and/or DNA nucleotides, and artificial variants thereof (e.g., peptide nucleic acids).

Nucleic acids encoding antibody polypeptides (e.g., heavy or light chain, variable domain only, or full length) may be isolated from B-cells of mice that have been immunized with human c-MPL antigen. The nucleic acid may be isolated by conventional procedures such as polymerase chain reaction (PCR).

Nucleic acid sequences encoding the variable regions of the heavy and light chain variable regions are shown above. The skilled artisan will appreciate that, due to the degeneracy of the genetic code, each of the polypeptide sequences disclosed herein is encoded by a large number of other nucleic acid sequences. The present invention provides each degenerate nucleotide sequence encoding each antigen binding protein of the invention.

The invention further provides nucleic acids that hybridize to other nucleic acids (e.g., nucleic acids comprising a nucleotide sequence of any of A1-A14) under particular hybridization conditions. Methods for hybridizing nucleic acids are well-known in the art. See, e.g., Current Protocols in Molecular Biology, John Wiley & Sons, N.Y. (1989), 6.3.1-6.3.6. As defined herein, for example, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C., in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequences that are at least 65, 70, 75, 80, 85, 90, 95, 98 or 99% identical to each other typically remain hybridized to each other. The basic parameters affecting the choice of hybridization conditions and guidance for devising suitable conditions are set forth by, for example, Sambrook, Fritsch, and Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA. Changes can be introduced by mutation into a nucleic acid, thereby leading to changes in the amino acid sequence of a polypeptide (e.g., an antigen binding protein) that it encodes. Mutations can be introduced using any technique known in the art. In one embodiment, one or more particular amino acid residues are changed using, for example, a site-directed mutagenesis protocol. In another embodiment, one or more randomly selected residues is changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. In one embodiment, a nucleotide sequence provided herein for L-1 to L-6 and H-1 to H-6, or a desired fragment, variant, or derivative thereof, is mutated such that it encodes an amino acid sequence comprising one or more deletions or substitutions of amino acid residues that are shown herein for L-1 to L-6 and H-1 to H-6 to be residues where two or more sequences differ. In another embodiment, the mutagenesis inserts an amino acid adjacent to one or more amino acid residues shown herein for L-1 to L-6 and H-1 to H-6 to be residues where two or more sequences differ. Alternatively, one or more mutations can be introduced into a nucleic acid that selectively change the biological activity. (e.g., binding to human c-MPL) of a polypeptide that it encodes. For example, the mutation can quantitatively or qualitatively change the biological activity. Examples of quantitative changes include increasing, reducing or eliminating the activity. Examples of qualitative changes include changing the antigen specificity of an antigen binding protein.

In another aspect, the present invention provides nucleic acid molecules that are suitable for use as primers or hybridization probes for the detection of nucleic acid sequences of the invention. A nucleic acid molecule of the invention can comprise only a portion of a nucleic acid sequence encoding a full-length polypeptide of the invention, for example, a fragment that can be used as a probe or primer or a fragment encoding an active portion (e.g., a human c-MPL binding portion) of a polypeptide of the invention.

Probes based on the sequence of a nucleic acid of the invention can be used to detect the nucleic acid or similar nucleic acids, for example, transcripts encoding a polypeptide of the invention. The probe can comprise a label group, e.g., a radioisotope, a fluorescent compound, an enzyme, or an enzyme co-factor. Such probes can be used to identify a cell that expresses the polypeptide.

In another aspect, the present invention provides vectors comprising a nucleic acid encoding a polypeptide of the invention or a portion thereof. Examples of vectors include, but are not limited to, plasmids, viral vectors, non-episomal mammalian vectors and expression vectors, for example, recombinant expression vectors.

The recombinant expression vectors of the invention can comprise a nucleic acid of the invention in a form suitable for expression of the nucleic acid in a host cell. The recombinant expression vectors include one or more regulatory sequences, selected on the basis of the host cells to be used for expression, which is operably linked to the nucleic acid sequence to be expressed. Regulatory sequences include those that direct constitutive expression of a nucleotide sequence in many types of host cells (e.g., SV40 early gene enhancer, Rous sarcoma virus promoter and cytomegalovirus promoter), those that direct expression of the nucleotide sequence only in certain host cells (e.g., tissue-specific regulatory sequences, see Voss et al., 1986, Trends Biochem. Sci. 11:287, Maniatis et al., 1987, Science 236:1237, incorporated by reference herein in their entireties), and those that direct inducible expression of a nucleotide sequence in response to particular treatment or condition (e.g., the metallothionin promoter in mammalian cells and the tet-responsive and/or streptomycin responsive promoter in both prokaryotic and eukaryotic systems (see id.). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

In another aspect, the present invention provides host cells into which a recombinant expression vector of the invention has been introduced. A host cell can be any prokaryotic cell or eukaryotic cell. Prokaryotic host cells include gram negative or gram positive organisms, for example E. coli or bacilli. Higher eukaryotic cells include insect cells, yeast cells, and established cell lines of mammalian origin. Examples of suitable mammalian host cell lines include Chinese hamster ovary (CHO) cells or their derivatives such as Veggie CHO and related cell lines which grow in serum-free media (see Rasmussen et al., 1998, Cytotechnology 28:31) or CHO strain DXB-11, which is deficient in DHFR (see Urlaub et al., 1980, Proc. Natl. Acad. Sci. USA 77:4216-20). Additional CHO cell lines include CHO-K¹ (ATCC#CCL-61), EM9 (ATCC#CRL-1861), and UV20 (ATCC#CRL-1862). Additional host cells include the COS-7 line of monkey kidney cells (ATCC CRL 1651) (see Gluzman et al., 1981, Cell 23:175), L cells, C127 cells, 3T3 cells (ATCC CCL 163), AM-1/D cells (described in U.S. Pat. No. 6,210,924), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Appropriate cloning and expression vectors for use with bacterial, fungal, yeast, and mammalian cellular hosts are described by Pouwels et al. (Cloning Vectors: A Laboratory Manual, Elsevier, N.Y., 1985).

Vector DNA can be introduced into prokaryotic or eukaryotic cells via conventional transformation or transfection techniques. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die), among other methods.

The transformed cells can be cultured under conditions that promote expression of the polypeptide, and the polypeptide recovered by conventional protein purification procedures. One such purification procedure is described in the Examples below. Polypeptides contemplated for use herein include substantially homogeneous recombinant mammalian anti-c-MPL receptor antibody polypeptides substantially free of contaminating endogenous materials.

Cells containing the nucleic acid encoding the antigen binding proteins of the present invention also include hybridomas. The production and culturing of hybridomas are discussed in the antibody section above.

### Activity of Antigen Binding Proteins

In one aspect, the present invention provides antigen binding proteins, in particular human, humanized, chimeric or murine antibodies, that specifically bind to the human c-MPL receptor. Such antibodies include antagonizing or neutralizing antibodies capable of reducing or neutralizing TPO-Receptor-Mediated signalling, as determined, for example, by the cell based functional assay described in Example 4 herein. In another embodiment, the antigen binding proteins, such as, e.g., the murine anti-human antibodies of the present invention and the like, have an IC50 value of less than or equal to: 90 nM; 80 nM; 70 nM; 60 nM; 50 nM; 40 nM; 30 nM; 20 nM; 10 nM; 5 nM; 4 nM; 3 nM; 2 nM; 1 nM (e.g., 1000 pM); 900, pM; 800 pM; 700 pM; 600 pM; 500 pM; 400 pM; 300 pM; 200 pM; 100 pM; 50 pM; 25 pM; 20 pM; 15 pM; 10 pM; 5 pM; 3 pM or 1 pM. In another embodiment, the antigen binding proteins such as the human antibodies of the present invention are capable of specifically binding to the human c-MPL receptor, and have an IC50 value that is substantially similar to that of a reference antibody. In another embodiment, the antigen binding proteins have a Kb (or Kd) as measured by the assay described in the Examples herein (or similar assays), that is substantially similar to that of a reference antibody. As used herein, the term “substantially similar” means comparable to, or about 100%, 99%, 98%, 97%, 95%, 90%, 85%, 80%, 75%, 70%, 65% or 50% identical to the IC50 or Kb (or Kd) value of the reference antibody. Reference antibodies include, for example, antibodies having a combination of heavy chain and light chains L1H1, L2H2, L3H3, L4H4, L5H5, and L6H6. In one embodiment, the reference antibodies include A-1, A-2, A-3, A-4, A-5, and A-6.

In one embodiment, the invention antigen binding proteins, such as the human or murine-anti-human c-MPL antibodies herein, bind to c-MPL with a Kd value in the range of 1 pM up to about 100 nM; 90; nM; 80 nM; 70 nM; 60 nM; 50 nM; 40 nM; 30 nM; 20 nM; 10 nM; 5 nM; 4 nM; 3 nM; 2 nM; 1 nM (e.g., 1000 pM); 900, pM; 800 pM; 700 pM; 600 pM; 500 pM; 400 pM; 300 pM; 200 pM; 100 pM; 50 pM; 25 pM; 20 pM; 15 pM; 10 pM; or 5 pM. In particular embodiments, the Kd value of the antigen binding protein is less than or equal to: 90; nM; 80 nM; 70 nM; 60 nM; 50 nM; 40 nM; 30 nM; 20 nM; 10 nM; 5 nM; 4 nM; 3 nM; 2 nM; 1 nM (e.g., 1000 pM); 900, pM; 800 pM; 700 pM; 600 pM; 500 pM; 400 pM; 300 pM; 200 pM; 100 pM; 50 pM; 25 pM; 20 pM; 15 pM; 10 pM; 5 pM; 3 pM or 1 pM. As is well-known in the art, the higher the binding affinity is of a particular antigen binging protein for its cognate receptor (e.g., the invention mAbs provided herein binding to c-MPL), the lower the Kd value will be for the respective antibody.

Binding to Human c-MPL Receptor

In one embodiment, the present invention provides antigen binding proteins that cross-competes for binding with a reference antibody, wherein the reference antibody comprises a combination of light chain and heavy chain variable domain sequences selected from the group consisting of L1H1, L2H2, L3H3, L4H4, L5H5, and L6H6. In another embodiment, the present invention provides human antibodies that cross-compete for binding with a reference antibody, wherein the reference antibody is selected from at least one of A-1, A-2, A-3, A-4, A-5, or A-6. In another aspect, the present invention provides human or murine-anti-human antibodies that bind to the extracellular region of the human c-MPL receptor. In another embodiment, the present invention provides anti-human antibodies that cross-compete for binding with a reference antibody, wherein the reference antibody binds to the extracellular region of the human c-MPL receptor. In another embodiment, the present invention provides human or murine anti-human antibodies that bind to extracellular region of the c-MPL receptor, and have an IC50 value of the antigen binding protein that is less than or equal to: 90 nM; 80 nM; 70 nM; 60 nM; 50 nM; 40 nM; 30 nM; 20 nM; 10 nM; 5 nM; 4 nM; 3 nM; 2 nM; 1 nM (e.g., 1000 pM); 900, pM; 800 pM; 700 pM; 600 pM; 500 pM; 400 pM; 300 pM; 200 pM; 100 pM; 50 pM; 25 pM; 20 pM; 15 pM; 10 pM; 5 pM; 3 pM or 1 pM, as determined, for example, in the assay set out in Example 5. In another embodiment, the present invention provides human antibodies that cross-compete for binding to the human c-MPL receptor with a reference antibody, wherein the reference antibody is selected from at least one of A-1 to A-6 (see Table 4).

In a further embodiment, the antigen binding proteins, when bound to the human c-MPL receptor binds to the human c-MPL receptor with substantially the same Kd as a reference antibody; inhibits TPO stimulation of the human c-MPL receptor with substantially the same IC50 as said reference antibody; and/or cross-competes for binding with said reference antibody on human c-MPL receptor, wherein said reference antibody comprises a combination of light chain and heavy chain variable domain sequences selected from the group consisting of L1H1, L2H2, L3H3, L4H4, L5H5, and L6H6.

In a further embodiment, an isolated human antibody is provided that, when bound to the human c-MPL receptor: binds to the human c-MPL receptor with substantially the same Kd as a reference antibody; inhibits TPO stimulation of the human c-MPL receptor with substantially the same IC₅₀ as said reference antibody; and/or cross-competes for binding with said reference antibody on human c-MPL receptor, wherein said reference antibody is selected from the group consisting of A-1, A-2, A-3, A-4, A-5, and A-6.

The ability to cross-compete with an antibody can be determined using any suitable assay, such as the Biacore assay described in the Examples herein, or another exemplary competitive binding assay using any one of A-1 to A-6 as the reference antibody. The results of such a Biacore cross-competition assays are shown in Table 4. It has been found that in particular embodiments, the invention mAbs compete with the native human TPO protein. Invention anti-human c-MPL mAbs that are believed to fall into this category include, for example, mAbs 1.169; 1.36 and 1.78 (e.g., from Epitope Bin I), and the like. In certain other embodiments, invention mAbs are believed to not compete with native human TPO for binding to the c-MPL receptor, and include for example, mAbs 1.6, 1.75 and 1.111 (e.g., from Epitope Bin II), and the like.

TABLE 4
Properties of Anti-hu-c-Mpl MAbs

| | | Epitope | | KD |
|---|---|---|---|---|
| Antibody | Activity | Bin* | Isotype | (95% confidence limits) pM |
| mAb 1.75 | Neutralizing | I | IgG2bκ | 10 (6-16) |
| mAb 1.6 | Neutralizing | I | IgG2bκ | 36 (25-52) |
| mAb1.111 | Neutralizing | I | IgG1κ | 36 (18-56) |
| mAb 1.78 | Neutralizing | II | IgG1κ | 72 (56-90) |
| mAb 1.36 | Neutralizing | II | IgG1κ | 450 (320-610) |
| mAb | Neutralizing | II | IgG1κ | 480 (390-570) |
| 1.169 | | | | |

*Based on Biacore competition and neutralization studies

It has also been found herein, using the well-known Pepspot methods (Reineke et al., Curr Top Microbiol Immunol (1999)243:23-36, incorporated herein by reference), that the invention antibodies from Epitope Bin I (e.g., mAb 1.75; mAb 1.6; mAb1.111; and the like) bind to the epitope sequence corresponding to amino acids 217-223 of the c-mpl receptor (SEQ ID NO:99), which epitope corresponds to -PWQDGPK- (SEQ ID NO:97).

As set forth herein, novel anti-hu-c-Mpl mAbs are provided herein that show binding specificity for human c-Mpl, in for example, flow cytometry studies and the like. Two of these mAbs in particular, 1.6 and 1.75, exhibited good sensitivity and bound specifically to various cells that express human c-Mpl. Human and murine cells engineered to express high levels of exogenous human c-Mpl, immortalized human hematopoietic cell lines that express moderate levels of the receptor (up to several thousand receptors/cell), and human platelets that express low levels (20-200 receptors/platelet) of c-Mpl were all readily stained by these mAbs. The level of binding observed at a particular antibody concentration and the shift in mean fluorescence intensity was consistent with these particular antibody's (e.g., 1.6 and 1.75) pM affinities and correlated with the levels of c-Mpl mRNA in the 13 cell lines tested. The lack of staining of KG-1 cells, which have only very low levels of c-Mpl mRNA, could be due to the lack of surface expression of the c-Mpl protein and/or the detection limit of these mAbs. The specificity of mAbs 1.6 and 1.75 was demonstrated by the lack of binding to any of the 7 c-Mpl⁻ cell lines tested, as well as the fact that surface binding to c-Mpl⁺ cells was significantly reduced when c-Mpl mRNA expression was knocked down with c-Mpl-specific siRNAs or when the mAbs were pre-incubated with soluble c-Mpl protein.

In line with knockout studies demonstrating the essential role of c-Mpl in murine hematopoietic stem cell self-renewal and proliferation, and thus the inferred expression of c-Mpl on the surface of hematopoietic stem and progenitor cells, invention mAbs 1.6 and 1.75 demonstrated that a significant proportion of human CD34⁺ cells isolated from bone marrow or mobilized peripheral blood express c-Mpl on the cell surface. The data provided herein further suggest that there are four separate populations of CD34⁺ cells that differ in their level of c-Mpl and CD61 expression. The c-Mpl⁺CD61⁺ and c-Mpl⁻CD61⁺ subpopulations appear to be rare, representing ˜2-4% of total CD34⁺ cells. The c-Mpl⁻CD61⁻ and c-Mpl⁺CD61⁻ subpopulations are more prevalent, ranging from 45-85% and 10-50%, respectively, in samples from different sources and obtained from different donors.

### Indications

Abdel-Azim et al. reported data demonstrating that dimerization of a single c-MPL cytokine receptor can deliver a profound expansion signal in both uncommitted and lymphoid-committed human hematopoietic progenitors (Blood 2008 Apr. 15; 111(8):4064-74). Ramsfiell et al. demonstrated that thrombopoietin (TPO), via its cognate c-MPL receptor, directly and potently stimulates multilineage growth and progenitor cell expansion from primitive (CD34+ CD38−) human bone marrow progenitor cells; in addition, Ramsfiell et al. also teach that Tpo-recruited CD34+ CD38− progenitor cells have a multilineage differentiation potential, and that Tpo promotes prolonged expansion of multipotent progenitors (J. Immunol. 1997 Jun. 1; 158(11):5169-77.). In addition, Jacobsen et al. in a review, indicate that TPO [via the c-MPL receptor] has effects on primitive hematopoietic progenitor cells (Stem Cells 1996; 14 Suppl 1:173-80). When acting alone, TPO stimulates little or no growth, but promotes viability and suppresses apoptosis of murine multipotent (Lin− Sca-1+) bone marrow progenitor cells in vitro. In addition, TPO directly and potently synergizes with other early acting cytokines (kit ligand, flt3 ligand and interleukin 3) to promote multilineage growth of the same progenitor cell population (Stem Cells 1996; 14 Suppl 1:173-80). The MPL receptor was found at the mRNA and/or protein level in 40-80% of primary acute myeloid leukemia (AML) cases in various series; MPL expression was not limited to certain morphological FAB types, although the highest percentages were seen in the M6 (erythroid) and M7 (megakaryocytic) subclasses (Drexler & Quentmeier, Leukemia 1996 September; 10(9):1405-21). A significant portion of AML cases and of erythroid, megakaryocytic and myeloid leukemia cell lines co-expressed TPO and MPL mRNA transcripts, although no biologically active TPO appeared to be secreted by these cells (Drexler 1996). In several studies TPO induced in vitro proliferation of 14-37% of primary AML cases, predominantly of the M2 and M7 subtypes (Drexler 1996). TPO significantly enhanced the cytokine-induced growth of AML cells in a substantial fraction of cases responsive to GM-CSF, IL-3, IL-6 or SCF (Drexler 1996). Matsumura et al., report that the proliferative responses of AML cells to TPO were observed not only in M7-type, but also in the other subtypes of AML cases (Leuk Lymphoma 1996 November; 23(5-6):533-8). Furthermore, the TPO-induced proliferation of AML cells was augmented by the addition of the other hematopoietic growth factors such as interleukin-3 (IL-3), IL-6, stem cell factor, or granulocyte-macrophage colony-stimulating factor (Matsumura 1996). In addition to proliferation, TPO appeared to induce megakaryocytic differentiation in a small part of AML cells (Matsumura 1996). Matsumura et al. indicate that these results suggested that TPO/c-mpl system might contribute, at least in part, to abnormal growth and differentiation of AML cells (Matsumura 1996).

In addition Drexler et al. also developed an in vitro system which allowed questions regarding the biology of TPO to be addressed (Leukemia 1997 April; 11(4):541-51). Drexler et al. found that although the acute myeloid leukemia (AML)-derived cell lines HU-3, M-07e, M-MOK and TF-1 have absolute dependence on granulocyte-macrophage colony-stimulating factor (GM-CSF), when these cell lines were cultured long term (>6 months) in the continuous presence of TPO (omitting GM-CSF), that TPO alone supported the maintenance and expansion of these sister cell lines, HU-3/TPO, M-07e/TPO, M-MOK/TPO and TF-1/TPO, which displayed somewhat longer doubling times, a larger cell size, and a higher percentage of polynucleated giant cells and slightly adherent cells than the corresponding countercultures grown with GM-CSF (Drexler 1997). Drexler et al. found that in the absence of TPO the cells died quickly, within a few days; indicating that the TPO-grown cell lines have an absolute dependence on the TPO factor; but could all be switched back to growth with GM-CSF. Drexler et al. concluded that in long-term exposure, TPO appears to have both a proliferative and a differentiative effect on responsive cells; and that under serum-deprived culture conditions, TPO acted as a survival factor on the TPO-cell lines (Drexler 1997).

Matsamura et al. found that c-mpl expression and proliferative response to rhTPO was observed in all subtypes of AML and did not correlate with French-American-British classification, whereas all cases of M7-type AML cells expressed c-mpl and proliferated in response to rhTPO (Blood 1995 Jul. 15; 86(2):703-9.). Furthermore, Matsamura et al. found that rhTPO-induced proliferation of AML cells was augmented with the addition of interleukin-3 (IL-3), IL-6, stem cell factor, or granulocyte-macrophage colony-stimulating factor (Matsumura 1995). Matsamura et al. concluded that these results suggested that c-mpl may be functional in terms of supporting proliferation of various types of AML cells and that TPO may contribute, at least in part, to abnormal growth of the cells, especially in combination with other hematopoietic growth factors (Matsamura 1995).

Accordingly, the antagonistic invention anti-c-MPL antibodies provided herein can be used to inhibit (e.g., antagonize or neutralize) the TPO and/or c-MPL mediated expansion signal in both uncommitted and lymphoid-committed human hematopoietic progenitors. In another embodiment, the invention antibodies can be used to inhibit (e.g., antagonize or neutralize) the TPO and/or c-MPL mediated stimulation of multilineage growth and progenitor cell expansion from primitive (CD34+ CD38−) human bone marrow progenitor cells. In another embodiment, the invention antibodies can be used to inhibit (e.g., antagonize or neutralize) the TPO and/or c-MPL mediated suppression of apoptosis of mammalian multipotent (e.g., murine Lin− Sca-1+; human and the like) bone marrow progenitor cells. In another embodiment, the invention antibodies can be used to inhibit (e.g., antagonize or neutralize) the TPO and/or c-MPL mediated promotion of multilineage growth of mammalian multipotent (e.g., murine Lin− Sca-1+; human and the like) bone marrow progenitor cells. In yet another embodiment, the invention antibodies can be used to inhibit (e.g., antagonize or neutralize) the TPO induced maintenance and/or proliferation of primary AML cells, such as e.g., the M2 and M7 subtypes. In another embodiment, the invention antibodies can be used to inhibit (e.g., antagonize or neutralize) the TPO and/or c-MPL mediated enhancement of cytokine-induced growth of AML cells. In another embodiment, the invention antibodies can be used to inhibit (e.g., antagonize or neutralize) the TPO and/or c-MPL mediated induction of megakaryocytic differentiation in a portion of AML cells. In another embodiment, the invention antibodies can be used to inhibit (e.g., antagonize or neutralize) the TPO and/or c-MPL mediated proliferation of AML cells augmented or enhanced by the addition of the other hematopoietic growth factors such as interleukin-3 (IL-3), IL-6, stem cell factor (SCF), or granulocyte-macrophage colony-stimulating factor (GM-CSF). In another embodiment, the invention antibodies can be used to inhibit (e.g., antagonize or neutralize) the TPO and/or c-MPL mediated survival effect on TPO-dependent AML-derived cell lines (e.g., or primary AML cells. In another embodiment, the invention antibodies can be used to inhibit (e.g., antagonize or neutralize) the TPO and/or c-MPL mediated proliferative and a differentiative effect on TPO-responsive cells.

### Methods of Treatment

In another aspect, a method of treating a subject, comprising administering a therapeutic dosage of the antigen binding proteins of the present invention is provided. In one embodiment, the antigen binding proteins are murine or human anti-c-MPL antibodies. As used herein the term “subject” refers to a mammal, including humans, and is used interchangeably with the term “patient.” The invention antibodies provided herein, including murine or human anti-c-MPL Mabs and the like, can be used to treat, control or prevent a disorder (e.g., disease) or condition characterized by undesired, uncontrolled and/or uninhibited TPO and/or c-MPL mediated bioactivity in a subject (such as those bioactivities described above), such as the activity resulting from the binding of TPO to c-MPL, and the like.

These disorders or diseases include myeloproliferative neoplasms (MPN) or myeloproliferative diseases (MPD); such as for example, polycythemia vera, essential thrombocythemia, primary myelofibrosis, chronic myelogenous leukemia (CML) or acute myelogenous leukemia (AML), and the like.

The myeloproliferative neoplasms (MPNs), unlike MDS, usually exhibit terminal myeloid cell expansion in the peripheral blood. The MPNs include polycythemia vera, essential thrombocytosis, chronic myelogenous leukemia (CML), and primary myelofibrosis (PMF). Other diseases contemplated for treatment herein include Atypical MPNs, such as those chronic myeloid disorders which are currently not classifiable as either MDS or classical MPN, such as for example: chronic myelomonocytic leukemia, juvenile myelomonocytic leukemia, systemic mastocytosis, hypereosinophilic syndrome, chronic neutrophilic leukemia, chronic eosinophilic leukemia, chronic basophilic leukemia, and the like. Other diseases contemplated for treatment herein include diseases where the TPO/c-MPL axis is important and/or the c-MPL receptor is activated.

The term “treatment” encompasses alleviation or prevention of at least one symptom or other aspect of a disorder, or reduction of disease severity, and the like. An antigen binding protein, in particular a human antibody according to the present invention, need not effect a complete cure, or eradicate every symptom or manifestation of a disease, to constitute a viable therapeutic agent. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the invention is directed to a method comprising administering to a patient an antigen binding protein such as a human antibody in an amount and for a time sufficient to induce a sustained improvement over baseline of an indicator that reflects the severity of the particular disorder.

As is understood in the pertinent field, pharmaceutical compositions comprising the antigen binding proteins of the invention are administered to a subject in a manner appropriate to the indication and the composition. In one embodiment, pharmaceutical compositions comprise the human antibodies of the present invention. Pharmaceutical compositions may be administered by any suitable technique, including but not limited to parenterally, topically, or by inhalation. If injected, the pharmaceutical composition can be administered, for example, via intra-articular, intravenous, intramuscular, intralesional, intraperitoneal or subcutaneous routes, by bolus injection, or continuous infusion. Delivery by inhalation includes, for example, nasal or oral inhalation, use of a nebulizer, inhalation of the antigen binding protein in aerosol form, and the like. Other alternatives include oral preparations including pills, syrups, or lozenges.

Advantageously, the antigen binding proteins of the invention, are administered in the form of a composition comprising one or more additional components such as a physiologically acceptable carrier, excipient or diluent. Optionally, the composition additionally comprises one or more physiologically active agents, for example, as described below. In various particular embodiments, the composition comprises one, two, three, four, five, or six physiologically active agents in addition to one or more antigen binding proteins (e.g, human antibodies) of the present invention.

In one embodiment, the pharmaceutical composition comprises a human antibody of the invention together with one or more substances selected from the group consisting of a buffer suitable for antibodies at a suitable pH, an antioxidant such as ascorbic acid, a low molecular weight polypeptide (such as those having fewer than 10 amino acids), a protein, an amino acid, a carbohydrate such as dextrin, a chelating agent such as EDTA, glutathione, a stabilizer, and an excipient. In accordance with appropriate industry standards, preservatives may also be added. The composition may be formulated as a lyophilizate using appropriate excipient solutions as diluents. Suitable components are nontoxic to recipients at the dosages and concentrations employed. Further examples of components that may be employed in pharmaceutical formulations are presented in Remington's Pharmaceutical Sciences, 16^{th} Ed. (1980) and 20^{th} Ed. (2000), Mack Publishing Company, Easton, Pa.

Kits for use by medical practitioners are provided including one or more antigen binding proteins of the invention and a label or other instructions for use in treating any of the conditions discussed herein. In one embodiment, the kit includes a sterile preparation of one or more human antibodies, which may be in the form of a composition as disclosed above, and may be in one or more vials.

Dosages and the frequency of administration may vary according to such factors as the route of administration, the particular antibodies employed, the nature and severity of the disease to be treated, whether the condition is acute or chronic, and the size and general condition of the subject. Appropriate dosages can be determined by procedures known in the pertinent art, e.g. in clinical trials that may involve dose escalation studies.

An antigen binding protein, in particular, the human antibodies, of the invention may be administered, for example, once or more than once, e.g., at regular intervals over a period of time. In particular embodiments, a human antibody is administered over a period of at least once a month or more, e.g., for one, two, or three months or even indefinitely. For treating chronic conditions, long-term treatment is generally most effective. However, for treating acute conditions, administration for shorter periods, e.g. from one to six weeks, may be sufficient. In general, the human antibody is administered until the patient manifests a medically relevant degree of improvement over baseline for the chosen indicator or indicators.

One example of therapeutic regimens provided herein comprise subcutaneous injection of an antigen binding protein such as a human antibody once a week, or once every two weeks, at an appropriate dosage, to treat a condition in which it is desired to inhibit or neutralize TPO/c-MPL bioactivity, such as for example, AML or the like. Weekly or monthly administration of antigen binding protein would be continued until a desired result is achieved, e.g., the subject's symptoms subside. Treatment may resume as needed, or, alternatively, maintenance doses may be administered.

Particular embodiments of methods and compositions of the invention involve the use of an antigen binding protein such as a murine or human anti-c-MPL antibody and one or more c-MPL antagonists for example, two or more antigen binding proteins of the invention, or an antigen binding protein of the invention and one or more other c-MPL antagonists. In further embodiments, antigen binding protein are administered alone or in combination with other agents useful for treating the condition with which the patient is afflicted. Examples of such agents include both proteinaceous and non-proteinaceous drugs. When multiple therapeutics are co-administered, dosages may be adjusted accordingly, as is recognized in the pertinent art. “Co-administration” and combination therapy are not limited to simultaneous administration, but also include treatment regimens in which an antigen binding protein is administered at least once during a course of treatment that involves administering at least one other therapeutic agent to the patient.

### Diagnostic Methods and Kits

The invention further provides a method of determining the presence or absence of c-MPL) on a cell. The method includes: (a) contacting a sample with a c-MPL-specific monoclonal antibody, or a functional fragment thereof, under conditions sufficient for binding, and (b) measuring binding of the human c-MPL-specific monoclonal antibody, or functional fragment thereof, wherein binding of the c-MPL-specific monoclonal antibody, or functional fragment thereof, indicates the presence of c-MPL, and wherein an absence of binding of the c-MPL-specific monoclonal antibody, or functional fragment thereof, indicates the absence of c-MPL. The antibody can be a human c-MPL-specific monoclonal antibody (such as those set forth in Table 4, and the like) for detecting the presence or absence of human c-MPL on a human cell.

Any of the c-MPL-specific antibodies of the invention described herein can be employed in the method of determining the presence or absence of a c-MPL on a cell. These c-MPL-specific monoclonal antibodies exhibit specific binding activity toward c-MPL compared to prior art antibodies. This specificity can be beneficially used to detect the presence or absence of c-MPL in cell samples under a variety of conditions and for a wide variety of applications. Exemplary applications include determining the presence or absence of c-MPL on a particular cell type or within a particular tissue or organ. The presence of c-MPL can be used as a diagnostic marker for particular cell types such as hematopoietic (e.g., progenitor) cells. The presence of c-MPL also can be used for diagnostic purposes to indicate the applicability of therapeutic treatments.

For example, therapeutic treatments where it is desired to augment the proliferation, differentiation, migration and/or survival of a target cell type or target cell types (such as those described in the Indications section herein) can be benefited by knowing that c-MPL is present or absent on the target or non-target cell type. The presence of c-MPL on a target cell type or types will allow consideration of whether c-MPL stimulatory or inhibitory treatment would be beneficial, depending on the particular disease state.

Similarly, for example, knowing that c-MPL is present or absent on target or non-target cell types also is beneficial in therapeutic treatments where proliferation, differentiation, migration and/or survival of a target cell type is not desired. For example, the presence or absence of c-MPL on either or both cell categories will allow consideration of whether to evaluate the benefit of using a c-MPL stimulatory or inhibitory treatment as described above.

c-MPL antagonists including, for example, c-MPL-specific monoclonal antibodies having c-MPL antagonist activity provided herein can be administered to bind c-MPL and inhibit its activity. In particular, antagonistic c-MPL-specific monoclonal antibodies of the invention can be used for the therapeutic treatment of diseases where there is increased signaling mediated by TPO and/or c-MPL, as described herein. In these types of pathological conditions, an antagonistic c-MPL-specific monoclonal antibody of the invention can be used to diminish or block signaling by c-MPL and treat or ameliorate the disease. c-MPL inhibitory treatment of such conditions include, for example, administering an effective amount of a c-MPL antagonist, including a c-MPL-specific antagonistic monoclonal antibody of the invention, for a sufficient period of time to reduce the severity of the condition. c-MPL inhibitory treatments, including therapeutic antibody treatments, are well known in the art and can be similarly applied to the therapeutic methods of invention given the teachings and guidance provided herein. Such treatments can be administered in conjunction with any of a variety of formulations, pharmaceutically acceptable media and/or pharmaceutically acceptable carriers well known in the art.

With respect to use as a diagnostic marker, the presence of c-MPL can be used as a general marker for hematopoietic cell (e.g., megakaryocyte, platelets, and the like), tissue and/or organ identification. Determination of the presence or absence of c-MPL on a cell surface or within a cellular sample also can be combined with other diagnostic markers indicative of a particular cell, tissue or organ type to increase accuracy of the identification. The c-MPL-specific monoclonal antibodies of the invention can be specific to human c-MPL, where determination of the presence or absence of human c-MPL is desired, for example.

Given the teachings and guidance provided herein, those skilled in the art will understand that the methods of determining the presence or absence of c-MPL on a cell can be applied to single cells, cell populations, tissues or organs. The methods of determining the presence or absence of c-MPL on a cell also can be applied to cell lysates obtained from single cells, cell populations, tissues or organs. Such determinations are further useful to map c-MPL presence or absence at the cell, tissue or organ level as well as subcategories thereof. Identification of which physiological structures and substructures contain or lack c-MPL is beneficial for diagnostic and therapeutic purposes as described above.

The method of determining the presence or absence of c-MPL, including human c-MPL, includes contacting a sample with a c-MPL-specific monoclonal antibody of the invention, or functional fragment thereof. Contacting is performed under conditions sufficient for binding. Binding conditions for monoclonal antibodies and functional fragments thereof are well known in the art. Exemplary binding formats include, for example, solid phase, solution phase, cell surface binding, immunocytochemistry and in situ binding. These and any of a variety of other formats well known in the art can be employed in the method of the invention for determining the presence or absence of c-MPL. Such methods can be found described in, for example, Harlow and Lane, Antibodies: A Laboratory Manual, Cold Spring Harbor Laboratory, New York (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pliickthun and Skerra, Meth. Enzymol., 178:497-515 (1989) and in Day, E. D., Advanced Immunochemistry, Second Ed., Wiley-Liss, Inc., New York, N.Y. (1990). Specific examples of binding conditions and formats useful in the method of detecting c-MPL are described further below in the Examples and include Western blotting, flow cytometry and confocal microscopy.

Following contacting a sample with a c-MPL-specific antibody of the invention, the presence or absence of c-MPL can be determined by measuring the amount of c-MPL-specific binding to the sample. Specific binding to the sample indicates the presence of c-MPL in the sample. Non-specific binding or a lack of binding indicates the absence of c-MPL in the sample. Such measurements can be quantitative, qualitative and/or relative to a control sample. Similarly, the amount of c-MPL present in a sample indicates the abundance of c-MPL in the sample.

A method of determining susceptibility of a cell to c-MPL-mediated proliferation also is provided. The method includes: (a) contacting a cellular sample with a c-MPL-specific monoclonal antibody, or functional fragment thereof, under conditions sufficient for binding, and (b) measuring the binding of the c-MPL-specific monoclonal antibody, or functional fragment thereof, wherein the presence of c-MPL indicates susceptibility of the cellular sample to c-MPL-mediated proliferation. The method includes use of a human c-MPL-specific monoclonal antibody for determining the susceptibility of a human cellular sample to c-MPL-mediated proliferation.

As with the method of determining c-MPL presence or absence described above, any of the c-MPL-specific antibodies of the invention also can be employed in the method of determining the susceptibility of a cell to c-MPL-mediated proliferation. The c-MPL-specific monoclonal antibodies of the invention exhibit specific binding activity toward c-MPL compared to prior art antibodies, which can be used to accurately detect the presence or absence of c-MPL in a cellular sample. The presence of c-MPL on the cell surface within the sample indicates susceptibility of that cell to TPO-induced proliferation. Similarly, since TPO induces proliferation, differentiation, and/or cell survival of hematopoietic cells, the presence of c-MPL in the cellular sample also is indicative of those cells as being susceptible to TPO-induced proliferation, differentiation, and/or survival.

A cell or cellular sample determined to be susceptible to c-MPL-mediated cell proliferation (e.g. expansion), differentiation, or survival can be tested for these c-MPL-mediated activities by culturing the cells in the presence or absence of TPO (or another c-MPL agonist) and determining if the presence of TPO specifically induces one or more of these physiological responses. Susceptible cells, tissues or organs identified as responsive to a c-MPL-mediated activity can be considered as a therapeutic target or as non-target cell type as described previously. Therapeutic treatments utilizing a c-MPL (TPO-R) agonist can be designed or excluded based on the whether the target or non-target cell type or types exhibit a c-MPL-mediated proliferation, differentiation, migration or survival activity.

Given the teachings and guidance provided herein, those skilled in the art will understand that the methods of determining the susceptibility of a cell to c-MPL-mediated proliferation or other activity can be applied to single cells, cell lysates, cell populations, tissues or organs or subpopulations thereof. Identification of which physiological groupings of cells, tissues and/or organs are responsive to c-MPL-mediated activity is beneficial for diagnostic and therapeutic purposes as described above.

The method of determining the susceptibility of a cell to c-MPL-mediated proliferation or other activity, including human c-MPL, includes contacting a cell sample with a c-MPL-specific monoclonal antibody of the invention, or functional fragment thereof. Contacting is performed under conditions sufficient for binding to the cell, tissue or organ surface. Binding conditions for monoclonal antibodies and functional fragments thereof are well known in the art. Exemplary binding formats include, for example, solid phase, solution phase, cell surface binding, immunocytochemistry and in situ binding as exemplified previously. Similarly, as described previously, these and any of a variety of other formats and binding conditions well known in the art can be employed in the method of the invention for determining the susceptibility of a cell to a c-MPL-mediated activity. Specific examples of binding conditions and formats that can be used in this method of the invention are described further below in the Examples and include Western blotting, flow cytometry and confocal microscopy.

Following contacting a sample with a c-MPL-specific antibody of the invention, the presence or absence of c-MPL can be determined by measuring the amount of c-MPL-specific binding to the sample as described previously. Presence of c-MPL correlates with susceptibility of a cellular sample to a c-MPL-mediated activity.

The invention further provides a kit for determining the presence or absence of human c-MPL. The kit includes: (a) a monoclonal antibody having specific binding activity to c-MPL, or a functional fragment thereof, and (b) a detection reagent. The c-MPL-specific monoclonal antibodies of the kit include monoclonal antibodies having specific binding activity to human c-MPL. Specific examples of anti-human-c-MPL mAbs include 1.6; 1.75; 1.78; 1.111; 1.36; or 1.169; or a monoclonal antibody that can inhibit the binding activity of one or more of these antibodies to c-MPL. The kit of the invention also can include c-MPL polypeptides, one or more ancillary reagents and/or a c-MPL agonist.

As described previously, the monoclonal antibodies of the invention can be used in the methods of the invention to determine the presence or absence of a c-MPL on a cell, tissue or organ. Determination of either the presence or the absence of c-MPL on a cell type or subset of cell types within a tissue can be used, for example, as a marker to diagnose a particular cell types such as hematopoietic progenitor cells or platelets; or tissues, to monitor appearance, growth or progression of c-MPL positive cells. This determination of either the presence or the absence of c-MPL on a cell type can advantageously be used to diagnose and/or distinguish particular disease states. For example, Moliterno et al., describe that expression of the platelet TPO receptor (e.g., c-MPL) as determined by immunoblotting, chemical crosslinking or flow cytometry was markedly reduced or absent in 34 of 34 PV (polycythemia vera) patients and also in 13 of 14 IMF (idiopathic myelofibrosis) patients (Moliterno et al., Stem Cells 1998; 16 (suppl 2):185-192). Accordingly, the markedly reduced presence, or complete absence, of c-MPL on the surface of platelets of particular subjects, as determined using the invention diagnostic methods and the invention anti-c-MPL mAbs provided herein, can be used to identify patients as having PV or IMF; and/or to distinguish PV from other forms of erythrocytosis.

In other embodiments, determination of either the presence or absence of c-MPL on a cell type or subset of cell types within a tissue also can be used, for example, as a marker to monitor the prognosis of a therapeutic treatment such as those directed to stimulating the proliferation, differentiation, migration or survival of hematopoietic cells or tissues, or to determine the applicability of a c-MPL-mediated treatment for a particular disease or pathological condition. Therefore, in accordance with another embodiment, the invention provides diagnostic systems, particularly in the form of a kit, for easy and efficient performance of the methods of the invention.

The kits of the invention for determining the presence or absence of c-MPL on a cell, including human c-MPL expressed on the surface of a human cell, include at least one monoclonal antibody of the invention having c-MPL-specific binding activity, or a functional fragment thereof. A c-MPL-specific monoclonal antibody can be, for example, mAb: 1.6; 1.75; 1.78; 1.111; 1.36; or 1.169, which are specific for human c-MPL. A c-MPL-specific monoclonal antibody included in a kit of the invention also can be a monoclonal antibody that inhibits the binding of one or more of the above monoclonal antibodies to c-MPL. Therefore, one or more of any of the monoclonal antibodies of the invention described herein can be included in a kit of the invention. These c-MPL-specific monoclonal antibodies can be used to contact a sample and determine the specific binding or lack of binding to the sample. Specific binding to the sample indicates the presence of c-MPL in the sample whereas little or no specific binding indicates the absence of c-MPL in the sample.

A kit for determining the presence or absence of c-MPL on a cell can include two or more c-MPL-specific antibodies of the invention. One or more additional monoclonal antibodies can be included to, for example, confirm the binding or non-binding of a first c-MPL-specific monoclonal antibody. Alternatively, the one or more additional monoclonal antibodies can be employed for the testing of additional samples. For example, two c-MPL-specific monoclonal antibodies can be included in a kit of the invention and both monoclonal antibodies can be used to detect the presence or absence of c-MPL in the same sample or each monoclonal antibody can be used to detect the presence or absence of c-MPL in two different samples. Similarly, three or more c-MPL-specific monoclonal antibodies of the invention can be included in a kit of the invention and can be used to detect the presence or absence of c-MPL in the same sample or in two or three different samples. Given the teachings and guidance provided herein, those skilled in the art will understand that a c-MPL-specific monoclonal antibodies of the invention can be used alone or in combination with one or more different c-MPL-specific antibodies of the invention for the purpose of either a primary or confirmatory determination or both.

As with the methods of the invention, the kits for determining the presence or absence of c-MPL in a sample are useful for determining c-MPL on a cell surface or on the cell surface within in a tissue or organ. Similarly, depending on the format chosen, the kits of the invention also can be used to determine the presence or absence of c-MPL within a substructure or component cell type of a tissue or organ. Use of the kits of the invention with a cell lysate can determine the presence or absence of a c-MPL polypeptide expression.

A suitable diagnostic system includes at least one c-MPL-specific monoclonal antibody of the invention, as a separately packaged chemical reagent(s) in an amount sufficient for at least one assay. The kit of the invention can include, for example, various appropriate buffers and solutions for practice of the methods of the invention as described herein. Alternatively, those skilled in the art can readily incorporate a c-MPL-specific monoclonal antibody from a kit of the invention in combination with appropriate buffers and solutions for the practice of the methods of the invention.

A suitable diagnostic system can also include at least one detection reagent. The detection reagent includes a molecule or system of molecules that allow measurement of bound c-MPL-specific monoclonal antibody to a c-MPL polypeptide in a sample. Accordingly, a detection reagent allows for identification of the presence or absence of a c-MPL-specific monoclonal antibody to its target. The detection reagent, or components thereof, can be supplied as a separately packaged chemical reagent(s) in an amount sufficient for at least one assay.

A detection reagent can include, for example, both simple and complex primary and/or secondary binding molecule configurations as well as simple or complex label configurations. For example, a detection reagent can be a label attached to a c-MPL-specific monoclonal antibody of the invention. A kit can include, for example, the monoclonal antibody packaged in a labeled configuration. Alternatively, for example, a kit can contain the label and/or regents for attaching the label to a c-MPL-specific monoclonal antibody.

A detection reagent also can include, for example, a label attached to a secondary binding molecule that is specific for a c-MPL-specific monoclonal antibody. The secondary binding molecule can alternatively be packaged in unlabeled form with or without the inclusion of a label and reagents for attachment. Exemplary secondary binding molecules include, for example, antibodies that recognize the non-antigen bind site of a c-MPL-specific monoclonal antibody, protein A, biotin, avidin and streptaviden. Various other configurations for a detection system or reagent are well known to those skilled in the art and can be included in a kit of the invention.

Similarly, various labels useful in conjunction with a detection reagent also are well known in the art. As described above, such labels can be included in a kit of the invention either attached to the primary c-MPL-specific monoclonal antibody, attached to the secondary binding molecule or packaged as a separate chemical reagent(s) in an amount sufficient for at least one assay. Such labels include, for example, radioisotope, fluorescent or luminescent moiety, enzymatic moiety or secondary component configuration such as a secondary binding molecule labeled with biotin and detectable moiety labeled with avidin or streptaviden. Such detection methods are well known in the art and can be readily included or employed in the kits of the invention given the teachings and guidance provided herein.

A kit of the invention can further include an ancillary reagent. An ancillary reagent of a kit refers to any reagent that aids in the preparation or is useful for performing a c-MPL detection method of the invention and which can be packaged in a kit. Therefore, ancillary reagents can be biological and chemical reagents including, for example, isolated polypeptides which are used as controls or standards, chemical reactants, buffers and the like. Ancillary reagents can similarly be inert materials such as written instructions for performing the methods of the kit. Accordingly, an ancillary reagent can include all reagents, instructions, materials and props that are useful in performing a c-MPL detection method of the invention.

For example, A kit containing a c-MPL-specific monoclonal antibody also can contain a reaction cocktail that provides the proper conditions for performing an assay, for example, an ELISA, RIA, Western blot, immunoprecipitation, immunohistochemistry, fluorescent activated cell sorting (FACS), confocal microscopy or other immunoassay, for determining the binding or non-binding of a c-MPL-specific monoclonal antibody to a c-MPL polypeptide in a sample. A kit further can contain control samples that contain known amounts of a c-MPL polypeptide or c-MPL-specific monoclonal antibody binding epitope and, if desired, a second antibody specific for the c-MPL-specific monoclonal antibody. Accordingly, a kit can contain a control cell line expressing c-MPL on its surface.

The contents of the kit of the invention, for example, one or more c-MPL-specific monoclonal antibodies, are contained in packaging material, particularly to provide a sterile, contaminant-free environment. In addition, the packaging material can contain instructions indicating how the materials within the kit can be employed both to detect the presence or absence of a c-MPL polypeptide or to determine the susceptibility of a cell to c-MPL-mediated proliferation, differentiation, migration or survival. The instructions for use typically include a tangible expression describing the reagent concentration or at least one assay method parameter, such as the relative amounts of reagent and sample to be admixed, maintenance time periods for reagent/sample admixtures, temperature, buffer conditions, and the like. The written instructions can include protocols for performing the c-MPL detection methods of the invention in single or multiple sample format.

Additional components of c-MPL detection reagents that can be included in the diagnostic kits of the invention include ancillary reagents such as binding buffers, dilution buffers and wash buffers. Detectable labels, detectable secondary antibodies or binding molecules and the like can similarly be packaged in the kits for determining the presence or absence and/or for quantitating the amount or concentration of c-MPL present in a sample. Additionally, reactants and solutions for quantitating detectable labels also can be packaged into the c-MPL detection kits of the invention. Such reactants include, for example, any of the various enzyme or their substrates described previously or known to those skilled in the art. Solutions required for detecting labels include, for example, scintillation fluid for radioactive labels.

Concentrations and amounts of the various reactants and ancillary reagents will depend on the intended use and format of the kit. For example, a kit for use in a multiwell ELISA format will contain enough reagents to assay a specified number of samples. Typically, the number will coincide with a multiple of the number of wells in the ELISA plate to be used but can include more or less reactants depending on the needs of a particular assay. Such concentrations and amounts are known to those skilled in the art and can be determined by the individual or entity packaging the reagents into the kits of the invention. Similarly, kits formatted for cell sorting or multiplexing formatting will contain a sufficient number of c-MPL-specific monoclonal antibody, detection reagent and, if included, ancillary reagent to perform a specified number of detections for the multi-sample format.

A kit of the invention can further contain a c-MPL agonist. Inclusion of a c-MPL agonist is useful for detecting the susceptibility of a cell to c-MPL-mediated proliferation, differentiation, migration or survival. The c-MPL agonist can be, for example, TPO or the like. As with the other components or reagents of the kit, a c-MPL agonist can be packaged as a separate chemical reagent(s) in an amount sufficient for at least one assay.

It is understood that modifications which do not substantially affect the activity of the various embodiments of this invention are also included within the definition of the invention provided herein. Accordingly, the following examples are intended to illustrate but not limit the present invention.

### EXAMPLES

### Example 1

### Generation of Mabs Against Human C-Mpl

Recombinant human (rhu) c-Mpl protein, containing the entire 490 amino acid ECD with six histidines added at the C-terminus [rhu-c-Mpl (AA1-490-6H)], was expressed in Chinese hamster ovary (CHO) cells and purified to homogeneity. This material was conjugated to PADRE peptide, emulsified in Freund's complete adjuvant (Pierce), and used to immunize female B6 129 PF1/J mice (Jackson Laboratory). Mice with the highest titers after three rounds of immunization were selected and hybridomas were generated by electrofusion of their splenocytes with mouse myeloma (SP2/0) cells as previously described (Wei et al., Hybridoma (Larchmt) (2006)25:115-124).

### Antibody Screening and Purification

Antibodies secreted into the culture medium of mouse hybridomas were captured in assay plates that were coated with goat anti-mouse IgG Fc and then screened for their ability to bind to biotinylated c-Mpl protein in an enzyme-linked immunosorbent assay (ELISA). Binding Abs were selected for subsequent screening by FMAT (Applied Biosystems, FMAT 8100 HTS) and flow cytometry on c-Mpl positive HEL92.1.7 cells, or parental 32D and HEK293 cells and their transfectants over-expressing human c-Mpl protein. Hybridomas with strong and specific binding in all assays were cloned from single cells and expanded for large-scale antibody production. Antibodies were purified from conditioned media by affinity and ion exchange chromatography to over 95% purity. Antibody isotypes were determined using an IsoStrip Mouse Monoclonal Antibody Isotyping Kit (Roche Applied Sciences #11493027001) following the manufacturer's instructions.

### Measurement of Antibody Affinity

Murine mAbs against a recombinant protein containing the entire extracellular domain of c-Mpl (AA1-490-6H) were generated using conventional immunization and hybridoma technologies. Hybridoma antisera with high binding activities to the immobilized antigen and to c-Mpl-expressing cells were selected. The specificity of these hybridomas was confirmed by their lack of binding to control cells that do not express c-Mpl using both fluorescence-based FMAT and flow cytometric analyses. Six hybridomas with the highest binding activity and specificity were cloned from single cells and their secreted mAbs purified from the culture medium for further characterization.

The affinities of these anti-c-Mpl mAbs to the soluble c-Mpl protein were determined in a solution-based Biacore analysis. Briefly, a fixed amount of rhu-c-Mpl was incubated with various concentrations of anti-c-Mpl mAbs at room temperature for ˜4 hours before being run over the same anti-c-Mpl mAbs immobilized at high density on a CM5 surface. In this assay the binding signal is proportional to the concentration of free rhu-c-Mpl at equilibrium with a given antibody concentration. The dissociation equilibrium constant (KD) was obtained from nonlinear regression of the competition curves using an n-curve one-site homogeneous binding model (KinExATM Pro software).

Four of the mAbs, 1.75, 1.6, 1.111 and 1.78, exhibited double-digit pM affinities (Table 4). Antibody 1.75 exhibited the highest affinity (10 pM) while mAbs 1.36 and 1.169 were the weakest binders (480 pM). The commercially available BAH-1 mAb discussed in more detail below showed no binding in the same assay. Interestingly, mAbs 1.6 and 1.111 competed with mAb 1.75 for binding to soluble c-Mpl whereas mAbs 1.78, 1.36, and 1.169 did not (data not shown). This suggests that mAbs 1.6, 1.111 and 1.75 bind to the same or over-lapping epitope(s) that is different from the epitope (or overlapping epitopes) of the other three mAbs 1.78, 1.36, and 1.169.

### Example 2

### Cloning of the Murine Anti-Hutpor (C-Mpl) Antibody Heavy and Light Chains

The sequences of the murine anti-human TPOR light chain and heavy chain variable regions were obtained by the polymerase chain reaction (PCR) amplification technique known as 5′ RACE (rapid amplification of cDNA ends). Total RNA was isolated from two murine hybridomas expressing human TPOR binding monoclonal antibodies, 1.6.1 and 1.75.1, using TRIzol reagent (Invitrogen) followed by a further purification using the RNeasy Mini Kit (Qiagen). Mixed random and oligo-dT primed first strand, RACE ready cDNAs were prepared using the GeneRacer Kit (Invitrogen). PCR amplifications of the cDNAs were performed with Phusion HF DNA polymerase (Finnzymes) with the forward primer, GeneRacer® nested primer (5′-GGA CAC TGA CAT GGA CTG AAG GAG TA-3′) and the reverse primer, 5′-CTC ACA GGT ATA GCT GTT ATG TCG-3′, for the light chain, and 5′-CCT TGR KCT TTG GRG GGA AGA TGA AGA C-3′, for the heavy chain. The PCR reaction cycles consisted of a two minutes denaturation of the cDNA at 94° C., followed by three cycles of amplification with each cycles consisting of 20 seconds at 94° C.; 30 seconds at 55° C.; and 30 seconds at 72° C. plus an additional 27 cycles consisting of 20 seconds at 94° C.; 30 seconds at 60° C.; and 30 seconds at 72° C. The reactions were then incubated for 7 minutes at 72° C. following the last PCR cycle to insure complete elongation. The RACE PCR products were cloned into pCR4-TOPO (Invitrogen) and their sequences determined using ABI DNA sequencing instruments (Perkin Elmer). Consensus sequences were determined using Vector NTI Advance 10 software (Invitrogen) and used to design primers for full-length antibody chain PCR amplification.

To obtain the complete coding region sequences for the expression of anti-huTPOR antibody 1.75.1, PCR was again used. The light chain 5′ PCR primer encoded the amino terminus of the signal sequence, a SalI restriction enzyme site, and an optimized Kozak sequence was (5′-AAG CTC GAG GTC GAC TAG ACC ACC ATG GAT TCT CAG GTG CAG ATT TTC-3′) and the 3′ primer encoded the carboxyl terminus and termination codon, as well as a NotI restriction site was (5′-AAC CGT TTA AAC GCG GCC GCT CAA CAC TCA TTC CTG TTG AAG C-3′). The heavy chain 5′ PCR primer encoded the amino terminus of the signal sequence, a SalI restriction enzyme site, and an optimized Kozak sequence was (5′-AAG CTC GAG GTC GAC TAG ACC ACC ATG ACA TTG AAC ATG CTG TTG-3′) and the 3′ primer encoded the carboxyl terminus and termination codon, as well as a NotI restriction site was (5′-AAC CGT TTA AAC GCG GCC GCT CAT TTA CCC GGA GAC CGG GAG ATG GT-3′). The PCRs were performed using Phusion HF DNA polymerase and the reaction cycles consisted of a two minutes denaturation of the cDNA at 94° C., followed by three cycles of amplification with each cycles consisting of 20 seconds at 94° C.; 30 seconds at 55° C.; and 30 seconds at 72° C. plus an additional 27 cycles consisting of 20 seconds at 94° C.; 30 seconds at 60° C.; and 30 seconds at 72° C. The reactions were then incubated for 7 minutes at 72° C. following the last PCR cycle to insure complete elongation. The resulting PCR products were gel isolated, purified using QIAquick spin columns (Qiagen), digested with SalI (NEBL) and NotI (NEBL), gel isolated and purified using QIAquick spin columns, and then ligated into the mammalian expression vector pTT5.

| MURINE ANTI-HUTPOR VARIABLE REGION NUCLEOTIDE AND AMINO ACID | |
|---|---|
| SEQUENCES | |
| Ab 1.6.1 light chain (SEQ ID NOs: 37 & 38): | |
| | D I Q M T Q S S S S F S V S L G D R V T |
| 1 | GACATCCAGATGACACAATCTTCATCCTCCTTTTCTGTATCTCTAGGAGACAGAGTCACC |
| | I T C M A S E D I Y I R L A W Y Q Q K P |
| 61 | ATTACTTGCATGGCAAGTGAGGACATTTATATTCGCTTAGCCTGGTATCAGCAGAAACCA |
| | G N A P R L L I S A A T S L E T G V P S |
| 121 | GGAAATGCTCCTAGGCTCTTAATATCTGCTGCAACCAGTTTGGAAACTGGGGTTCCTTCT |
| | R F S G S G S G K D Y T L S I T S L Q T |
| 181 | AGATTCAGTGGCAGTGGATCTGGAAAGGATTACACTCTCAGCATTACCAGTCTACAGACT |
| | E D V G T Y Y C Q Q Y W S S P W T F G G |
| 241 | GAAGATGTTGGTACATATTACTGTCAACAGTATTGGAGTTCTCCGTGGACGTTCGGTGGA |
| | G T K L E I N |
| 301 | GGCACCAAGCTGGAAATCAAT |
| Ab 1.6.1 heavy chain (SEQ ID NOs: 39 & 40): | |
| | D V Q L Q E S G P G L V K P S Q S L S L |
| 1 | GATGTGCAGCTTCAGGAGTCGGGACCTGGCCTGGTGAAACCTTCTCAGTCTCTGTCCCTC |
| | T C T V T G Y S I T S D Y T W N W I R Q |
| 61 | ACCTGCACTGTCACTGGCTACTCAATCACCAGTGATTATACCTGGAACTGGATCCGGCAG |
| | F P G N K L E W M G Y I T Y S G S T S Y |
| 121 | TTTCCAGGAAACAAACTGGAGTGGATGGGCTACATAACTTACAGTGGTAGCACTAGCTAC |
| | N P S L K S R N S I T R D T S K N Q F F |
| 181 | AACCCATCTCTCAAAAGTCGAAACTCTATCACTCGAGACACATCCAAGAACCAGTTCTTC |
| | L Q L N S V T T E D T A T Y Y C A R L G |
| 241 | CTGCAGTTGAATTCTGTGACTACTGAGGACACAGCCACATATTACTGTGCAAGACTGGGA |
| | R R Y T M D Y W G Q G T S V T V S S |
| 301 | CGTCGCTATACTATGGACTACTGGGGTCAAGGAACCTCAGTCACCGTCTCCTCA |
| Ab 1.75.1 light chain (SEQ ID NOs: 41 & 42): | |
| | Q I V L T Q S P A I M S A S P G E K V T |
| 1 | CAAATTGTTCTCACCCAGTCTCCAGCAATCATGTCTGCATCTCCAGGGGAGAAGGTCACC |
| | I S C S A S S S V S Y M Y W Y Q Q K P G |
| 61 | ATATCCTGCAGTGCCAGCTCAAGTGTAAGTTACATGTACTGGTACCAGCAGAAGCCAGGA |
| | S S P K P W I Y R T S N L A S G V P A R |
| 121 | TCCTCCCCCAAACCCTGGATTTATCGCACATCCAACCTGGCTTCTGGAGTCCCTGCTCGC |
| | F S G S G S G T S Y S L T I S N M E A E |
| 181 | TTCAGTGGCAGTGGGTCTGGGACCTCTTACTCTCTCACAATCAGCAACATGGAGGCTGAA |
| | D A A A Y Y C Q Q Y H S Y P T T F G G G |
| 241 | GATGCTGCCGCTTATTACTGCCAGCAGTATCATAGTTACCCAACCACGTTCGGAGGGGGG |
| | T K L E V K |
| 301 | ACCAAGCTGGAAGTGAAA |
| Ab 1.75.1 heavy chain (SEQ ID NOs: 43 & 44): | |
| | E V Q L V E S G G G L V Q P K G S L K L |
| 1 | GAGGTGCAGCTTGTTGAGTCTGGTGGAGGATTGGTGCAGCCTAAAGGGTCATTGAAACTC |
| | S C A A S G F S F N T Y A M N W V R Q A |
| 61 | TCATGTGCAGCCTCTGGATTCAGCTTCAATACCTACGCCATGAACTGGGTCCGCCAGGCT |
| | P G K G L E W I A H I R S K S N N F A T |
| 121 | CCAGGAAAGGGTTTGGAATGGATTGCTCACATAAGAAGTAAAAGTAATAATTTTGCAACA |
| | Y Y A D S V K D R F S I S R D A S E N I |
| 181 | TATTATGCCGATTCAGTGAAAGACAGATTCAGCATCTCCAGAGATGCTTCAGAAAACATT |
| | L F L Q M N N L K T E D T A M Y Y C V R |
| 241 | CTCTTTCTGCAAATGAACAACTTGAAAACTGAGGACACAGCCATGTATTATTGTGTGAGA |
| | Q G G D F P M D Y W G Q G T S V T V S S |
| 301 | CAAGGGGGTGACTTTCCTATGGACTACTGGGGTCAAGGAACCTCAGTCACCGTCTCCTCA |
| Ab 1.78.1 light chain (SEQ ID NOs: 45 & 46): | |
| | D I V M T Q A A P S I P V T P G E S V S |
| 1 | GATATTGTGATGACTCAGGCTGCACCCTCTATTCCTGTCACTCCTGGAGAGTCAGTATCC |
| | I S C R S D K S L L H S N G N T Y L F W |
| 61 | ATCTCCTGCAGGTCTGATAAGAGTCTCCTGCATAGTAATGGCAACACTTACTTGTTTTGG |
| | F L Q R P G Q S P Q L L I Y R M S N L A |
| 121 | TTCCTGCAGAGGCCAGGCCAGTCTCCTCAGCTCCTGATATATCGGATGTCCAACCTTGCC |
| | S G V P D R F S G S G S G T A F T L R I |
| 181 | TCAGGAGTCCCAGACAGGTTCAGTGGCAGTGGGTCAGGAACTGCTTTCACACTGAGAATC |
| | S G V E A E D V G V Y Y C M Q H L E Y P |
| 241 | AGTGGAGTGGAGGCTGAGGATGTGGGTGTTTATTACTGTATGCAACATCTAGAATATCCG |
| | Y T F G G G T K L E I K |
| 301 | TACACGTTCGGAGGGGGGACCAAGCTGGAAATAAAA |
| Ab 1.78.1 heavy chain (SEQ ID NOs: 47 & 48): | |
| | Q V Q L Q Q S G P E L V K P G A S V K M |
| 1 | CAGGTTCAACTGCAGCAGTCTGGACCTGAACTGGTGAAGCCTGGGGCCTCAGTGAAGATG |
| | S C K A S G Y A F S S S W L N W V R Q R |
| 61 | TCCTGCAAGGCTTCTGGCTACGCATTCAGTAGTTCCTGGTTGAACTGGGTGAGGCAGAGG |
| | P G K G L E W I G R I Y P G D G E N H Y |
| 121 | CCTGGAAAGGGTCTTGAGTGGATTGGACGGATTTATCCTGGAGATGGAGAAAATCACTAT |
| | N G K F K G K A T L T A D K S S S T G Y |
| 181 | AATGGGAAATTCAAGGGCAAGGCCACACTGACTGCAGACAAATCCTCCAGCACAGGCTAC |
| | M Q L S S L T S E D S A V Y F C A S Y Y |
| 241 | ATGCAACTCAGCAGCCTGACGTCTGAGGACTCTGCGGTCTACTTCTGTGCAAGTTATTAT |
| | E G G Y W G Q G T L I T V S A |
| 301 | GAAGGGGGTTATTGGGGCCAAGGGACTCTAATCACTGTCTCTGCA |
| Ab 1.111.1 light chain (SEQ ID NOs: 86 & 87): | |
| | D I Q M T Q S S S S F S V S L G D R V T |
| 1 | GACATCCAGATGACACAATCTTCATCCTCCTTTTCTGTGTCTCTAGGAGACAGAGTCACC |
| | I T C K A S E D I Y I R L A W Y Q Q K P |
| 61 | ATTACTTGCAAGGCAAGTGAGGACATATATATTCGCTTAGCCTGGTATCAGCAGAAACCA |
| | G N A P R L L I S A A T S L E T G I P S |
| 121 | GGAAATGCTCCTAGGCTCTTAATATCTGCTGCAACCAGTTTGGAAACTGGGATTCCTTCA |
| | R F S G S G S G E D Y T L T I T S L Q T |
| 181 | AGATTCAGTGGCAGTGGATCTGGAGAGGATTACACTCTCACCATTACCAGTCTTCAGACT |
| | E D V A T Y Y C Q Q Y W T T P W T F G G |
| 241 | GAAGATGTTGCTACTTATTACTGTCAACAATATTGGACTACTCCGTGGACGTTCGGTGGA |
| | G T K L E I K R |
| 301 | GGCACCAAGCTGGAAATCAAACGG |
| Ab 1.111.1 heavy chain (SEQ ID NOs: 88 & 89): | |
| | D V Q L Q E S G P G L V K P S Q S L S L |
| 1 | GATGTGCAACTTCAGGAGTCGGGACCTGGCCTGGTGAAACCTTCTCAGTCTCTGTCCCTC |
| | T C T V T G Y S I T I D Y T W N W I R Q |
| 61 | ACCTGCACTGTCACTGGCTACTCAATCACCATTGATTATACCTGGAACTGGATCCGGCAG |
| | F P G N K L E W M G Y I T Y S G S T D Y |
| 121 | TTTCCAGGAAACAAACTGGAGTGGATGGGCTATATAACGTACAGTGGTAGCACTGACTAC |
| | N P S L K S R S S I T R D T S M N Q F F |
| 181 | AACCCATCTCTCAAAAGTCGAAGCTCTATCACTCGAGACACATCCATGAACCAGTTCTTC |
| | L Q L N S V T T E D T A T Y Y C A R L G |
| 241 | CTGCAATTGAATTCTGTGACTACTGAGGACACAGCCACATATTACTGTGCAAGACTGGGA |
| | R R Y A L D Y W G Q G T S V T V S S |
| 301 | CGTCGCTATGCTTTGGACTATTGGGGTCAAGGAACCTCAGTCACCGTCTCCTCA |
| Ab 1.36.1 light chain (SEQ ID NOs: 90 & 91): | |
| | D I V M T Q A A P S V P V T P G E S V S |
| 1 | GATATTGTGATGACTCAGGCTGCACCCTCTGTACCTGTCACTCCTGGAGAGTCAGTATCC |
| | I S C R S S K S L L H S N G N T Y L Y W |
| 61 | ATCTCCTGCAGGTCTAGTAAGAGTCTCCTGCATAGTAATGGCAACACTTACTTGTATTGG |
| | F L Q R P G Q S P Q L L I Y R M S N L A |
| 121 | TTCCTGCAGAGGCCAGGCCAGTCTCCTCAACTCCTGATATATCGGATGTCCAACCTTGCC |
| | S G V P D R F S G S G S G T A F T L R I |
| 181 | TCAGGAGTCCCAGACAGGTTCAGTGGCAGTGGGTCAGGAACTGCTTTCACACTGAGAATC |
| | S R V E A E D V G V Y Y C M Q H L E Y P |
| 241 | AGTAGAGTGGAGGCTGAGGATGTGGGTGTTTATTACTGTATGCAACATCTAGAATATCCG |
| | Y T F G G G T K L E I K R |
| 301 | TACACGTTCGGAGGGGGGACCAAGCTGGAAATAAAACGG |
| Ab 1.36.1 heavy chain (SEQ ID NOs: 92 & 93): | |
| | Q V Q L Q Q S G P E L V K P G A S V K I |
| 1 | CAGGTTCAGCTACAGCAGTCTGGACCTGAGCTGGTGAAGCCTGGGGCCTCAGTGAAGATT |
| | S C K A S G Y G F S N S W M N W V R Q R |
| 61 | TCCTGCAAGGCTTCTGGCTACGGATTCAGTAACTCCTGGATGAACTGGGTGAGGCAGAGG |
| | P G K G L E W I G R I Y P G D G E T S Y |
| 121 | CCTGGAAAGGGTCTTGAGTGGATTGGACGGATTTATCCTGGAGATGGAGAGACTAGCTAC |
| | N G E F V G K A T L T A D K S S S T A Y |
| 181 | AATGGGGAGTTCGTGGGCAAGGCCACACTGACTGCAGACAAATCTTCCAGCACAGCCTAC |
| | M H L S S L T S E D S A V Y F C A S Y Y |
| 241 | ATGCACCTCAGCAGCCTGACATCTGAGGACTCTGCGGTCTACTTCTGTGCAAGCTACTAT |
| | E G G Y W G Q G T L V T V S |
| 301 | GAAGGGGGTTACTGGGGCCAAGGGACTCTGGTCACTGTCTCT |
| Ab 1.169.1 light chain (SEQ ID NOs: 94 & 95): | |
| | D I V M T Q A A P S L P V T P G E S V S |
| 1 | GATATTGTGATGACTCAGGCTGCACCCTCTCTTCCTGTCACTCCTGGAGAGTCAGTATCC |
| | I S C R S S K S L L H S N G N T Y L F W |
| 61 | ATCTCCTGCAGGTCTAGTAAGAGTCTCCTGCATAGTAATGGCAACACTTACTTGTTTTGG |
| | F L Q R P G Q S P H L L I Y R M S N L A |
| 121 | TTCCTGCAGAGGCCAGGCCAGTCTCCTCACCTCCTGATATATCGGATGTCCAACCTTGCC |
| | S G V P D R F S G S G S G T A F T L R I |
| 181 | TCAGGAGTCCCAGACAGGTTCAGTGGCAGTGGGTCAGGAACTGCTTTCACACTGAGAATC |
| | S R V E A E D V G V Y Y C M Q H L E Y P |
| 241 | AGTAGAGTGGAGGCTGAGGATGTGGGTGTTTATTACTGTATGCAACATCTAGAATATCCG |
| | Y T F G G G T K L E I K R |
| 301 | TACACGTTCGGAGGGGGGACCAAGCTGGAAATAAAACGG |
| Ab 1.169.1 heavy chain (SEQ ID NOs: 96 & 97): | |
| | Q V Q L Q Q S G P E L V K P G A S V K I |
| 1 | CAGGTTCAGCTGCAGCAGTCTGGACCTGAGCTGGTGAAGCCTGGGGCCTCAGTGAAGATT |
| | S C K A S G Y G F S S S W M N W V K Q R |
| 61 | TCCTGCAAGGCTTCTGGCTACGGATTCAGTAGCTCCTGGATGAACTGGGTGAAGCAGAGG |
| | P G K G L E W I G R I Y P G D G E T S Y |
| 121 | CCTGGAAAGGGTCTTGAGTGGATTGGACGGATTTATCCTGGAGATGGAGAGACTAGCTAC |
| | N G E F K G K A T L T A D K S S S T A Y |
| 181 | AATGGGGAGTTCAAGGGCAAGGCCACACTGACTGCCGACAAATCCTCCAGCACAGCCTAC |
| | M Q L S S L T S E D S A V Y F C A S Y Y |
| 241 | ATGCAACTCAGCAGCCTGACATCTGAGGACTCTGCGGTCTACTTCTGCGCAAGCTACTAT |
| | E G G Y W G Q G T L V T V S A |
| 301 | GAAGGGGGTTACTGGGGCCAAGGGACTCTGGTCACTGTCTCTGCA |

### Example 3

### Flow Cytometric Analysis

### Cell Lines

Human B cell leukemia lines BV-173 and EHEB, human acute megakaryocytic leukemia lines CMK and Mole, and the human Hodgkin's lymphoma line L428 were purchased from DSMZ GmbH (The German Research Centre for Biological Material, Braunschweig, Germany). All other cell lines were purchased from the American Type Culture Collection (ATCC). Cell lines were cultured according to supplier's instructions with TF-1 cells cultured in the presence of GM-CSF. 32D-hu-c-Mpl and Ba/F3-hu-c-Mpl cells that stably express the full-length human c-Mpl gene have been described previously (Bartley et al., Cell (1994)77:1117-1124; de Sauvage et al., Nature (1994)369:533-538). HEK293-hu-c-Mpl cells were generated by transiently transfecting HEK293 cells with an expression vector encoding full-length human c-Mpl with a C-terminal flag tag, and used 48 hr later.

### Flow Cytometry

Flow cytometry was performed on cell lines in log-phase growth. Adherent cells were harvested from tissue culture plates using EDTA dissociation buffer to avoid destruction of cell surface proteins and washed in phosphate-buffered saline (PBS) before being resuspended in FACS buffer [PBS plus 2% fetal bovine serum (FBS)]. Cells or platelets (0.2−1×10⁶ in 200 μl of FACS buffer) were incubated with 1 μg/ml of primary Abs or the appropriate isotype control Abs at 4° C. for 1 hr, washed three times with FACS buffer, then incubated with 1 μg/ml of secondary Ab at 4° C. for 30 min. Eight commercially available anti-c-Mpl Abs were evaluated: BAH-1 (mAb, IgG1) from BD Biosciences, mAbs 1016 (IgG2a) and 10161 (IgG2b) and goat polyclonal antibody (pAb) AF1016 from R&D Systems, c-Mpl-JJ-12 (mAb, IgG2b), c-Mpl-N-20 (goat pAb) and c-Mpl-H-300 (rabbit pAb) from Santa Cruz Biotechnology, Inc. (SC), and a rabbit pAb c-Mpl 2474 from Strategic Diagnostics, Inc. (SDI). Anti-CD34-PE and anti-CD61-FITC mAbs were from BD Biosciences. Alexa Fluor 647-conjugated anti-c-Mpl mAbs 1.6 or 1.75 were prepared in our laboratories using standard Ab conjugation chemistry. After staining, cells were washed, resuspended in FACS buffer containing 7-AAD (omitted for platelets), and analyzed using a FACS Calibur flow cytometer with FCS Express software (BD Biosciences). For soluble receptor competition experiments, anti-c-Mpl mAbs were preincubated with an excess of soluble c-Mpl or EpoR protein at RT for 30 min. This mixture was then used to stain cells or platelets as described above.

### Invention Anti-C-Mpl Mabs Bind Specifically to Cells Engineered to Express Human C-Mpl

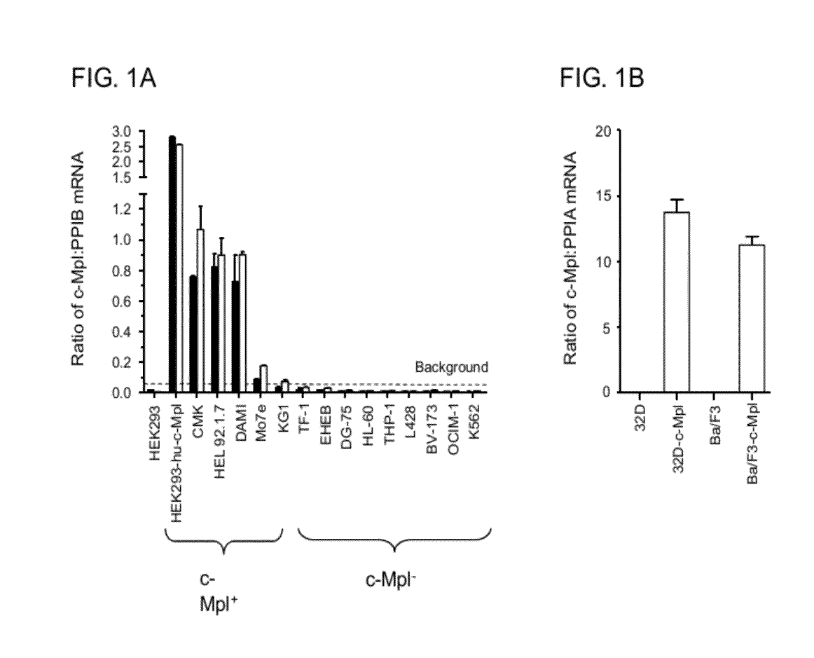

To evaluate the binding of these Abs to c-Mpl expressed on the surface of intact cells we conducted flow cytometry experiments using three different cell lines engineered to express high levels of human c-Mpl. Human HEK293-hu-c-Mpl cells were generated by transiently transfecting a full-length human c-Mpl expression construct into parental HEK293 cells. The transfected cells expressed at least 2.5× more human c-Mpl mRNA than was found in various hematopoietic cell lines (FIG. 1A). Two murine cells lines, Ba/F3 and 32D, were stably transfected with the human c-Mpl gene. While the non-transfected parental cells showed no detectable c-Mpl mRNA, Ba/F3-hu-c-Mpl and 32D-hu-c-Mpl cell lines expressed significant levels of c-Mpl mRNA (FIG. 1B) and are routinely used in cell-based functional assays to evaluate the biological activities of rhuTPO and TPO mimetics.

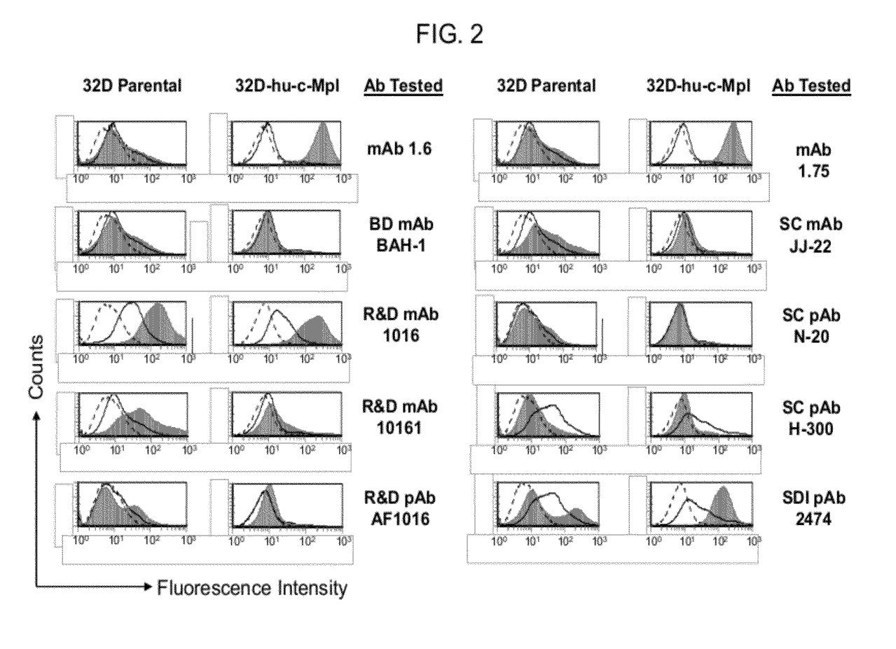

As shown in FIG. 2, mAbs 1.6 and 1.75 bound to virtually all 32D-hu-c-Mpl cells resulting in a complete shift of the fluorescence histogram compared to that of unstained cells or those stained with isotype control Ab. No binding was seen on parental 32D cells, suggesting specific binding of these mAbs to human c-Mpl. Similar results were seen with the four other mAbs 1.111, 1.169, 1.36 and 1.78 set for the in Table 4, though mAbs 1.6 and 1.75 gave the greatest shift in mean fluorescence intensity when equal concentrations of Abs were used, consistent with their higher affinities.

Parental control or transfected 32D cells were also stained with eight commercially available anti-c-Mpl Abs that were raised against various parts of the N-terminal domain of human c-Mpl or intact CMK cells, a megakaryocytic cell line. All of these Abs either failed to show any binding to 32D-hu-c-Mpl cells or bound at similar levels to both transfected and untransfected 32D cells, suggesting that they do not bind specifically to human c-Mpl (FIG. 2). Similar results were obtained when Ba/F3-hu-c-Mpl and HEK293-hu-c-Mpl cells with their corresponding non-transfected parental controls were used.

### Anti-C-Mpl Mabs Bind to Human Megakaryocytic Cell Lines Expressing Endogenous C-Mpl

We compared the staining patterns of our anti-c-Mpl mAbs to commercially available Abs on human hematopoietic cell lines that express endogenous c-Mpl.

Branched DNA analysis, a sensitive and high-throughput method for RNA quantitation in cells, was used to identify cell lines that express human c-Mpl mRNA. Expression of c-Mpl mRNA in cell lysates was measured using the QuantiGene® branched DNA (bDNA) assay (Affymetrix, Foster City, Calif.) following the manufacturer's instructions and the QG2.0 reagent system. The human c-Mpl bDNA probe sets used detect sequences spanning nucleotides 63-496 and 1598-1899 of GenBank REFSEQ NM_{—}005373 that corresponds to the extracellular and intracellular portions of the protein, respectively. c-Mpl mRNA expression levels were normalized to that of the housekeeping genes cyclophilin B (PPIB; NM_{—}000942) or cyclophilin A (PPIA, NM_{—}008907) in human and murine cells, respectively. All assays were run in triplicate.

Consistent with published findings, the megakaryocytic leukemia cell lines CMK, DAMI and Mo7e, and an erythroid leukemia cell line HEL 92.1.7 were found to express c-Mpl mRNA among 14 cell lines tested (FIG. 1A). While similar c-Mpl mRNA levels were seen in CMK, DAMI and HEL 92.1.7 cells, Mo7e cells expressed an ˜10-fold lower level of c-Mpl mRNA. c-Mpl mRNA was not detected above background levels in nine other cell lines tested, including two erythroid leukemia cell lines (OCMI-1, and granulocyte macrophage colony-stimulating factor [GM-CSF]-dependent TF-1 cells), four lymphocytic cell lines (BV-173, DG-75, EHEB, L428) and three myeloid or monocytic cell lines (K562, HL-60, THP-1). A very low level of c-Mpl mRNA was detected in the acute myeloid leukemia cell line, KG-1 (FIG. 1A).

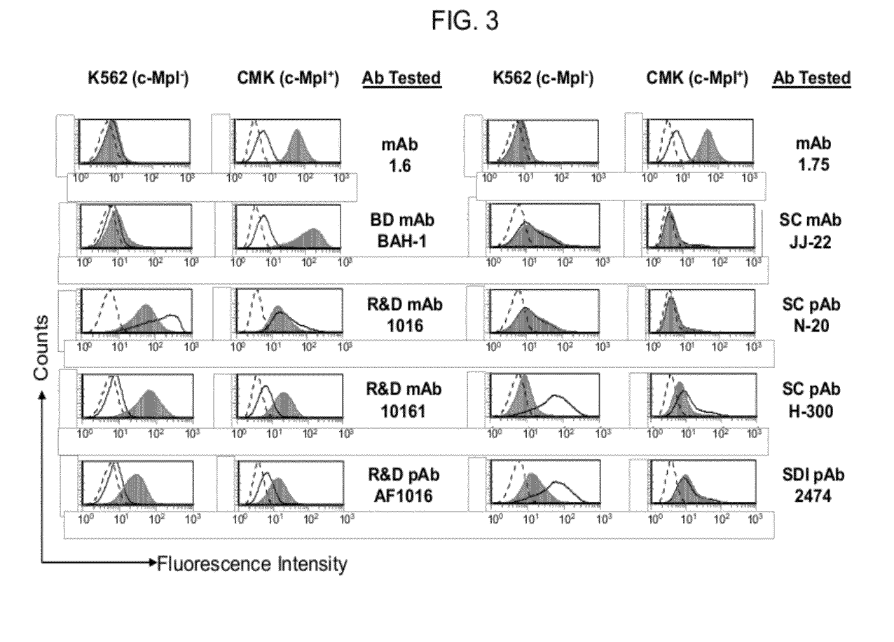

Consistent with previous results using engineered cells, mAbs 1.6 and 1.75 bound CMK cells, a c-Mpl mRNA positive line, without detectable binding to K562 cells, a c-Mpl mRNA negative line (FIG. 3). In contrast, with the exception of the BAH-1 mAb, all of the commercially available Abs bound CMK and K562 cells to similar degrees, and in several cases the Ab actually stained K562 cells more strongly than CMK cells, suggesting that staining was non-specific. The BAH-1 mAb was unique among the commercial reagents in that it stained CMK cells intensely. However, it also bound K562 cells weakly with ˜10% of cells shifted relative to the staining observed with the isotype control Ab (FIG. 3).

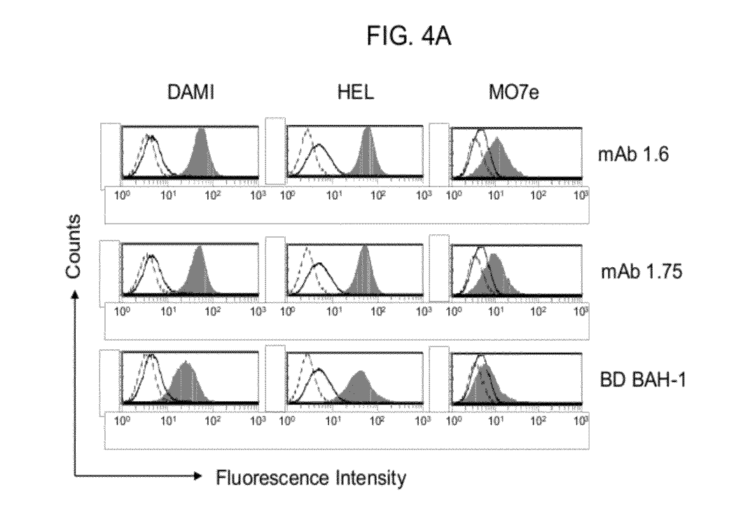

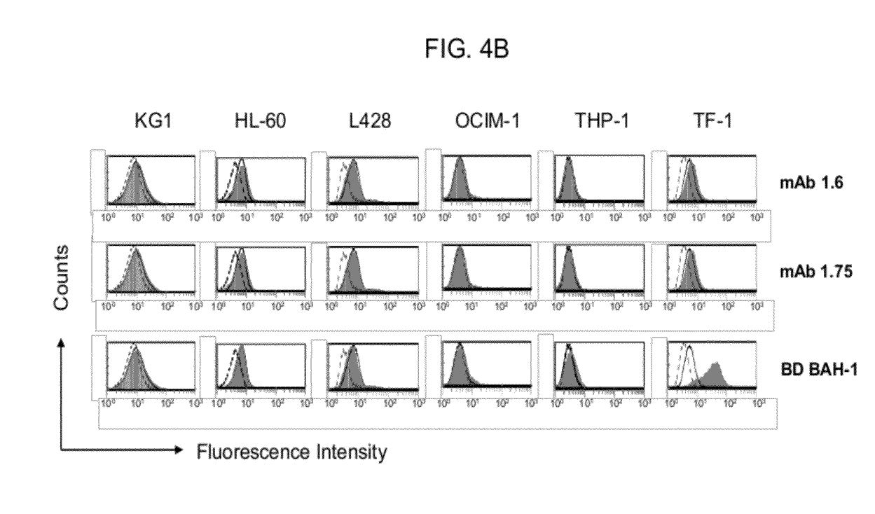

Monoclonal Abs 1.6, 1.75 and BAH-1 were selected for further characterization. We expanded the flow cytometry analysis to 9 additional hematopoietic cell lines, three of which expressed c-Mpl mRNA at high levels (DAMI, HEL and Mo7e), KG-1 cells that expressed marginal levels of c-Mpl mRNA, and five lines that did not express detectable levels of c-Mpl mRNA (HL-60, L428, OCIM-1, THP-1 and TF-1). As shown in FIG. 4, mAbs 1.6 and 1.75 consistently stained all of the strongly c-Mpl+ cell lines but not KG-1 or any of the c-Mpl-cell lines. The shift of the fluorescence profiles following staining with mAbs 1.6 and 1.75 correlated with mRNA levels in the positive cell lines, with staining of Mo7e cells being ˜10-fold lower than that of CMK, DAMI, and HEL cells (compare FIGS. 1 and 4A). The BAH-1 mAb stained all of the c-Mpl+ cells, again at levels that correlated with mRNA expression levels, though CMK cells appeared to stain disproportionately more brightly. However, BAH-1 also stained TF-1 cells which, like K562 cells shown above, also do not express c-Mpl mRNA (compare FIGS. 1 and 4B).

### Anti-C-Mpl Mabs Bind to Purified Primary Human Platelets and CD34+ Hematopoietic Cells

Whole blood was collected by venipuncture from forearms of healthy adult volunteers who self reported to be drug- and supplement-free for at least 10 days prior to donation. After discarding the first 2 ml of blood, 40 ml of blood was collected into a syringe preloaded with one part of ACD buffer (150 mM sodium citrate, 120 mM citric acid, 250 mM glucose, pH 6.5) to nine parts of whole blood. Samples were centrifuged at 200×g for 15 min at room temperature and platelet-rich-plasma (PRP) was pipetted off the top layer. The PRP was centrifuged again at 540×g for 15 min and the platelet pellet was resuspended in 15 ml of Tyrode's solution (137 mM NaCl, 12 mM NaHCO3, 5.5 mM glucose, 2 mM KCl, 1 mM MgCl2, and 0.3 mM Na2HPO4, pH 7.4). Freshly isolated platelets were found to be stable in Tyrode's solution for up to one week, which could be extended to one month if fixed with 2% paraformaldehyde.

Primary human CD34+ cells isolated from normal bone marrow or G-CSF-mobilized peripheral blood were purchased from AllCells Inc. (Emeryville, Calif.). Cryopreserved cells were thawed and cultured overnight in StemPro-34 medium (GIBCO) containing StemPro nutrient supplement, 2.5 μg/ml fungizone, 1× penicillin-streptomycin-glutamine, and 100 ng/ml recombinant human stem cell factor (Amgen).

Human platelets express c-Mpl on the surface (Fielder et al., Blood (1996)87:2154-2161; Li et al., Br J Haematol (1999)106:345-356). To evaluate whether our new mAbs could be used to detect c-Mpl on these blood elements, platelets from healthy donors were isolated and stained with mAbs 1.6, 1.75 and BAH-1 and analyzed by flow cytometry. CD61, an abundant platelet-specific cell surface glycoprotein, was expressed at high levels on these platelet preparations, confirming their purity. Similarly, mAbs 1.6 and 1.75 bound to human platelets in a dose-dependent manner, with detectable staining observed at a concentration of >0.01 μg/ml. The mean fluorescence intensity increased with increasing concentrations of mAbs and plateaued at ˜1 μg/ml. In contrast, the BAH-1 mAb did not bind to platelets at any of the Ab concentrations tested up to 5 μg/ml.

Genetic and biochemical studies suggest that primitive human CD34+ hematopoietic stem cell and/or progenitor cells also express c-Mpl. To study c-Mpl expression on these cells, mAbs 1.6 and 1.75 were conjugated with the fluorescent dye Alexa Fluor 647 and used to stain human CD34+ cells that had been purified from bone marrow (BM) or G-CSF-mobilized peripheral blood mononuclear cells (PBMC). Purified CD34⁺ cells were labeled with anti-CD34˜PE, anti-CD61˜FITC, and mAb 1.6˜Alexa647 or their respective fluorochrome-conjugated isotype control Abs. In representative examples, approximately 20% of BM-derived CD34+ and 39% of PBMC-derived CD34+ cells expressed human c-Mpl protein on their surface. The majority of cells in this population, approximately 17% or 36% of the total population, were c-Mpl+CD61− while approximately 3% of the total population were either c-Mpl+CD61+ or c-Mpl−CD61+. CD34+ cells from four different BM donors and six different PBMC donors were tested with a range of 10-20% and 20-50% c-Mpl+ cells observed, respectively.

### Example 4

### Receptor Competition and siRNA Knock Down Assays Soluble Receptor Competition Studies

To further confirm the specificity of mAbs 1.6 and 1.75, we performed receptor competition and siRNA knock-down studies using HEL 92.1.7 cells. In the competition studies, increasing molar ratios (e.g 1, 0; 3:1 and 30:1) of the soluble extracellular domains of rhu-c-Mpl (AA1-490-6H) or EpoR (EpoR-ECD) were preincubated with the invention anti-c-Mpl mAbs 1.6 and 1.75 prior to their use to stain HEL 92.1.7 cells; along with prior art antibody BAH-1. The results indicate that soluble c-Mpl receptor efficiently blocked the ability of mAb 1.6 to bind to HEL 92.1.7 cells, while EpoR-ECD had no effect even when used at a 30 to 1 molar excess. This demonstrates that mAb 1.6 binds specifically to human c-Mpl. On the other hand, neither soluble c-Mpl nor EpoR-ECD were able to block BAH-1 mAb binding, indicating that this Ab does not bind to either receptor. Similar results were seen when mAb 1.75 was used in the same experiment and when binding was evaluated on CMK cells or human platelets. The inability of the BAH-1 mAb to bind to soluble c-Mpl protein was also confirmed by Biacore analysis and ELISA studies.

siRNA Knock Down Studies

Small inhibitory (si)RNAs with the following c-Mpl target sequences were purchased from Qiagen (Valencia, Calif.; GeneSolution siRNAs,) and Invitrogen (Carlsbad, Calif.; BLOCK-iT™ RNAi Designer,): c-Mpl #1,5′-GCTCCCAAGGCTTCTTCTA-3′; c-Mpl #2,5′-ACGAGGATTGAGATAATCTAA-3′; and c-Mpl #3,5′-CCAGTGGGCATCTGGAATT-3′. Non-targeting siRNAs were 5′-ACGTACGCGGAATACTTCGTT-3′ (anti-Luciferase), 5′-AATTCTCCGAACGTGTCACGT-3′ (Qiagen, Negative Control siRNA #1027310) and AllStars Negative Control (proprietary sequence, Qiagen, #1027280). HEL92.1.7 cells (5×106) were transfected in suspension with 400 nM siRNAs. The next day, c-Mpl gene silencing was evaluated in an aliquot of cells by branched DNA assay and the remainder of the cells was cultured for 48 or 72 hours post-transfection prior to flow cytometric analysis for c-Mpl protein. Cells were analyzed by flow cytometry and the level of fluorescence compared to that of cells incubated with isotype control primary Ab plus PE-conjugated secondary Ab.

Three human c-Mpl-specific siRNAs were used to knock down human c-Mpl mRNA expression by at least 60% in transiently transfected HEL 92.1.7 cells. Two or three days later, knocked down cells were stained with mAbs 1.6, 1.75 or BAH-1 and analyzed by flow cytometry. Compared to cells transfected with control siRNAs which minimally affected the binding of these mAbs, all three c-Mpl siRNAs reduced the binding of mAbs 1.6 and 1.75 whereas binding of BAH-1 was not affected. These experiments further demonstrate that mAbs 1.6 and 1.75 bind to human c-Mpl specifically while the BAH-1 mAb is not specific for c-Mpl.

### Example 5

### Detecting the Neutralizing Activities of the Invention Anti-C-Mpl Mabs

32D-hu-c-Mpl cells were maintained in MEM (Invitrogen) containing 10% fetal bovine serum and 1 ng/ml of mouse Interleukin-3 (mIL-3). On the day of the assay, cells in mid-log phase growth were washed in MEM growth medium to remove mIL-3 before seeded in 96-well cell culture plates in triplicates at 1×10⁴ cells/well in 50 μL, MEM.

To study the agonistic activity of the 6 anti-hu-c-Mpl mAbs set forth in Table 4 (e.g., mAbs: 1.75; 1.6; 1.111; 1.78; 1.36; and 1.169), 9 different concentrations of each mAb, 50 μl, each in 3× fold serial dilutions, were added to cells to a final concentration from 670 nM to 30 pM. Recombinant human TPO protein and romiplostim (Amgen Inc.), diluted similarly, were used as positive controls for the assay and the final concentrations were from 0.06 pM to 1.25 nM and 0.2 pM to 4.2 nM, respectively. Cells were cultured at 37° C. with 5% CO₂ for 48 hrs and viability assessed by the CellTiter-Glo kit (Promega). Cell culture plates were read using LMAXII (Molecular devices) and data were analyzed by the GraphPad Prism 5 software (GraphPad Inc). The results indicated that none of the 6 mAbs from Table 4 showed any proliferation/agonistic effect on 32D-hu-c-Mpl cells.

To evaluate the antagonistic activity of the anti-hu-c-mpl mAbs, 9 different concentrations of each mAb, 50 μL each in 3× fold serial dilutions, were added to cells to a final concentration of 0.17 pM to 66.7 nM (1.5 ng/ml to 10 μg/ml), except for 1.169, which was tested from 1 μM to 1.7 pM (15 ng/ml to 100 μg/ml) in 4× fold serial dilution. Cells were incubated with the Abs for 1 hour at 37° C. and rhuTPO was then added to a final concentration of 26 pM (or 2 ng/ml) and cell growth was measured by CellTiter Glow as described above. A neutralizing antibody against TPO (Amgen) was used as positive control.

The results are set forth in Tables 5 & 6 below and indicate that each of the invention mAbs set forth in Table 4 (e.g., mAbs: 1.75; 1.6; 1.111; 1.78; 1.36; and 1.169), neutralized/antagonize/inhibited the proliferation of 32D-hu-c-Mpl cells. The respective IC50 values for each mAb are set forth in Tables 5 & 6. As evident from Tables 5 & 6 the invention anti-hu-c-mpl mAbs have inhibition constants ranging from low picomolar up to low nanomolar sensitivity.

TABLE 5

| | mAb | | |
|---|---|---|---|
| | 1.169 | 1.36 | 1.78 |
| IC50 (nM) | 5.32 | 1.25 | 0.23 |

TABLE 6

| | mAb | | |
|---|---|---|---|
| | 1.111 | 1.6 | 1.75 |
| IC50 (nM) | 0.12 | 0.03 | 0.05 |

Each reference cited herein is incorporated by reference in its entirety for all that it teaches and for all purposes.

The present invention is not to be limited in scope by the specific embodiments described herein, which are intended as single illustrations of individual aspects of the invention, and functionally equivalent methods and components are invention. Indeed, various modifications of the invention, in addition to those shown and described herein will become apparent to those skilled in the art from the foregoing description and accompanying drawings. Such modifications are intended to fall within the scope of the appended claims.

What is claimed is:

1. An isolated antagonistic anti-c-MPL antibody, wherein said antibody competes for binding with at least one antibody selected from the group consisting of mAb-1.75, mAb-1.6, and mAb-1.111.

2. The antibody of claim 1, wherein said antibody competes for binding with at least two antibodies selected from the group consisting of mAb-1.75, mAb-1.6, and mAb-1.111.

3. The antibody of claim 2, wherein said antibody competes for binding with each of mAb-1.75, mAb-1.6, and mAb-1.111.

4. The antibody of claim 1, wherein said antibody binds to the epitope corresponding to -PWQDGPK- (SEQ ID NO: 97).

5. An isolated antagonistic anti-c-MPL antibody, wherein said antibody competes for binding with at least one antibody selected from the group consisting of mAb-1.78, mAb-1.36, and mAb-1.169.

6. The antibody of claim 5, wherein said antibody competes for binding with at least two antibodies selected from the group consisting of mAb-1.78, mAb-1.36, and mAb-1.169.

7. The antibody of claim 6, wherein said antibody competes for binding with each of mAb-1.78, mAb-1.36, and mAb-1.169.

8. The antibody of claim 1, wherein said antibody has a K_{D} of greater than at least 700 pM.

9. The antibody of claim 8, wherein said antibody has a K_{D} of greater than at least 500 pM.

10. The antibody of claim 9, wherein said antibody has a K_{D} of greater than at least 100 pM.

11. The antibody of claim 1, wherein the antibody or the fragment specifically binds to c-MPL and has at least two or more of the characteristics selected from the group consisting of:
(a) affinity (kD) greater than at least 500 pM to c-MPL;
(b) affinity (kD) greater than at least 100 pM to c-MPL;
(c) reduces the growth of human bone marrow (CD34+ DC38−) progenitor cells in a mammal by at least 50%; and
(d) binds the extracellular domain of c-MPL.

12. The antibody of claim 1, or fragment of an antibody, wherein the antibody or the fragment specifically binds to human c-MPL and comprises (A-1/mAb1.6):
(a) a VH CDR1 having an amino acid sequence identical to or comprising 1, 2, or 3 amino acid residue substitutions relative to SEQ ID NO: 20;
(b) a VH CDR2 having an amino acid sequence identical to or comprising 1, 2, or 3 amino acid residue substitutions relative to SEQ ID NO: 22;
(c) a VH CDR3 having an amino acid sequence identical to or comprising 1, 2, or 3 amino acid residue substitutions relative to SEQ ID NO: 24;
(d) a VL CDR1 having an amino acid sequence identical to or comprising 1, 2, or 3 amino acid residue substitutions relative to SEQ ID NO: 2;
(e) a VL CDR2 having an amino acid sequence identical to or comprising 1, 2, or 3 amino acid residue substitutions relative to SEQ ID NO: 4; and
(f) a VL CDR3 having an amino acid sequence identical to or comprising 1, 2, or 3 amino acid residue substitutions relative to SEQ ID NO: 6.

13. The antibody or fragment of an antibody of claim 12, wherein the antibody or the fragment specifically binds to human c-MPL and comprises:
(a) a VH CDR1 having the amino acid sequence of SEQ ID NO: 20;
(b) a VH CDR2 having the amino acid sequence of SEQ ID NO: 22
(c) a VH CDR3 having the amino acid sequence of SEQ ID NO: 24;
(d) a VL CDR1 having the amino acid sequence of SEQ ID NO: 2;
(e) a VL CDR2 having the amino acid sequence of SEQ ID NO: 4; and
(f) a VL CDR3 having the amino acid sequence of SEQ ID NO: 6.

14. The antibody of claim 1, or antibody fragment, wherein the antibody or the fragment (i) comprises a VH chain domain comprising three CDRs and a VL chain domain comprising three CDRs; and (ii) specifically binds human c-MPL, wherein the three CDRs of the VH chain domain comprise:
(a) a VH CDR1 comprising the amino acid sequence of SEQ ID NO: 20;
(b) a VH CDR2 comprising the amino acid sequence of SEQ ID NO: 22; and
(c) a VH CDR3 comprising the amino acid sequence of SEQ ID NO: 24.

15. The antibody of claim 1, or antibody fragment, wherein the antibody or the fragment (i) comprises a VH chain domain comprising three CDRs and a VL chain domain comprising three CDRs; and (ii) specifically binds human c-MPL, wherein the three CDRs of the VL chain domain comprise:
(a) a VL CDR1 comprising the amino acid sequence of SEQ ID NO: 2;
(b) a VL CDR2 comprising the amino acid sequence of SEQ ID NO: 4; and
(c) a VL CDR3 comprising the amino acid sequence of SEQ ID NO: 6.

16. The antibody of claim 1, or antibody fragment, wherein the antibody or the fragment specifically binds human c-MPL and comprises a heavy chain variable domain having at least 90% identity to the amino acid of SEQ ID NO: 40 and comprises a light chain variable domain having at least 90% identity to the amino acid sequence of SEQ ID NO: 38, wherein said antibody has the activity of antagonizing human-c-MPL.

17. The antibody or antibody fragment of claim 16, wherein the antibody or the fragment comprises the heavy chain variable domain of SEQ ID NO: 40 and the light chain variable domain of SEQ ID NO: 38.

18. The antibody of claim 1, or fragment of an antibody, wherein the antibody or the fragment specifically binds to human c-MPL and comprises (A-2/mAb 1.75):
(a) a VH CDR1 having an amino acid sequence identical to or comprising 1, 2, or 3 amino acid residue substitutions relative to SEQ ID NO: 26;
(b) a VH CDR2 having an amino acid sequence identical to or comprising 1, 2, or 3 amino acid residue substitutions relative to SEQ ID NO: 28;
(c) a VH CDR3 having an amino acid sequence identical to or comprising 1, 2, or 3 amino acid residue substitutions relative to SEQ ID NO: 30;
(d) a VL CDR1 having an amino acid sequence identical to or comprising 1, 2, or 3 amino acid residue substitutions relative to SEQ ID NO: 8;
(e) a VL CDR2 having an amino acid sequence identical to or comprising 1, 2, or 3 amino acid residue substitutions relative to SEQ ID NO: 10; and
(f) a VL CDR3 having an amino acid sequence identical to or comprising 1, 2, or 3 amino acid residue substitutions relative to SEQ ID NO: 12.

19. The antibody or fragment of an antibody of claim 18, wherein the antibody or the fragment specifically binds to human c-MPL and comprises:
(a) a VH CDR1 having the amino acid sequence of SEQ ID NO: 26;
(b) a VH CDR2 having the amino acid sequence of SEQ ID NO: 28;
(c) a VH CDR3 having the amino acid sequence of SEQ ID NO: 30;
(d) a VL CDR1 having the amino acid sequence of SEQ ID NO: 8;
(e) a VL CDR2 having the amino acid sequence of SEQ ID NO: 10; and
(f) a VL CDR3 having the amino acid sequence of SEQ ID NO: 12.

20. The antibody of claim 1, or antibody fragment, wherein the antibody or the fragment (i) comprises a VH chain domain comprising three CDRs and a VL chain domain comprising three CDRs; and (ii) specifically binds human c-MPL, wherein the three CDRs of the VH chain domain comprise:
(a) a VH CDR1 comprising the amino acid sequence of SEQ ID NO: 26;
(b) a VH CDR2 comprising the amino acid sequence of SEQ ID NO: 28; and
(c) a VH CDR3 comprising the amino acid sequence of SEQ ID NO: 30.

21. The antibody of claim 1, or antibody fragment, wherein the antibody or the fragment (i) comprises a VH chain domain comprising three CDRs and a VL chain domain comprising three CDRs; and (ii) specifically binds human c-MPL, wherein the three CDRs of the VL chain domain comprise:
(a) a VL CDR1 comprising the amino acid sequence of SEQ ID NO: 8;
(b) a VL CDR2 comprising the amino acid sequence of SEQ ID NO: 10; and
(c) a VL CDR3 comprising the amino acid sequence of SEQ ID NO: 12.

22. The antibody of claim 1, or antibody fragment, wherein the antibody or the fragment specifically binds human c-MPL and comprises a heavy chain variable domain having at least 90% identity to the amino acid of SEQ ID NO: 44 and comprises a light chain variable domain having at least 90% identity to the amino acid sequence of SEQ ID NO:42, wherein said antibody has the activity of antagonizing human-c-MPL.

23. The antibody or antibody fragment of claim 22, wherein the antibody or the fragment comprises the heavy chain variable domain of SEQ ID NO: 44 and the light chain variable domain of SEQ ID NO:42.

24. The antibody of claim 5, or fragment of an antibody, wherein the antibody or the fragment specifically binds to human c-MPL and comprises (A-3/mAb 1.78):
(a) a VH CDR1 having an amino acid sequence identical to or comprising 1, 2, or 3 amino acid residue substitutions relative to SEQ ID NO: 32;
(b) a VH CDR2 having an amino acid sequence identical to or comprising 1, 2, or 3 amino acid residue substitutions relative to SEQ ID NO: 34;
(c) a VH CDR3 having an amino acid sequence identical to or comprising 1, 2, or 3 amino acid residue substitutions relative to SEQ ID NO: 36;
(d) a VL CDR1 having an amino acid sequence identical to or comprising 1, 2, or 3 amino acid residue substitutions relative to SEQ ID NO: 14;
(e) a VL CDR2 having an amino acid sequence identical to or comprising 1, 2, or 3 amino acid residue substitutions relative to SEQ ID NO: 16; and
(f) a VL CDR3 having an amino acid sequence identical to or comprising 1, 2, or 3 amino acid residue substitutions relative to SEQ ID NO: 18.

25. The antibody or fragment of an antibody of claim 24, wherein the antibody or the fragment specifically binds to human c-MPL and comprises:
(a) a VH CDR1 having the amino acid sequence of SEQ ID NO:32;
(b) a VH CDR2 having the amino acid sequence of SEQ ID NO: 34;
(c) a VH CDR3 having the amino acid sequence of SEQ ID NO: 36;
(d) a VL CDR1 having the amino acid sequence of SEQ ID NO: 14;
(e) a VL CDR2 having the amino acid sequence of SEQ ID NO: 16; and
(f) a VL CDR3 having the amino acid sequence of SEQ ID NO: 18.

26. The antibody of claim 5, or antibody fragment, wherein the antibody or the fragment (i) comprises a VH chain domain comprising three CDRs and a VL chain domain comprising three CDRs; and (ii) specifically binds human c-MPL, wherein the three CDRs of the VH chain domain comprise:
(a) a VH CDR1 comprising the amino acid sequence of SEQ ID NO: 32;
(b) a VH CDR2 comprising the amino acid sequence of SEQ ID NO: 34; and
(c) a VH CDR3 comprising the amino acid sequence of SEQ ID NO: 36.

27. The antibody of claim 5, or antibody fragment, wherein the antibody or the fragment (i) comprises a VH chain domain comprising three CDRs and a VL chain domain comprising three CDRs; and (ii) specifically binds human c-MPL, wherein the three CDRs of the VL chain domain comprise:
(a) a VL CDR1 comprising the amino acid sequence of SEQ ID NO: 14;
(b) a VL CDR2 comprising the amino acid sequence of SEQ ID NO: 16 and
(c) a VL CDR3 comprising the amino acid sequence of SEQ ID NO: 18.

28. The antibody of claim 5, or antibody fragment, wherein the antibody or the fragment specifically binds human c-MPL and comprises a heavy chain variable domain having at least 90% identity to the amino acid of SEQ ID NO: 48 and comprises a light chain variable domain having at least 90% identity to the amino acid sequence of SEQ ID NO: 46, wherein said antibody has the activity of antagonizing human-c-MPL.

29. The antibody or antibody fragment of claim 28, wherein the antibody or the fragment comprises the heavy chain variable domain of SEQ ID NO: 48 and the light chain variable domain of SEQ ID NO: 46.

30. The antibody of claim 1, or fragment of an antibody, wherein the antibody or the fragment specifically binds to human c-MPL and comprises (A-4/mAb1.111):
(a) a VH CDR1 having an amino acid sequence identical to or comprising 1, 2, or 3 amino acid residue substitutions relative to SEQ ID NO: 68;
(b) a VH CDR2 having an amino acid sequence identical to or comprising 1, 2, or 3 amino acid residue substitutions relative to SEQ ID NO: 70;
(c) a VH CDR3 having an amino acid sequence identical to or comprising 1, 2, or 3 amino acid residue substitutions relative to SEQ ID NO: 72;
(d) a VL CDR1 having an amino acid sequence identical to or comprising 1, 2, or 3 amino acid residue substitutions relative to SEQ ID NO: 50;
(e) a VL CDR2 having an amino acid sequence identical to or comprising 1, 2, or 3 amino acid residue substitutions relative to SEQ ID NO: 52; and
(f) a VL CDR3 having an amino acid sequence identical to or comprising 1, 2, or 3 amino acid residue substitutions relative to SEQ ID NO: 54.

31. The antibody or fragment of an antibody of claim 30, wherein the antibody or the fragment specifically binds to human c-MPL and comprises:
(a) a VH CDR1 having the amino acid sequence of SEQ ID NO: 68;
(b) a VH CDR2 having the amino acid sequence of SEQ ID NO: 70
(c) a VH CDR3 having the amino acid sequence of SEQ ID NO: 72;
(d) a VL CDR1 having the amino acid sequence of SEQ ID NO: 50;
(e) a VL CDR2 having the amino acid sequence of SEQ ID NO: 52; and
(f) a VL CDR3 having the amino acid sequence of SEQ ID NO: 54.

32. The antibody of claim 1, or antibody fragment, wherein the antibody or the fragment (i) comprises a VH chain domain comprising three CDRs and a VL chain domain comprising three CDRs; and (ii) specifically binds human c-MPL, wherein the three CDRs of the VH chain domain comprise:
(a) a VH CDR1 comprising the amino acid sequence of SEQ ID NO: 68;
(b) a VH CDR2 comprising the amino acid sequence of SEQ ID NO: 70; and
(c) a VH CDR3 comprising the amino acid sequence of SEQ ID NO: 72.

33. The antibody of claim 1, or antibody fragment, wherein the antibody or the fragment (i) comprises a VH chain domain comprising three CDRs and a VL chain domain comprising three CDRs; and (ii) specifically binds human c-MPL, wherein the three CDRs of the VL chain domain comprise:
(a) a VL CDR1 comprising the amino acid sequence of SEQ ID NO: 50;
(b) a VL CDR2 comprising the amino acid sequence of SEQ ID NO: 52; and
(c) a VL CDR3 comprising the amino acid sequence of SEQ ID NO: 54.

34. The antibody of claim 1, or antibody fragment, wherein the antibody or the fragment specifically binds human c-MPL and comprises a heavy chain variable domain having at least 90% identity to the amino acid of SEQ ID NO: 88 and comprises a light chain variable domain having at least 90% identity to the amino acid sequence of SEQ ID NO: 86, wherein said antibody has the activity of antagonizing human-c-MPL.

35. The antibody or antibody fragment of claim 34, wherein the antibody or the fragment comprises the heavy chain variable domain of SEQ ID NO: 88 and the light chain variable domain of SEQ ID NO: 86.

36. The antibody of claim 5, or fragment of an antibody, wherein the antibody or the fragment specifically binds to human c-MPL and comprises (A-5/mAb 1.36):
(a) a VH CDR1 having an amino acid sequence identical to or comprising 1, 2, or 3 amino acid residue substitutions relative to SEQ ID NO: 74;
(b) a VH CDR2 having an amino acid sequence identical to or comprising 1, 2, or 3 amino acid residue substitutions relative to SEQ ID NO: 76;
(c) a VH CDR3 having an amino acid sequence identical to or comprising 1, 2, or 3 amino acid residue substitutions relative to SEQ ID NO: 78;
(d) a VL CDR1 having an amino acid sequence identical to or comprising 1, 2, or 3 amino acid residue substitutions relative to SEQ ID NO: 56;
(e) a VL CDR2 having an amino acid sequence identical to or comprising 1, 2, or 3 amino acid residue substitutions relative to SEQ ID NO: 58; and
(f) a VL CDR3 having an amino acid sequence identical to or comprising 1, 2, or 3 amino acid residue substitutions relative to SEQ ID NO: 60.

37. The antibody or fragment of an antibody of claim 36, wherein the antibody or the fragment specifically binds to human c-MPL and comprises:
(a) a VH CDR1 having the amino acid sequence of SEQ ID NO: 74;
(b) a VH CDR2 having the amino acid sequence of SEQ ID NO: 76;
(c) a VH CDR3 having the amino acid sequence of SEQ ID NO: 78;
(d) a VL CDR1 having the amino acid sequence of SEQ ID NO: 56;
(e) a VL CDR2 having the amino acid sequence of SEQ ID NO: 58; and
(f) a VL CDR3 having the amino acid sequence of SEQ ID NO: 60.

38. The antibody of claim 5, or antibody fragment, wherein the antibody or the fragment (i) comprises a VH chain domain comprising three CDRs and a VL chain domain comprising three CDRs; and (ii) specifically binds human c-MPL, wherein the three CDRs of the VH chain domain comprise:
(a) a VH CDR1 comprising the amino acid sequence of SEQ ID NO: 74;
(b) a VH CDR2 comprising the amino acid sequence of SEQ ID NO: 76; and
(c) a VH CDR3 comprising the amino acid sequence of SEQ ID NO: 78.

39. The antibody of claim 5, or antibody fragment, wherein the antibody or the fragment (i) comprises a VH chain domain comprising three CDRs and a VL chain domain comprising three CDRs; and (ii) specifically binds human c-MPL, wherein the three CDRs of the VL chain domain comprise:
(a) a VL CDR1 comprising the amino acid sequence of SEQ ID NO: 56;
(b) a VL CDR2 comprising the amino acid sequence of SEQ ID NO: 58; and
(c) a VL CDR3 comprising the amino acid sequence of SEQ ID NO: 60.

40. The antibody of claim 5, or antibody fragment, wherein the antibody or the fragment specifically binds human c-MPL and comprises a heavy chain variable domain having at least 90% identity to the amino acid of SEQ ID NO: 92 and comprises a light chain variable domain having at least 90% identity to the amino acid sequence of SEQ ID NO:90, wherein said antibody has the activity of antagonizing human-c-MPL.

41. The antibody or antibody fragment of claim 40, wherein the antibody or the fragment comprises the heavy chain variable domain of SEQ ID NO: 92 and the light chain variable domain of SEQ ID NO:90.

42. The antibody of claim 5, or fragment of an antibody, wherein the antibody or the fragment specifically binds to human c-MPL and comprises (A-6/mAb 1.169):
(a) a VH CDR1 having an amino acid sequence identical to or comprising 1, 2, or 3 amino acid residue substitutions relative to SEQ ID NO: 80;
(b) a VH CDR2 having an amino acid sequence identical to or comprising 1, 2, or 3 amino acid residue substitutions relative to SEQ ID NO: 82;
(c) a VH CDR3 having an amino acid sequence identical to or comprising 1, 2, or 3 amino acid residue substitutions relative to SEQ ID NO: 84;
(d) a VL CDR1 having an amino acid sequence identical to or comprising 1, 2, or 3 amino acid residue substitutions relative to SEQ ID NO: 62;
(e) a VL CDR2 having an amino acid sequence identical to or comprising 1, 2, or 3 amino acid residue substitutions relative to SEQ ID NO: 64; and
(f) a VL CDR3 having an amino acid sequence identical to or comprising 1, 2, or 3 amino acid residue substitutions relative to SEQ ID NO: 66.

43. The antibody or fragment of an antibody of claim 42, wherein the antibody or the fragment specifically binds to human c-MPL and comprises:
(a) a VH CDR1 having the amino acid sequence of SEQ ID NO:80;
(b) a VH CDR2 having the amino acid sequence of SEQ ID NO: 82;
(c) a VH CDR3 having the amino acid sequence of SEQ ID NO: 84;
(d) a VL CDR1 having the amino acid sequence of SEQ ID NO: 62;
(e) a VL CDR2 having the amino acid sequence of SEQ ID NO: 64; and
(f) a VL CDR3 having the amino acid sequence of SEQ ID NO: 66.

44. The antibody of claim 5, or antibody fragment, wherein the antibody or the fragment (i) comprises a VH chain domain comprising three CDRs and a VL chain domain comprising three CDRs; and (ii) specifically binds human c-MPL, wherein the three CDRs of the VH chain domain comprise:
(a) a VH CDR1 comprising the amino acid sequence of SEQ ID NO: 80;
(b) a VH CDR2 comprising the amino acid sequence of SEQ ID NO: 82; and
(c) a VH CDR3 comprising the amino acid sequence of SEQ ID NO: 84.

45. The antibody of claim 5, or antibody fragment, wherein the antibody or the fragment (i) comprises a VH chain domain comprising three CDRs and a VL chain domain comprising three CDRs; and (ii) specifically binds human c-MPL, wherein the three CDRs of the VL chain domain comprise:
(a) a VL CDR1 comprising the amino acid sequence of SEQ ID NO: 62;
(b) a VL CDR2 comprising the amino acid sequence of SEQ ID NO: 64 and
(c) a VL CDR3 comprising the amino acid sequence of SEQ ID NO: 66.

46. The antibody of claim 5, or antibody fragment, wherein the antibody or the fragment specifically binds human c-MPL and comprises a heavy chain variable domain having at least 90% identity to the amino acid of SEQ ID NO: 96 and comprises a light chain variable domain having at least 90% identity to the amino acid sequence of SEQ ID NO: 94, wherein said antibody has the activity of antagonizing human-c-MPL.

47. The antibody or antibody fragment of claim 46, wherein the antibody or the fragment comprises the heavy chain variable domain of SEQ ID NO: 96 and the light chain variable domain of SEQ ID NO: 94.

48. The antibody of claim 1 wherein said antibody further binds cynomolgus MPL.

49. The antibody of claim 1 wherein said antibody does not compete for binding with rhuTPO.

50. The antibody of claim 1, wherein said antibody is selected from the group consisting of a human antibody, a humanized antibody, chimeric antibody, a monoclonal antibody, a polyclonal antibody, a recombinant antibody, an antigen-binding antibody fragment, a single chain antibody, a diabody, a triabody, a tetrabody, a Fab fragment, an F(fa′)x fragment, a domain antibody, an IgD antibody, an IgE antibody, and IgM antibody, and IgG1 antibody, and IgG2 antibody, and IgG3 antibody, and IgG4 antibody, and IgG4 antibody having at least one mutation in the hinge region.

51. The antibody of claim 50, wherein the antibody is a monoclonal antibody.

52. The antibody of claim 50, wherein the antibody is a chimeric antibody, a humanized antibody, or a fully human antibody.

53. A sterile composition comprising the antibody of claim 1.

54. The composition of claim 53 further comprising a pharmaceutically acceptable carrier.

55. An isolated nucleic acid encoding the antibody of claim 1.

56. An expression vector comprising the nucleic acid of claim 55.

57. An isolated cell comprising the vector of claim 56.

58. The isolated cell of claim 57, wherein the chromosome of the cell comprises the nucleic acid.

59. The isolated cell of claim 57, wherein the cell is a hybridoma.

60. A method of producing an antigen binding protein that specifically binds to the human c-MPL protein comprising incubating the cell of claim 57 under conditions that allow it to express the antigen binding protein.

61. A method of inhibiting tumor cell proliferation in an animal, comprising administering to said animal a therapeutically effective dose of the antibody of claim 1.

62. A method of preventing, treating, or managing cancer in an animal in need thereof, said method comprising administering to said animal a dose of an effective amount of the composition of claim 53.

63. The method of claim 62, wherein said animal is human.

64. A method for treating, preventing or alleviating the symptoms of a c-MPL mediated disorder in a subject in need thereof comprising administering an effective amount of the antibody of claim 1.

65. A method for inhibiting c-MPL activity in a cell expressing c-MPL, comprising contacting the cell with the antibody of claim 1.

66. A method of determining the presence or absence of human c-MPL (TPO-R), comprising:
(a) contacting a sample with a human c-MPL-specific monoclonal antibody, or a functional fragment thereof, under conditions sufficient for binding, and
(b) measuring binding of said human c-MPL-specific monoclonal antibody, or functional fragment thereof, wherein binding of said human c-MPL-specific monoclonal antibody, or functional fragment thereof, indicates the presence of human c-MPL, and wherein an absence of binding of said human c-MPL-specific monoclonal antibody, or functional fragment thereof, indicates the absence of human c-MPL.

67. The method of claim 66, wherein said sample comprises a cell, cell lysate, tissue or organ.

68. The method of claim 66, wherein said human c-MPL-specific monoclonal antibody, or functional fragment thereof, comprises generally a Kd value less than or equal to a Kd selected from the group consisting of: 100 nM; 90; nM; 80 nM; 70 nM; 60 nM; 50 nM; 40 nM; 30 nM; 20 nM; 10 nM; 5 nM; 4 nM; 3 nM; 2 nM; 1 nM (e.g., 1000 pM); 900, pM; 800 pM; 700 pM; 600 pM; 500 pM; 400 pM; 300 pM; 200 pM; 100 pM; 50 pM; 25 pM; 20 pM; 15 pM; 10 pM; 5 pM; 3 pM or 1 pM.

69. The method of claim 66, wherein said human c-MPL-specific monoclonal antibody, or functional fragment thereof, inhibits binding to human c-MPL of a monoclonal antibody selected from the group consisting of 1.6; 1.75; 1.78; 1.111; 1.36; and 1.169.

70. The method of claim 66, wherein said human c-MPL-specific antibody, or functional fragment thereof, is selected from the monoclonal antibodies consisting of 1.6; 1.75; 1.78; 1.111; 1.36; and 1.169.

71. The method of claim 66, wherein said functional fragment comprises an antibody binding fragment selected from Fd, Fv, Fab, F(ab′), F(ab)₂, F(ab′)₂ and scFv.

72. A method of determining susceptibility of a cell to c-MPL-mediated proliferation, comprising:
(a) contacting a cellular sample with a human c-MPL-specific monoclonal antibody, or functional fragment thereof, under conditions sufficient for binding, and
(b) measuring the binding of said human c-MPL-specific monoclonal antibody, or functional fragment thereof, wherein the presence of human c-MPL indicates susceptibility of said cellular sample to human c-MPL-mediated proliferation.

73. The method of claim 72 wherein said cellular sample comprises a cell, tissue or organ.

74. The method of claim 72, wherein said human c-MPL-specific monoclonal antibody, or functional fragment thereof, comprises generally a Kd value less than or equal to a Kd selected from the group consisting of: 100 nM; 90; nM; 80 nM; 70 nM; 60 nM; 50 nM; 40 nM; 30 nM; 20 nM; 10 nM; 5 nM; 4 nM; 3 nM; 2 nM; 1 nM (e.g., 1000 pM); 500 pM; 400 pM; 300 pM; 200 pM; 100 pM; 50 pM; 25 pM; 20 pM; 15 pM; 10 pM; 5 pM; 3 pM or 1 pM.

75. The method of claim 72, wherein said human c-MPL-specific monoclonal antibody, or functional fragment thereof, inhibits binding to human c-MPL of a monoclonal antibody selected from the group consisting of 1.6; 1.75; 1.78; 1.111; 1.36; and 1.169.

76. The method of claim 72 wherein said human c-MPL-specific antibody, or functional fragment thereof, is selected from the monoclonal antibodies consisting of 1.6; 1.75; 1.78; 1.111; 1.36; and 1.169.

77. The method of claim 72, wherein said functional fragment comprises an antibody binding fragment selected from Fd, Fv, Fab, F(ab′), F(ab)₂, F(ab′)₂ and scFv.

78. A kit for determining the presence or absence of human c-MPL, comprising:
(a) a monoclonal antibody having specific binding activity to human c-MPL, or a functional fragment thereof, and
(b) a detection reagent.

79. The kit of claim 77, wherein said antibody, or functional fragment thereof, inhibits the binding to human c-MPL of a monoclonal antibody selected from the group consisting of 1.6; 1.75; 1.78; 1.111; 1.36; and 1.169.

80. The kit of claim 78, wherein said antibody, or functional fragment thereof, is selected from the monoclonal antibodies consisting of 1.6; 1.75; 1.78; 1.111; 1.36; and 1.169.

81. The kit of claim 78, wherein said functional fragment comprises an antibody binding fragment selected from Fd, Fv, Fab, F(ab′), F(ab)₂, F(ab′)₂ and scFv.

82. The kit of claim 78, further comprising a polypeptide having a human c-MPL extracellular domain, a cell line expressing human c-MPL.

83. The kit of claim 78, further comprising an ancillary reagent.

84. The kit of claim 78, further comprising a c-MPL agonist.

85. The kit of claim 84, wherein said c-MPL agonist comprises TPO.

86. An antibody that binds to the c-MPL epitope corresponding to -PWQDGPK- (SEQ ID NO: 97).


SEQUENCE LISTING (99 sequences)

SEQ ID NO: 1 - DNA, 33 bp, Mus musculus
  CDS (1)..(33)
atggcaagtgaggacatttatattcgcttagcc

SEQ ID NO: 2 - protein, 11 aa, Mus musculus
MASEDIYIRLA

SEQ ID NO: 3 - DNA, 21 bp, Mus musculus
  CDS (1)..(21)
gctgcaaccagtttggaaact

SEQ ID NO: 4 - protein, 7 aa, Mus musculus
AATSLET

SEQ ID NO: 5 - DNA, 27 bp, Mus musculus
  CDS (1)..(27)
caacagtattggagttctccgtggacg

SEQ ID NO: 6 - protein, 9 aa, Mus musculus
QQYWSSPWT

SEQ ID NO: 7 - DNA, 30 bp, Mus musculus
  CDS (1)..(30)
agtgccagctcaagtgtaagttacatgtac

SEQ ID NO: 8 - protein, 10 aa, Mus musculus
SASSSVSYMY

SEQ ID NO: 9 - DNA, 21 bp, Mus musculus
  CDS (1)..(21)
cgcacatccaacctggcttct

SEQ ID NO: 10 - protein, 7 aa, Mus musculus
RTSNLAS

SEQ ID NO: 11 - DNA, 27 bp, Mus musculus
  CDS (1)..(27)
cagcagtatcatagttacccaaccacg

SEQ ID NO: 12 - protein, 9 aa, Mus musculus
QQYHSYPTT

SEQ ID NO: 13 - DNA, 48 bp, Mus musculus
  CDS (1)..(48)
aggtctgataagagtctcctgcatagtaatggcaacacttacttgttt

SEQ ID NO: 14 - protein, 16 aa, Mus musculus
RSDKSLLHSNGNTYLF

SEQ ID NO: 15 - DNA, 21 bp, Mus musculus
  CDS (1)..(21)
cggatgtccaaccttgcctca

SEQ ID NO: 16 - protein, 7 aa, Mus musculus
RMSNLAS

SEQ ID NO: 17 - DNA, 27 bp, Mus musculus
  CDS (1)..(27)
atgcaacatctagaatatccgtacacg

SEQ ID NO: 18 - protein, 9 aa, Mus musculus
MQHLEYPYT

SEQ ID NO: 19 - DNA, 15 bp, Mus musculus
  CDS (1)..(15)
gattatacctggaac

SEQ ID NO: 20 - protein, 5 aa, Mus musculus
DYTWN

SEQ ID NO: 21 - DNA, 48 bp, Mus musculus
  CDS (1)..(48)
tacataacttacagtggtagcactagctacaacccatctctcaaaagt

SEQ ID NO: 22 - protein, 16 aa, Mus musculus
YITYSGSTSYNPSLKS

SEQ ID NO: 23 - DNA, 27 bp, Mus musculus
  CDS (1)..(27)
ctgggacgtcgctatactatggactac

SEQ ID NO: 24 - protein, 9 aa, Mus musculus
LGRRYTMDY

SEQ ID NO: 25 - DNA, 15 bp, Mus musculus
  CDS (1)..(15)
acctacgccatgaac

SEQ ID NO: 26 - protein, 5 aa, Mus musculus
TYAMN

SEQ ID NO: 27 - DNA, 57 bp, Mus musculus
  CDS (1)..(57)
cacataagaagtaaaagtaataattttgcaacatattatgccgattcagtgaaagac

SEQ ID NO: 28 - protein, 19 aa, Mus musculus
HIRSKSNNFATYYADSVKD

SEQ ID NO: 29 - DNA, 27 bp, Mus musculus
  CDS (1)..(27)
caagggggtgactttcctatggactac

SEQ ID NO: 30 - protein, 9 aa, Mus musculus
QGGDFPMDY

SEQ ID NO: 31 - DNA, 15 bp, Mus musculus
  CDS (1)..(15)
agttcctggttgaac

SEQ ID NO: 32 - protein, 5 aa, Mus musculus
SSWLN

SEQ ID NO: 33 - DNA, 51 bp, Mus musculus
  CDS (1)..(51)
cggatttatcctggagatggagaaaatcactataatgggaaattcaagggc

SEQ ID NO: 34 - protein, 17 aa, Mus musculus
RIYPGDGENHYNGKFKG

SEQ ID NO: 35 - DNA, 18 bp, Mus musculus
  CDS (1)..(18)
tattatgaagggggttat

SEQ ID NO: 36 - protein, 6 aa, Mus musculus
YYEGGY

SEQ ID NO: 37 - DNA, 321 bp, Mus musculus
  CDS (1)..(321)
gacatccagatgacacaatcttcatcctccttttctgtatctctaggagacagagtcaccattacttgcatggcaagtgaggacatttatattcgcttagcctggtatcagcagaaaccaggaaatgctcctaggctcttaatatctgctgcaaccagtttggaaactggggttccttctagattcagtggcagtggatctggaaaggattacactctcagcattaccagtctacagactgaagatgttggtacatattactgtcaacagtattggagttctccgtggacgttcggtggaggcaccaagctggaaatcaat

SEQ ID NO: 38 - protein, 107 aa, Mus musculus
DIQMTQSSSSFSVSLGDRVTITCMASEDIYIRLAWYQQKPGNAPRLLISAATSLETGVPSRFSGSGSGKDYTLSITSLQTEDVGTYYCQQYWSSPWTFGGGTKLEIN

SEQ ID NO: 39 - DNA, 354 bp, Mus musculus
  CDS (1)..(354)
gatgtgcagcttcaggagtcgggacctggcctggtgaaaccttctcagtctctgtccctcacctgcactgtcactggctactcaatcaccagtgattatacctggaactggatccggcagtttccaggaaacaaactggagtggatgggctacataacttacagtggtagcactagctacaacccatctctcaaaagtcgaaactctatcactcgagacacatccaagaaccagttcttcctgcagttgaattctgtgactactgaggacacagccacatattactgtgcaagactgggacgtcgctatactatggactactggggtcaaggaacctcagtcaccgtctcctca

SEQ ID NO: 40 - protein, 118 aa, Mus musculus
DVQLQESGPGLVKPSQSLSLTCTVTGYSITSDYTWNWIRQFPGNKLEWMGYITYSGSTSYNPSLKSRNSITRDTSKNQFFLQLNSVTTEDTATYYCARLGRRYTMDYWGQGTSVTVSS

SEQ ID NO: 41 - DNA, 318 bp, Mus musculus
  CDS (1)..(318)
caaattgttctcacccagtctccagcaatcatgtctgcatctccaggggagaaggtcaccatatcctgcagtgccagctcaagtgtaagttacatgtactggtaccagcagaagccaggatcctcccccaaaccctggatttatcgcacatccaacctggcttctggagtccctgctcgcttcagtggcagtgggtctgggacctcttactctctcacaatcagcaacatggaggctgaagatgctgccgcttattactgccagcagtatcatagttacccaaccacgttcggaggggggaccaagctggaagtgaaa

SEQ ID NO: 42 - protein, 106 aa, Mus musculus
QIVLTQSPAIMSASPGEKVTISCSASSSVSYMYWYQQKPGSSPKPWIYRTSNLASGVPARFSGSGSGTSYSLTISNMEAEDAAAYYCQQYHSYPTTFGGGTKLEVK

SEQ ID NO: 43 - DNA, 360 bp, Mus musculus
  CDS (1)..(360)
gaggtgcagcttgttgagtctggtggaggattggtgcagcctaaagggtcattgaaactctcatgtgcagcctctggattcagcttcaatacctacgccatgaactgggtccgccaggctccaggaaagggtttggaatggattgctcacataagaagtaaaagtaataattttgcaacatattatgccgattcagtgaaagacagattcagcatctccagagatgcttcagaaaacattctctttctgcaaatgaacaacttgaaaactgaggacacagccatgtattattgtgtgagacaagggggtgactttcctatggactactggggtcaaggaacctcagtcaccgtctcctca

SEQ ID NO: 44 - protein, 120 aa, Mus musculus
EVQLVESGGGLVQPKGSLKLSCAASGFSFNTYAMNWVRQAPGKGLEWIAHIRSKSNNFATYYADSVKDRFSISRDASENILFLQMNNLKTEDTAMYYCVRQGGDFPMDYWGQGTSVTVSS

SEQ ID NO: 45 - DNA, 336 bp, Mus musculus
  CDS (1)..(336)
gatattgtgatgactcaggctgcaccctctattcctgtcactcctggagagtcagtatccatctcctgcaggtctgataagagtctcctgcatagtaatggcaacacttacttgttttggttcctgcagaggccaggccagtctcctcagctcctgatatatcggatgtccaaccttgcctcaggagtcccagacaggttcagtggcagtgggtcaggaactgctttcacactgagaatcagtggagtggaggctgaggatgtgggtgtttattactgtatgcaacatctagaatatccgtacacgttcggaggggggaccaagctggaaataaaa

SEQ ID NO: 46 - protein, 112 aa, Mus musculus
DIVMTQAAPSIPVTPGESVSISCRSDKSLLHSNGNTYLFWFLQRPGQSPQLLIYRMSNLASGVPDRFSGSGSGTAFTLRISGVEAEDVGVYYCMQHLEYPYTFGGGTKLEIK

SEQ ID NO: 47 - DNA, 345 bp, Mus musculus
  CDS (1)..(345)
caggttcaactgcagcagtctggacctgaactggtgaagcctggggcctcagtgaagatgtcctgcaaggcttctggctacgcattcagtagttcctggttgaactgggtgaggcagaggcctggaaagggtcttgagtggattggacggatttatcctggagatggagaaaatcactataatgggaaattcaagggcaaggccacactgactgcagacaaatcctccagcacaggctacatgcaactcagcagcctgacgtctgaggactctgcggtctacttctgtgcaagttattatgaagggggttattggggccaagggactctaatcactgtctctgca

SEQ ID NO: 48 - protein, 115 aa, Mus musculus
QVQLQQSGPELVKPGASVKMSCKASGYAFSSSWLNWVRQRPGKGLEWIGRIYPGDGENHYNGKFKGKATLTADKSSSTGYMQLSSLTSEDSAVYFCASYYEGGYWGQGTLITVSA

SEQ ID NO: 49 - DNA, 33 bp, Mus musculus
  CDS (1)..(33)
aaggcaagtgaggacatatatattcgcttagcc

SEQ ID NO: 50 - protein, 11 aa, Mus musculus
KASEDIYIRLA

SEQ ID NO: 51 - DNA, 21 bp, Mus musculus
  CDS (1)..(21)
gctgcaaccagtttggaaact

SEQ ID NO: 52 - protein, 7 aa, Mus musculus
AATSLET

SEQ ID NO: 53 - DNA, 27 bp, Mus musculus
  CDS (1)..(27)
caacaatattggactactccgtggacg

SEQ ID NO: 54 - protein, 9 aa, Mus musculus
QQYWTTPWT

SEQ ID NO: 55 - DNA, 48 bp, Mus musculus
  CDS (1)..(48)
aggtctagtaagagtctcctgcatagtaatggcaacacttacttgtat

SEQ ID NO: 56 - protein, 16 aa, Mus musculus
RSSKSLLHSNGNTYLY

SEQ ID NO: 57 - DNA, 21 bp, Mus musculus
  CDS (1)..(21)
cggatgtccaaccttgcctca

SEQ ID NO: 58 - protein, 7 aa, Mus musculus
RMSNLAS

SEQ ID NO: 59 - DNA, 27 bp, Mus musculus
  CDS (1)..(27)
atgcaacatctagaatatccgtacacg

SEQ ID NO: 60 - protein, 9 aa, Mus musculus
MQHLEYPYT

SEQ ID NO: 61 - DNA, 48 bp, Mus musculus
  CDS (1)..(48)
aggtctagtaagagtctcctgcatagtaatggcaacacttacttgttt

SEQ ID NO: 62 - protein, 16 aa, Mus musculus
RSSKSLLHSNGNTYLF

SEQ ID NO: 63 - DNA, 21 bp, Mus musculus
  CDS (1)..(21)
cggatgtccaaccttgcctca

SEQ ID NO: 64 - protein, 7 aa, Mus musculus
RMSNLAS

SEQ ID NO: 65 - DNA, 27 bp, Mus musculus
  CDS (1)..(27)
atgcaacatctagaatatccgtacacg

SEQ ID NO: 66 - protein, 9 aa, Mus musculus
MQHLEYPYT

SEQ ID NO: 67 - DNA, 18 bp, Mus musculus
  CDS (1)..(18)
attgattatacctggaac

SEQ ID NO: 68 - protein, 6 aa, Mus musculus
IDYTWN

SEQ ID NO: 69 - DNA, 48 bp, Mus musculus
  CDS (1)..(48)
tatataacgtacagtggtagcactgactacaacccatctctcaaaagt

SEQ ID NO: 70 - protein, 16 aa, Mus musculus
YITYSGSTDYNPSLKS

SEQ ID NO: 71 - DNA, 27 bp, Mus musculus
  CDS (1)..(27)
ctgggacgtcgctatgctttggactat

SEQ ID NO: 72 - protein, 9 aa, Mus musculus
LGRRYALDY

SEQ ID NO: 73 - DNA, 15 bp, Mus musculus
  CDS (1)..(15)
aactcctggatgaac

SEQ ID NO: 74 - protein, 5 aa, Mus musculus
NSWMN

SEQ ID NO: 75 - DNA, 51 bp, Mus musculus
  CDS (1)..(51)
cggatttatcctggagatggagagactagctacaatggggagttcgtgggc

SEQ ID NO: 76 - protein, 17 aa, Mus musculus
RIYPGDGETSYNGEFVG

SEQ ID NO: 77 - DNA, 18 bp, Mus musculus
  CDS (1)..(18)
tactatgaagggggttac

SEQ ID NO: 78 - protein, 6 aa, Mus musculus
YYEGGY

SEQ ID NO: 79 - DNA, 15 bp, Mus musculus
  CDS (1)..(15)
agctcctggatgaac

SEQ ID NO: 80 - protein, 5 aa, Mus musculus
SSWMN

SEQ ID NO: 81 - DNA, 51 bp, Mus musculus
  CDS (1)..(51)
cggatttatcctggagatggagagactagctacaatggggagttcaagggc

SEQ ID NO: 82 - protein, 17 aa, Mus musculus
RIYPGDGETSYNGEFKG

SEQ ID NO: 83 - DNA, 18 bp, Mus musculus
  CDS (1)..(18)
tactatgaagggggttac

SEQ ID NO: 84 - protein, 6 aa, Mus musculus
YYEGGY

SEQ ID NO: 85 - DNA, 324 bp, Mus musculus
  CDS (1)..(324)
gacatccagatgacacaatcttcatcctccttttctgtgtctctaggagacagagtcaccattacttgcaaggcaagtgaggacatatatattcgcttagcctggtatcagcagaaaccaggaaatgctcctaggctcttaatatctgctgcaaccagtttggaaactgggattccttcaagattcagtggcagtggatctggagaggattacactctcaccattaccagtcttcagactgaagatgttgctacttattactgtcaacaatattggactactccgtggacgttcggtggaggcaccaagctggaaatcaaacgg

SEQ ID NO: 86 - protein, 108 aa, Mus musculus
DIQMTQSSSSFSVSLGDRVTITCKASEDIYIRLAWYQQKPGNAPRLLISAATSLETGIPSRFSGSGSGEDYTLTITSLQTEDVATYYCQQYWTTPWTFGGGTKLEIKR

SEQ ID NO: 87 - DNA, 354 bp, Mus musculus
  CDS (1)..(354)
gatgtgcaacttcaggagtcgggacctggcctggtgaaaccttctcagtctctgtccctcacctgcactgtcactggctactcaatcaccattgattatacctggaactggatccggcagtttccaggaaacaaactggagtggatgggctatataacgtacagtggtagcactgactacaacccatctctcaaaagtcgaagctctatcactcgagacacatccatgaaccagttcttcctgcaattgaattctgtgactactgaggacacagccacatattactgtgcaagactgggacgtcgctatgctttggactattggggtcaaggaacctcagtcaccgtctcctca

SEQ ID NO: 88 - protein, 118 aa, Mus musculus
DVQLQESGPGLVKPSQSLSLTCTVTGYSITIDYTWNWIRQFPGNKLEWMGYITYSGSTDYNPSLKSRSSITRDTSMNQFFLQLNSVTTEDTATYYCARLGRRYALDYWGQGTSVTVSS

SEQ ID NO: 89 - DNA, 339 bp, Mus musculus
  CDS (1)..(339)
gatattgtgatgactcaggctgcaccctctgtacctgtcactcctggagagtcagtatccatctcctgcaggtctagtaagagtctcctgcatagtaatggcaacacttacttgtattggttcctgcagaggccaggccagtctcctcaactcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgtacacgttcggaggggggaccaagctggaaataaaacgg

SEQ ID NO: 90 - protein, 113 aa, Mus musculus
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPYTFGGGTKLEIKR

SEQ ID NO: 91 - DNA, 342 bp, Mus musculus
  CDS (1)..(342)
caggttcagctacagcagtctggacctgagctggtgaagcctggggcctcagtgaagatttcctgcaaggcttctggctacggattcagtaactcctggatgaactgggtgaggcagaggcctggaaagggtcttgagtggattggacggatttatcctggagatggagagactagctacaatggggagttcgtgggcaaggccacactgactgcagacaaatcttccagcacagcctacatgcacctcagcagcctgacatctgaggactctgcggtctacttctgtgcaagctactatgaagggggttactggggccaagggactctggtcactgtctct

SEQ ID NO: 92 - protein, 114 aa, Mus musculus
QVQLQQSGPELVKPGASVKISCKASGYGFSNSWMNWVRQRPGKGLEWIGRIYPGDGETSYNGEFVGKATLTADKSSSTAYMHLSSLTSEDSAVYFCASYYEGGYWGQGTLVTVS

SEQ ID NO: 93 - DNA, 339 bp, Mus musculus
  CDS (1)..(339)
gatattgtgatgactcaggctgcaccctctcttcctgtcactcctggagagtcagtatccatctcctgcaggtctagtaagagtctcctgcatagtaatggcaacacttacttgttttggttcctgcagaggccaggccagtctcctcacctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgtacacgttcggaggggggaccaagctggaaataaaacgg

SEQ ID NO: 94 - protein, 113 aa, Mus musculus
DIVMTQAAPSLPVTPGESVSISCRSSKSLLHSNGNTYLFWFLQRPGQSPHLLIYRMSNLASGVPDRFSGSGSGTAFTLRISRVEAEDVGVYYCMQHLEYPYTFGGGTKLEIKR

SEQ ID NO: 95 - DNA, 345 bp, Mus musculus
  CDS (1)..(345)
caggttcagctgcagcagtctggacctgagctggtgaagcctggggcctcagtgaagatttcctgcaaggcttctggctacggattcagtagctcctggatgaactgggtgaagcagaggcctggaaagggtcttgagtggattggacggatttatcctggagatggagagactagctacaatggggagttcaagggcaaggccacactgactgccgacaaatcctccagcacagcctacatgcaactcagcagcctgacatctgaggactctgcggtctacttctgcgcaagctactatgaagggggttactggggccaagggactctggtcactgtctctgca

SEQ ID NO: 96 - protein, 115 aa, Mus musculus
QVQLQQSGPELVKPGASVKISCKASGYGFSSSWMNWVKQRPGKGLEWIGRIYPGDGETSYNGEFKGKATLTADKSSSTAYMQLSSLTSEDSAVYFCASYYEGGYWGQGTLVTVSA

SEQ ID NO: 97 - protein, 7 aa, Homo sapiens
PWQDGPK

SEQ ID NO: 98 - DNA, 1908 bp, Homo sapiens
  CDS (1)..(1908)
atgccctcctgggccctcttcatggtcacctcctgcctcctcctggcccctcaaaacctggcccaagtcagcagccaagatgtctccttgctggcatcagactcagagcccctgaagtgtttctcccgaacatttgaggacctcacttgcttctgggatgaggaagaggcagcgcccagtgggacataccagctgctgtatgcctacccgcgggagaagccccgtgcttgccccctgagttcccagagcatgccccactttggaacccgatacgtgtgccagtttccagaccaggaggaagtgcgtctcttctttccgctgcacctctgggtgaagaatgtgttcctaaaccagactcggactcagcgagtcctctttgtggacagtgtaggcctgccggctccccccagtatcatcaaggccatgggtgggagccagccaggggaacttcagatcagctgggaggagccagctccagaaatcagtgatttcctgaggtacgaactccgctatggccccagagatcccaagaactccactggtcccacggtcatacagctgattgccacagaaacctgctgccctgctctgcagaggcctcactcagcctctgctctggaccagtctccatgtgctcagcccacaatgccctggcaagatggaccaaagcagacctccccaagtagagaagcttcagctctgacagcagagggtggaagctgcctcatctcaggactccagcctggcaactcctactggctgcagctgcgcagcgaacctgatgggatctccctcggtggctcctggggatcctggtccctccctgtgactgtggacctgcctggagatgcagtggcacttggactgcaatgctttaccttggacctgaagaatgttacctgtcaatggcagcaacaggaccatgctagctcccaaggcttcttctaccacagcagggcacggtgctgccccagagacaggtaccccatctgggagaactgcgaagaggaagagaaaacaaatccaggactacagaccccacagttctctcgctgccacttcaagtcacgaaatgacagcattattcacatccttgtggaggtgaccacagccccgggtactgttcacagctacctgggctcccctttctggatccaccaggctgtgcgcctccccaccccaaacttgcactggagggagatctccagtgggcatctggaattggagtggcagcacccatcgtcctgggcagcccaagagacctgttatcaactccgatacacaggagaaggccatcaggactggaaggtgctggagccgcctctcggggcccgaggagggaccctggagctgcgcccgcgatctcgctaccgtttacagctgcgcgccaggctcaacggccccacctaccaaggtccctggagctcgtggtcggacccaactagggtggagaccgccaccgagaccgcctggatctccttggtgaccgctctgcatctagtgctgggcctcagcgccgtcctgggcctgctgctgctgaggtggcagtttcctgcacactacaggagactgaggcatgccctgtggccctcacttccagacctgcaccgggtcctaggccagtaccttagggacactgcagccctgagcccgcccaaggccacagtctcagatacctgtgaagaagtggaacccagcctccttgaaatcctccccaagtcctcagagaggactcctttgcccctgtgttcctcccaggcccagatggactaccgaagattgcagccttcttgcctggggaccatgcccctgtctgtgtgcccacccatggctgagtcagggtcctgctgtaccacccacattgccaaccattcctacctaccactaagctattggcagcagccttga

SEQ ID NO: 99 - protein, 635 aa, Homo sapiens
MPSWALFMVTSCLLLAPQNLAQVSSQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP